(19) United States
(12) Patent Application Publication
(10) Pub. No.: US 2026/0043027 A1
(43) Pub. Date: Feb. 12, 2026

(54) LINKER, COMPOUND INCLUDING LINKER, METHOD OF PREPARING COMPOUND, USE OF LINKER, AND METHOD OF DELIVERING OLIGONUCLEOTIDES THROUGH LINKER

(71) Applicant: SynerK Biotech Limited, Hong Kong (HK)

(72) Inventors: Dong YU, Westboro, MA (US); Tao LAN, Brookline, MA (US); Weiwen JIANG, Winchester, MA (US)

(21) Appl. No.: 19/277,676

(22) Filed: Jul. 23, 2025

Related U.S. Application Data
(60) Provisional application No. 63/678,247, filed on Aug. 1, 2024.

(51) Int. Cl. C12N 15/113; A61K 31/7125; C07H 1/00; C07H 21/02

(52) U.S. Cl. CPC C12N 15/113; A61K 31/7125; C07H 1/00; C07H 21/02; C12N 2310/11; C12N 2310/14; C12N 2310/315; C12N 2310/3519

(57) ABSTRACT

The present disclosure relates to the technical field of biological delivery, and discloses a linker with a specific structure, a compound including the linker, a method of preparing the compound, use of the linker, and a method of delivering oligonucleotides through the linker. The linker of the present disclosure may be linked to the oligonucleotides, and may be hydrolyzed to release the oligonucleotides linked thereto, and the oligonucleotides bind to the same or different positions of the same target mRNA, or the oligonucleotides bind to two or more target mRNAs.

### RELATED APPLICATION

This application claims the benefit of U.S. Provisional Application No. 63/678,247, filed on Aug. 1, 2024. The entire teachings of the above application are incorporated herein by reference.

### SEQUENCE LISTING

The sequence listing submitted via EFS, in compliance with 37 CFR § 1.52 (e) (5), is incorporated herein by reference. The sequence listing XML file submitted via EFS contains the file “42693008US1SubSL.xml”, created on Nov. 3, 2025, which is 45,379 bytes in size.

### TECHNICAL FIELD

The present disclosure relates to the field of biological delivery, in particular to a linker, a compound including the linker, a method of preparing the compound, use of the linker, and a method of delivering oligonucleotides through the linker. The present invention is directed to compounds, compositions, and methods useful for modulating gene expression.

### BACKGROUND

Oligonucleotide delivery technology has made significant contributions to human health. The oligonucleotide delivery technology not only enables targeting of oligonucleotides to pathogenic genes that are inaccessible to traditional small molecules/antibodies and achieves long-term gene silencing with a single dose while reducing side effects (for example, Zilebesiran can sustain effects for up to 6 months), but also enables the oligonucleotides to act directly on the root cause of genetic diseases, thereby eradicating the diseases from the source.

Although linkers for oligonucleotide delivery exist in the art, there remains an urgent need for novel hydrolysable linkers capable of simultaneously delivering two or more oligonucleotides.

### SUMMARY

Therefore, the technical problem to be solved by the present disclosure is how to provide a novel hydrolysable linker.

Another technical problem to be solved by the present disclosure is improving inhibition of gene activity and/or stability defects of a compound including the linker, thereby providing a compound including a specific linker.

To this end, in a first aspect, the present disclosure provides a linker, comprising:
a structure selected from the group consisting of:

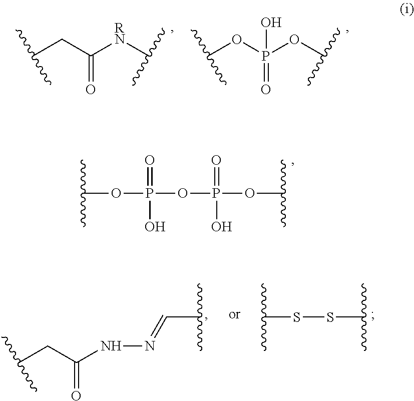

(ii) a combination of

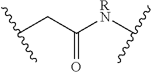

and one or more of

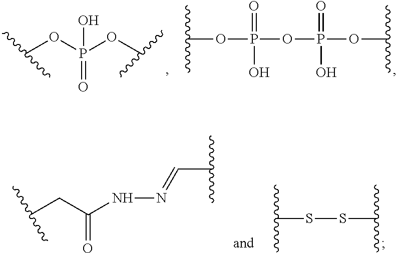

or

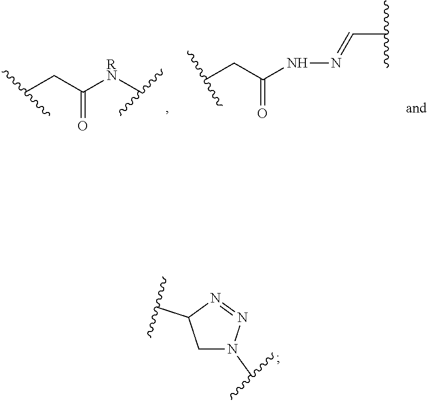

(iii) a combination of
wherein R is selected from —H, —CO(CH₂)_{d}CH₃, —(CH₂)_{d}CH₃, —CH(CH₃)₂, —(CH₂)_{d}OH, —(CH₂)_{d}COOH, —CO(CH₂)_{d}CH₃, —(CH₂)_{d}CONH₂, —(CH₂)_{d}NHCOH, —(CH₂)_{d}NH₂, —(CH₂)_{d}NHCO(CH₂)ₑCH₃, or —(CH₂)_{d}CONH(CH₂)ₑCH₃, wherein d and e are independently selected from integers ranging from 0 to 20; the linker is hydrolysable. The linker may be linked to oligonucleotides through positions shown by wavy lines, and may be hydrolyzed to release the oligonucleotides linked thereto, and the oligonucleotides bind to the same or different positions of the same target mRNA, or the oligonucleotides bind to two or more target mRNAs.

In an optional embodiment, the linker comprising a structure selected from the group consisting of formulas A, B, C, E, and F, as shown below:

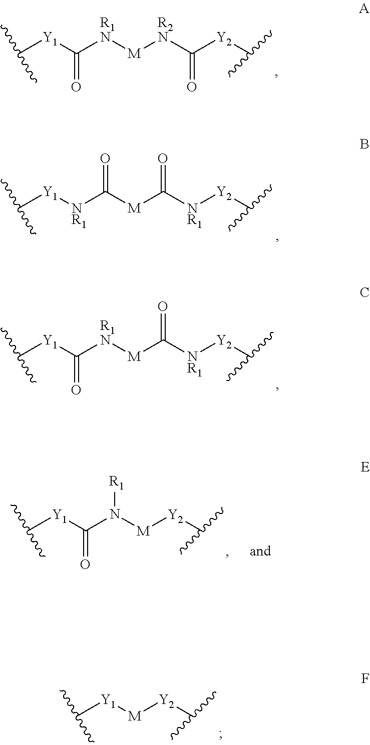

wherein M is selected from the group consisting of: a C1-C30 alkylidene group, a substituted C1-C30 alkylidene group, a C2-C30 alkenylidene group, a substituted C2-C30 alkenylidene group, a C2-C30 alkynylene group, a substituted C2-C30 alkynylene group, a nitrogen-containing functional group, a phosphorus-containing functional group, an oxygen-containing functional group, a sulfur-containing functional group, a silicon-containing functional group, and a gold-containing functional group; wherein M is optionally attached to an oligonucleotide and/or a ligand;
Y₁ and Y₂ are independently selected from —[CH₂]_{m′}(CHQ′)[CH₂]_{n′}—, —(CH₂)_{m′}NQ′(CH₂)_{n′}—, —(CH₂)_{m′}OP(═O)(X′)O(CH₂)_{n′}—, —(CH₂)_{m′}(OCH₂CH₂)_{t′}O(CH₂)_{n′}—, —(CH₂)^{m′}(CH═CH)_{t′}(CH₂)_{n′}—, —(CH₂)_{m′}(C₆H₄)(CH₂)_{n′}—, —(CH₂)_{m′}CO(CH₂)_{n′}—, —(CH₂)_{m′}(C₆H₁₀)(CH₂)_{n′}—, —(CH₂)_{m′}(C₆H₁₀O₆)(CH₂)_{n′}—, —(CH₂)_{m′}—O—SiO—O—(CH₂)_{n′}—, —[CH₂]_{m′}(CHQ′)[CH₂]_{n′}O—, —(CH₂)_{m′}NQ′ (CH₂)_{n′}O—, —(CH₂)_{m′}OP(═O)(X′)O(CH₂)_{n′}O—, —(CH₂)_{n′}OP(═O)OHO—, —(CH₂)_{n′}OP(═S)OHO—, —(CH₂)_{m′}S—S(CH₂)_{n′}O—, —(CH₂)_{m′}(CH═CH)_{t″}(CH₂)_{n′}O—, —(CH₂)_{m′}(C₆H₄)(CH₂)_{n′}O—, —(CH₂)_{m′}CO(CH₂)_{n′}O—, —(CH₂)_{m′}(C₆H₁₀)(CH₂)_{n′}O—, —(CH₂)_{m′}(C₆H₁₀O₆)(CH₂)_{n′}O—, —(CH₂)_{m′}—O—SiO—O—(CH₂)_{n′}O—, —[CH₂]_{m′}(CHQ′)[CH₂]_{n′}S—, —(CH₂)_{m′}NQ′ (CH₂)_{n′}S—, —(CH₂)_{m′}OP(═O)(X′)O(CH₂)_{n′}S—, —(CH₂)_{m′}S—S(CH₂)_{n′}S—, —(CH₂)_{m′}(CH═CH)_{t′}(CH₂)_{n′}S—, —(CH₂)_{m′}(C₆H₄)(CH₂)_{n′}S—, —(CH₂)_{m′}CO(CH₂)_{n′}S—, —(CH₂)_{m′}(C₆H₁₀)(CH₂)_{n′}S—, —(CH₂)_{m′}(C₆H₁₀O₆)(CH₂)_{n′}S—, —(CH₂)_{m′}—O—SiO—O—(CH₂)_{n′}S—,

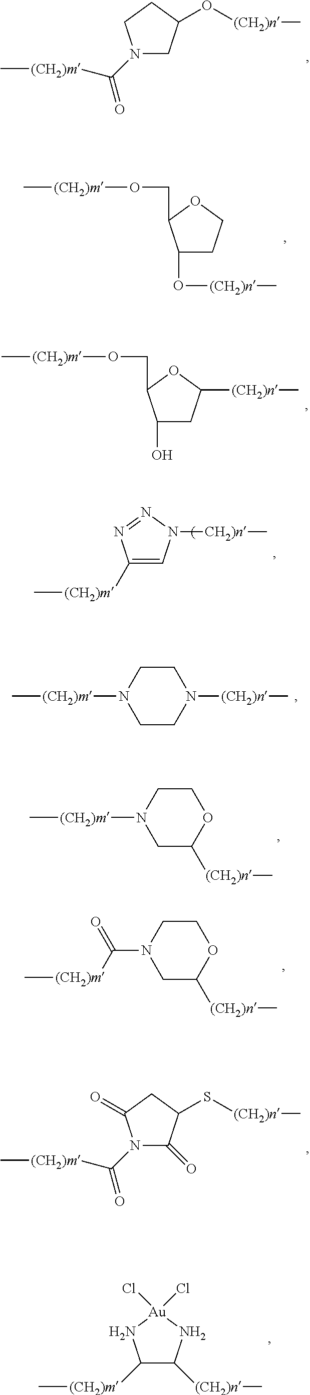

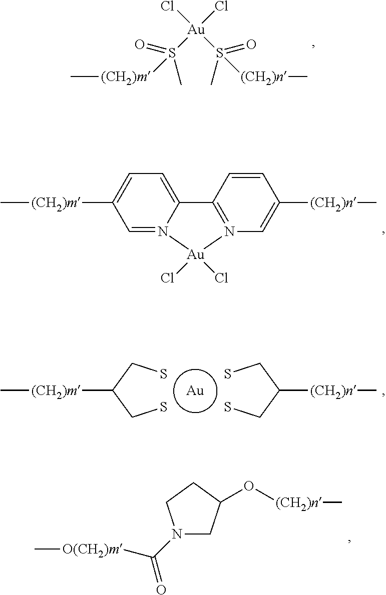

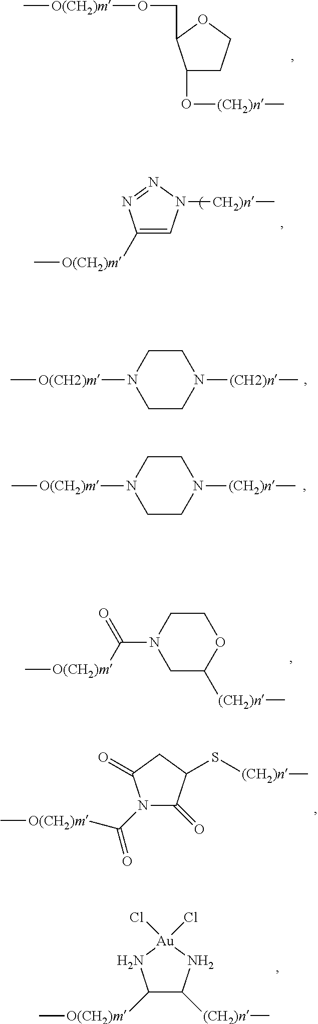

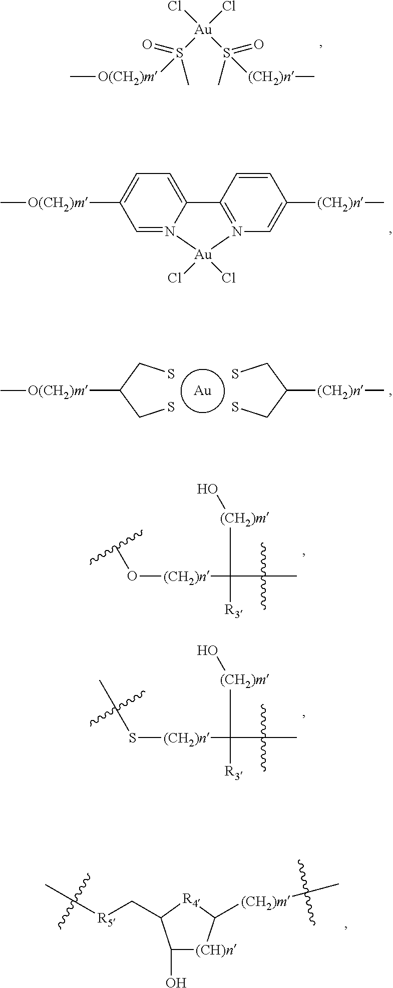

—(CH₂)_{m′}O(CH₂)_{n′}O—, —(CH₂)_{m′}OP(═S)(Y′)O(CH₂)_{n′}—, —(CH₂)_{m′}NHCO(CH₂)_{n′}—, —(CH₂)_{m′}O—(CH₂)_{n′}—, —(CH₂)_{l′}(OCH₂CH₂)_{n′}O(CH₂)_{m′}CONH—,

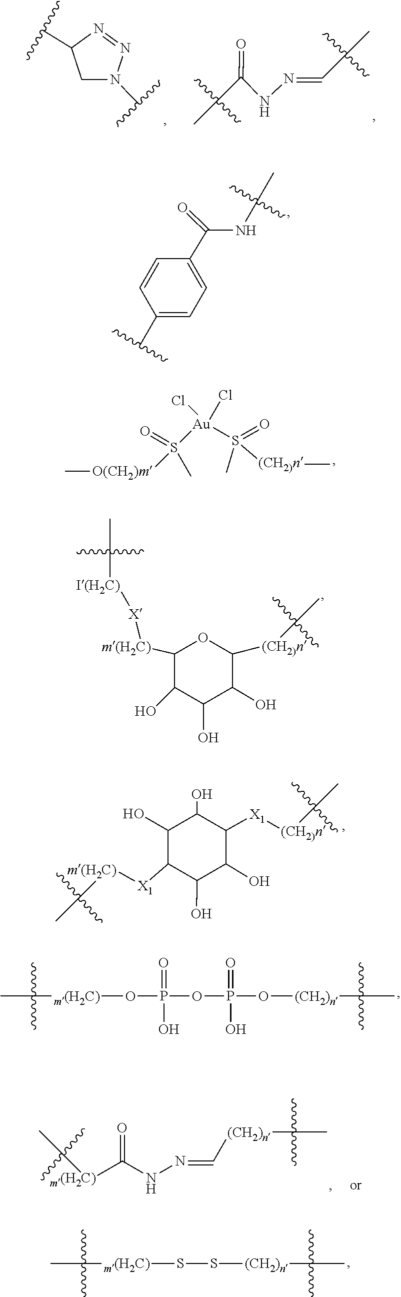

wherein l′, n′, m′, and t′ are independently selected from the integers ranging from 0 through 20; wherein Y₁ and Y₂ are the same or different;
R_{3′} is selected from H, —Cl, —Br, —F, —OH, —(CH₂)_{x′}CH₃, or —O(CH₂)_{x′}CH₃; wherein x′ is an integer ranging from 0 to 20;
R_{4′} and R_{5′} are independently selected from —O—, —S—, —CH₂—, —NH—, —HCH₃—, or —NCH₃—;
X′ and Y′ are each independently selected from OH, SH, O, O⁻, S, S⁻, —NH₂, —CH₃, or —OCH₃; Q′ is selected from —H, —F, —Cl, —Br, —I, —(CH₂)_{a′}CH₃, —CH(CH₃)₂, —(CH₂)_{a′}OH, —(CH₂)_{a′}COOH, —CO(CH₂)_{a′}CH₃, —(CH₂)_{a′}NHCO(CH₂)_{b′}O—, —(CH₂)_{a′}CONH(CH₂)_{b′}O—, —(CH₂)_{a′}CONH—NH═CH(CH₂)_{b′}—, —(CH₂)_{a′}CONH—NH═CH(CH₂)_{b′}O—, —(CH₂)_{a′}CO(CH₂)_{b′}O—, —(CH₂)_{a′}CONH₂, —(CH₂)_{a′}NHCOH, —(CH₂)_{a′}NH₂, —(CH₂)_{a′}NHCO(CH₂)_{b′}CH₃, —(CH₂)_{a′}CONH(CH₂)_{b′}CH₃, —O(CH₂)_{a′}O—, —(CH₂)_{a′}(OCH₂CH₂)_{b′}O(CH₂)_{c′}—, —OPO₃H—, —O—PSO₂H—, —OP(═O)(OH)₂, —OP(═S)(OH)₂, —OP(═O)(CH₂)_{a′}CH₃OH, —OP(═O)(NH₂)OH, —OP(═O)[O(CH₂)_{a′}CH₃]OH, —OP(═O)[NH(CH₂)_{a′}CH₃]OH, —OP(═S)(CH₂)_{a′}CH₃OH, —OP(═S)[O(CH₂)_{a′}CH₃]OH, —OP(═S)(NH₂)OH, —OP(═S)ₙH(CH₂)_{a′}CH₃OH, —OP(═S)SHOH, —OS(═O)₂OH, —OS(═O)OH, —(CH₂)_{a′}OP(═O)OHO—, —(CH₂)_{a′}OP(═S)OHO—, —OP(═O)(CH₂)_{a′}CH₃O—, —OP(═O)(OCH₃)O—, —OP(═O)(NH₂)O—, —(CH₂)_{a′}O—, —(OCH₂CH₂)_{a′}—O—, —(CH═CH—CH₂)_{a′}O—, —(C₆H₄—CH₂—)_{a′}O—, —(C₆H₁₀)_{a′}O—, —(C₆H₁₀—CH₂—)_{a′}O—, wherein a′, b′, and c′ are independently selected from integers ranging from 0 through 20;
R₁ and R₂ are independently selected from —H, —CO(CH₂)_{d}CH₃, —(CH₂)_{d}CH₃, —CH(CH₃)₂, —(CH₂)_{d}OH, —(CH₂)_{d}COOH, —CO(CH₂)_{d}CH₃, —(CH₂)_{d}CONH₂, —(CH₂)_{d}NHCOH, —(CH₂)_{d}NH₂, —(CH₂)_{d}NHCO(CH₂)ₑCH₃, or —(CH₂)_{d}CONH(CH₂)ₑCH₃, wherein d and e are independently selected from integers ranging from 0 to 20.

In an optional embodiment, M has a symmetrical structure.

In an optional embodiment, R₁ and R₂ are independently selected from —H or —(CH₂)_{d}CH₃.

In an optional embodiment, M is independently selected from —[CH₂]ₘ(CHQ)[CH₂]ₙ—, —(CH₂)ₘNQ(CH₂)ₙ, —(CH₂)ₘOP(═O)(X)O(CH₂)ₙ, —(CH₂)ₘ(OCH₂CH₂)ₜO(CH₂)ₙ, —(CH₂)ₘ(CH═CH)ₜ(CH₂)ₙ, —(CH₂)ₘ(C₆H₄)(CH₂)ₙ, —(CH₂)ₘCO(CH₂)ₙ, —(CH₂)ₘ(C₆H₁₀)(CH₂)ₙ, —(CH₂)ₘ(C₆H₁₀O₆)(CH₂)ₙ, —(CH₂)ₘ—O—SiO—O—(CH₂)ₙ, —[CH₂]ₘ(CHQ)[CH₂]ₙO, —(CH₂)ₘNQ(CH₂)ₙO, —(CH₂)ₘOP(═O)(X)O(CH₂)ₙO, —(CH₂)ₙOP(═O)OHO, —(CH₂)ₙOP(═S)OHO, —(CH₂)ₘS—S(CH₂)ₙO, —(CH₂)ₘ(CH═CH)ₜ(CH₂)ₙO, —(CH₂)ₘ(C₆H₄)(CH₂)ₙO, —(CH₂)ₘCO(CH₂)ₙO, —(CH₂)ₘ(C₆H₁₀)(CH₂)ₙO, —(CH₂)ₘ(C₆H₁₀O₆)(CH₂)ₙO, —(CH₂)ₘ—O—SiO—O—(CH₂)ₙO, —[CH₂]ₘ(CHQ)[CH₂]ₙS, —(CH₂)ₘNQ(CH₂)ₙS, —(CH₂)ₘOP(═O)(X)O(CH₂)ₙS, —(CH₂)ₘS—S(CH₂)ₙS, —(CH₂)ₘ(CH═CH)ₜ(CH₂)ₙS, —(CH₂)ₘ(C₆H₄)(CH₂)ₙS, —(CH₂)ₘCO(CH₂)ₙS, —(CH₂)ₘ(C₆H₁₀)(CH₂)ₙS, —(CH₂)ₘ(C₆H₁₀O₆)(CH₂)ₙS, —(CH₂)ₘ—O—SiO—O—(CH₂)ₙS,

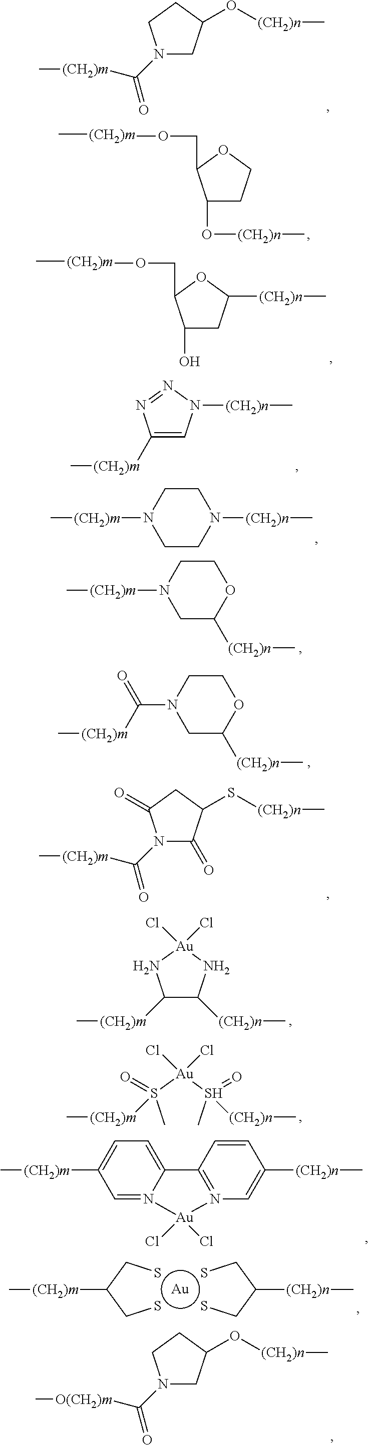

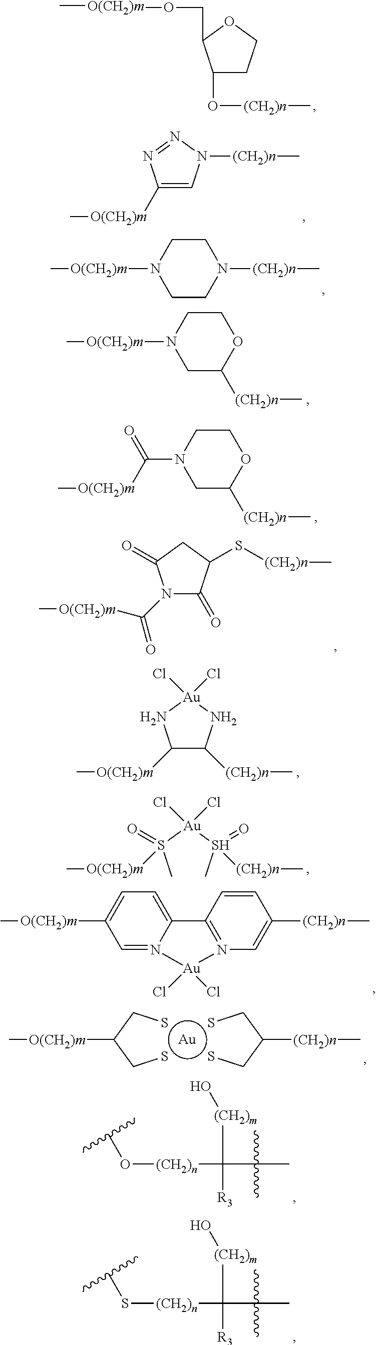

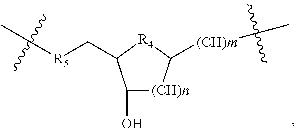

—(CH₂)ₘO(CH₂)ₙO, —(CH₂)ₘOP(═S)(Y)O(CH₂)ₙ, —(CH₂)ₘNHCO(CH₂)ₙ, —(CH₂)ₘO—(CH₂)ₙ, —(CH₂)(OCH₂CH₂)ₙO(CH₂)ₘCONH,

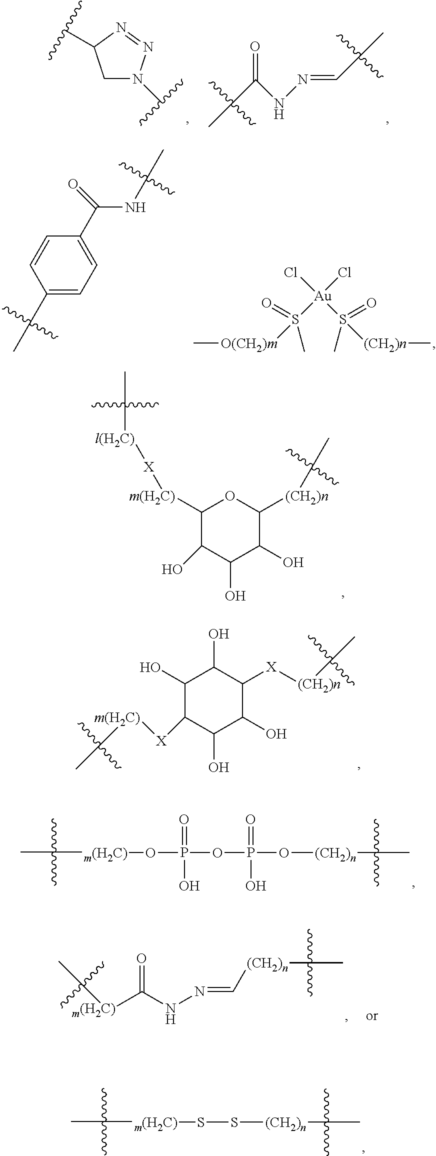

wherein l, n, m, and t are independently selected from integers ranging from 0 through 20.

In an optional embodiment, M is selected from —(CH₂)ₘNQ(CH₂)ₙ—, —[CH₂]ₘ(CHQ)[CH₂]ₙ—, (CH₂)ₘ(CH═CH)ₜ(CH₂)ₙ—, —(CH₂)ₘOP(═O)(X)O(CH₂)ₙ—, —(CH₂)ₘOP(═S)(Y)O(CH₂)ₙ—, —(CH₂)ₘ(OCH₂CH₂)ₜO(CH₂)ₙ—, —(CH₂)ₘ—O—SiO—O—(CH₂)ₙ—,

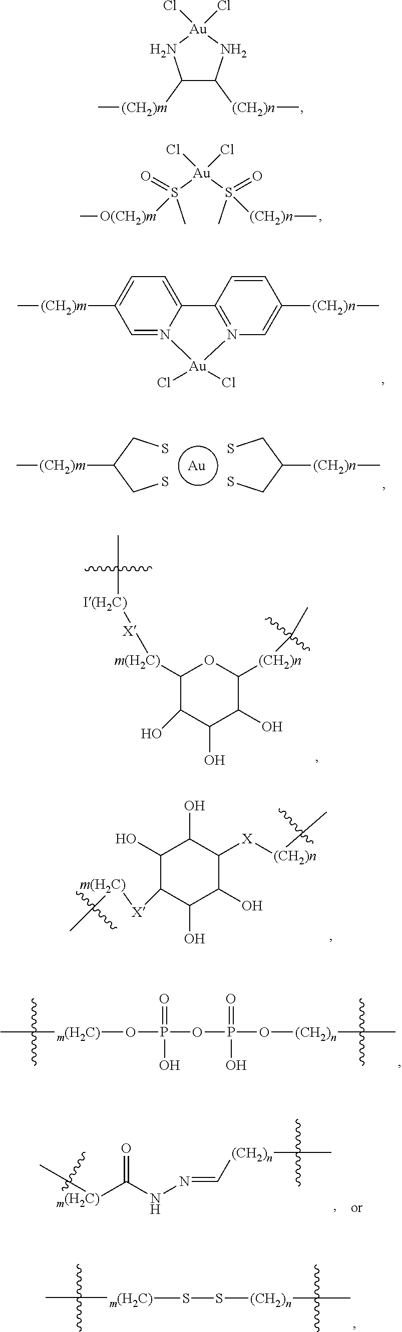

wherein Q is —(CH₂)ₐ(OCH₂CH₂)_{b}O(CH₂)_{c}—, —(CH₂)_{d}CH₃, —(CH₂)ₐCO(CH₂)_{b}O—, or —(CH₂)_{d}CONH(CH₂)_{b}O—, wherein l, n, m, t, a, b, and c independently are selected from integers ranging from 0 to 20, and X and Y are each independently selected from OH, SH, O, O⁻, S, S⁻, —NH₂, —CH₃, or —OCH₃.

In an optional embodiment, M is selected from:
(a) M is a C₁-C₃₀ alkylidene group,
(b) when M is —[CH₂]ₘ(CHQ)[CH₂]ₙ—, Q is —(CH₂)ₐ(OCH₂CH₂)_{b}O(CH₂)_{c}—, a, b, and c are each 0, and neither m nor n is 0;
(c) when M is —(CH₂)ₘ(CH═CH)ₜ(CH₂)ₙ—, neither m, n nor t is 0;
(d) when M is —(CH₂)ₘNQ(CH₂)ₙ—, neither m nor n is 0, Q is —(CH₂)_{d}CH₃, —(CH₂)ₐCO(CH₂)_{b}O—, or —(CH₂)_{d}CONH(CH₂)_{b}O—;
(e) when M is —(CH₂)ₘOP(═O)(X)O(CH₂)ₙ—, neither m nor n is 0, X is OH, SH, O⁻ or S⁻;
(f) when M is —(CH₂)ₘOP(═S) (Y)O(CH₂)ₙ—, neither m nor n is 0, and Y is selected from OH, SH, O⁻ or S⁻;
(g) when M is —(CH₂)ₘ(OCH₂CH₂)ₜO(CH₂)ₙ—, neither m nor n nor t is 0;
(h) when M is

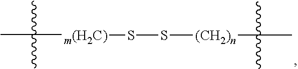

neither m nor n is 0;
(i) when M is —(CH₂)ₘ—O—SiO—O—(CH₂)ₙ—, neither m nor n is 0;
(j) when M is

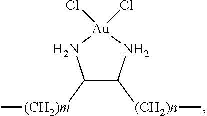

neither m nor n is 0;
(k) when M is

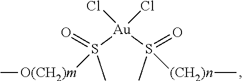

neither m nor n is 0;
(l) when M is

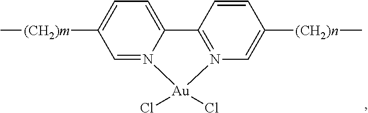

neither m nor n is 0;
(m) when M is

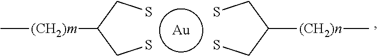

neither m nor n is 0;
(n) when M is

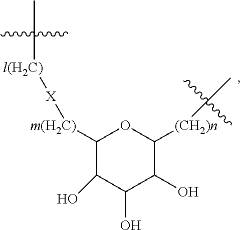

X is O or S, neither 1, m, nor n is 0;
(o) when M is

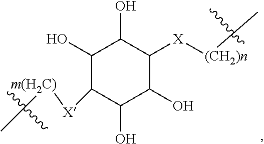

X is O or S, neither m nor n is 0;
(p) when M is

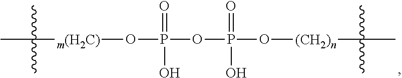

neither m nor n is 0; or
(q) when M is

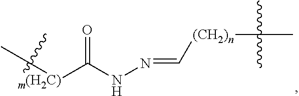

neither m nor n is 0.

In an optional embodiment,
(a) when M is —(CH₂)ₘNQ(CH₂)ₙ—,
   (i) if Q is —(CH₂)ₐCO(CH₂)_{b}O—, then a is 0 or a non-zero integer, and b is a non-zero integer; and
   (ii) if Q is —(CH₂)_{d}CONH(CH₂)_{b}O—, then neither a nor b is 0; or
(b) when M is

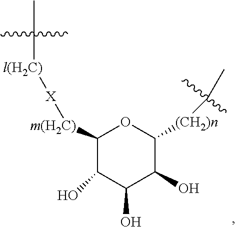

X is O or S, and neither l, m, nor n is 0; or
(c) when M is

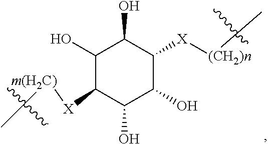

X is O or S, and neither m nor n is 0.

In an optional embodiment, m=n.

In an optional embodiment, Y₁ and Y₂ are independently selected from: —(CH₂)_{m′}(OCH₂CH₂)_{t′}O(CH₂)_{n′}—, —(CH₂)_{m′}O(CH₂)_{n′}O—, —[CH₂]_{m′}(CHQ′)[CH₂]_{n′}O—, —[CH₂]_{m′}(CHQ′)[CH₂]_{n′}S—,

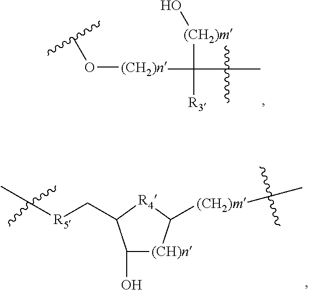

(CH₂)_{m′}NHCO(CH₂)_{n′}—, —(CH₂)_{m′}O—(CH₂)_{n′}—, —(CH₂)_{l′}(OCH₂CH₂)_{n′}O(CH₂)_{m′}CONH—,

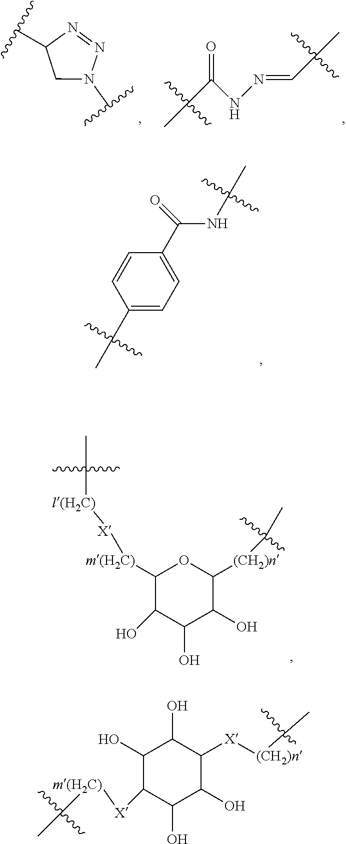

or combinations thereof.

In an optional embodiment, one end of Y₁ or Y₂ is an oxygen (O) or sulfur (S) atom bonded to other structural fragment beyond Y₁ or Y₂, the O or S atom is bonded through a wavy line shown in any one of formulas A, B, C, E, or F.

In an optional embodiment, Y₁ and Y₂ are the same.

In an optional embodiment,
(a) when Y₁ or Y₂ is —(CH₂)_{m′}(OCH₂CH₂)_{n′}O(CH₂)_{n′}—, the values of m′, n′, and t′ satisfy any one of the following conditions:
   (i) m′=0, t′=0, and n′≠0;
   (ii) m′=0, n′=0, and t′≠0;
   (iii) m′≠0, t′≠0, and n′=0;
   (iv) n′=0, t′=0, and m′≠0;
   (v) m′=0, t′≠0, and n′≠0;
(b) when Y₁ or Y₂ is —(CH₂)_{m′}O(CH₂)_{n′}O—, neither m′ nor n′ is 0;
(c) when Y₁ or Y₂ is —[CH₂]_{m′}(CHQ′)[CH₂]_{n′}O—, neither m′ nor n′ is 0, and Q′ is H or —(CH₂)_{d}CH₃;
(d) when Y₁ or Y₂ is —[CH₂]_{m′}(CHQ′)[CH₂]_{n′}S—, Q′ is —H, neither m′ nor n′ is 0;
(e) when Y₁ or Y₂ is

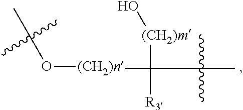

R₃ is —(CH₂)_{x′}CH₃;
(f) when Y₁ or Y₂ is

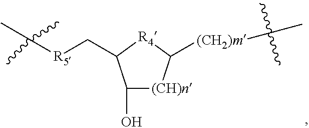

R₄ and R₅ are each —O—, neither m′ nor n′ is 0;
(g) when Y₁ or Y₂ is a combination of groups selected from —(CH₂)_{m′}NHCO(CH₂)_{n′}—,

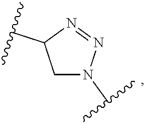 [SMILES: *C1CN(*)N=N1]

—(CH₂)_{m′}O—(CH₂)_{n′}—,

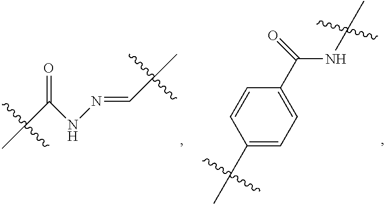 [SMILES: */C=N/NC(*)=O ; *NC(=O)c1ccc(*)cc1]

and —(CH₂)_{l′}(OCH₂CH₂)_{n′}O(CH₂)_{m′}CONH—, the groups are connected in sequence, wherein neither l′, m′, nor n′ is 0;
(h) when Y₁ or Y₂ is

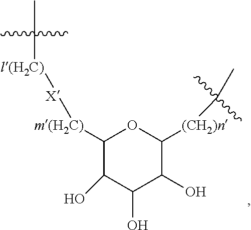

X′ is either O or S, and neither m′ nor n′ is 0;
(i) when Y₁ or Y₂ is

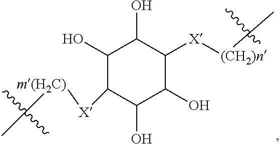

X′ is either O or S, and neither m′ nor n′ is 0;
j) when Y₁ or Y₂ is

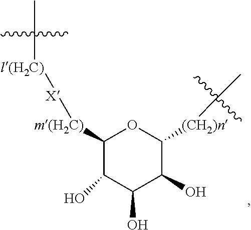

X′ is either O or S, and neither m′ nor n′ is 0;
(k) when Y₁ or Y₂ is

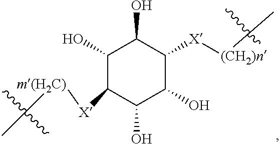

X′ is either O or S, and neither m′ nor n′ is 0.

In an optional embodiment, (a) M is a C₁-C₃₀ alkylene group; and Y₁ and Y₂ are selected from —(CH)_{m′}(OCH₂CH₂)_{t′}O(CH₂)—, —(CH₂)_{m′}O(CH₂)_{n′}O—, —[CH₂]_{m′}(CHQ′)[CH₂]_{n′}O—, —[CH₂]_{m′}(CHQ′)[CH₂]_{n′}S—,

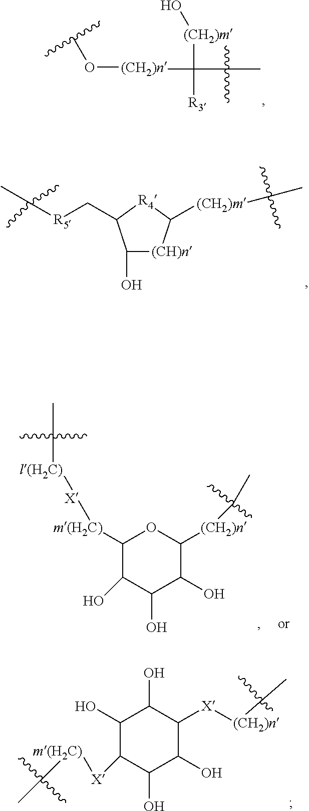

(b) when M is —[CH₂]ₘ(CHQ)[CH₂]ₙ—, Q is —(CH₂)_{a′}(OCH₂CH₂)_{b}O(CH₂)_{c}—, wherein a, b, and c are each 0, and neither m nor n is 0; and Y₁ and/or Y₂ is —(CH₂)ₘ(OCH₂CH₂)_{t′}O(CH₂)_{n′}—, wherein m′ and t′ are each 0 and n′ is a non-zero integer;
(c) when M is —(CH₂)ₘ(CH═CH)ₜ(CH₂)ₙ—, neither m, n, nor t is 0; and Y₁ and/or Y₂ is —(CH₂)_{m′}(OCH₂CH₂)_{t′}O(CH₂)_{n′}—, wherein n′ and t′ are each 0, and m′ is a non-zero integer;
(d) when M is —(CH₂)ₘNQ(CH₂)ₙ—, neither m nor n is 0, Q is selected from —(CH₂)_{d}CH₃, —(CH₂)ₐCO(CH₂)_{b}O—, or —(CH₂)ₐCONH(CH₂)_{b}O—; and Y₁ and/or Y₂ is —(CH₂)_{m′}(OCH₂CH₂)_{t′}O(CH₂)_{n′}—, wherein n′ and t′ are each 0, and m′ is a non-zero integer;
(e) when M is —(CH₂)ₘOP(═O)(X)O(CH₂)ₙ—, neither m nor n is 0, and X is selected from OH, SH, O⁻, or S⁻; and Y₁ and/or Y₂ is —(CH₂)ₘ(OCH₂CH₂)_{t′}O(CH₂)_{n′}—, wherein n′ and t′ are each 0, and m′ is a non-zero integer;
(f) when M is —(CH₂)ₘOP(═S) (Y)O(CH₂)ₙ—, neither m nor n is 0, and Y is selected from OH, SH, O⁻, or S⁻; and Y₁ and/or Y₂ is —(CH₂)_{m′}(OCH₂CH₂)_{t′}O(CH₂)_{n′}—, wherein n′ and t′ are each 0, and m′ is a non-zero integer;
(g) when M is —(CH₂)ₘ(OCH₂CH₂)ₜO(CH₂)ₙ—, neither m, n, nor t is 0; and Y₁ and/or Y₂ is —(CH₂)_{m′}(OCH₂CH₂)_{t′}O(CH₂)_{n′}—, wherein n′ and t′ are each 0, and m′ is a non-zero integer;
(h) when M is

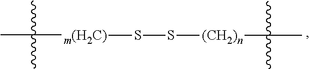

neither m nor n is 0; and Y₁ and/or Y₂ is —(CH₂)_{m′}(OCH₂CH₂)_{t′}O(CH₂)_{n′}—, wherein n′ and t′ are each 0, and m′ is a non-zero integer;
(i) when M is —(CH₂)ₘ—O—SiO—O—(CH₂)ₙ—, neither m nor n is 0; and Y₁ and/or Y₂ is —(CH₂)_{m′}(OCH₂CH₂)_{t′}O(CH₂)_{n′}—, wherein n′ and t′ are each 0, and m′ is a non-zero integer;
(j) when M is

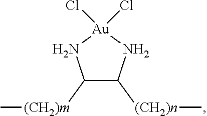

neither m nor n is 0; and Y₁ and/or Y₂ is —(CH₂)_{m′}(OCH₂CH₂)_{t′}O(CH₂)_{n′}—, wherein n′ and t′ are each 0, and m′ is a non-zero integer;
(k) when M is

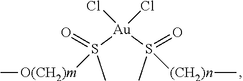

neither m nor n is 0; and Y₁ and/or Y₂ is —(CH₂)_{m′}(OCH₂CH₂)_{t′}O(CH₂)_{n′}—, wherein n′ and t′ are each 0, and m′ is a non-zero integer;
(l) when M is

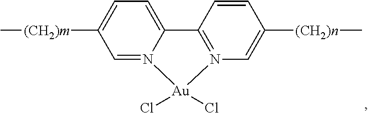

neither m nor n is 0; and Y₁ and/or Y₂ is —(CH₂)_{m′}(OCH₂CH₂)_{t′}O(CH₂)_{n′}—, wherein n′ and t′ are each 0, and m′ is a non-zero integer;
(m) when M is

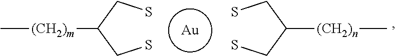

neither m nor n is 0; and Y₁ and/or Y₂ is —(CH₂)_{m′}(OCH₂CH₂)_{t′}O(CH₂)_{n′}—, wherein n′ and t′ are each 0, and m′ is a non-zero integer;
(n) when M is

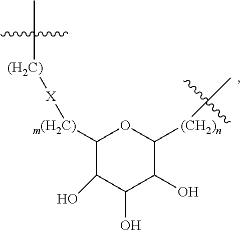

X is O or S, neither m nor n is 0; and Y₁ and/or Y₂ is —(CH₂)_{m′}(OCH₂CH₂)_{t′}O(CH₂)_{n′}—, wherein neither n′ nor t′ is 0, and m′ is 0;
(o) when M is

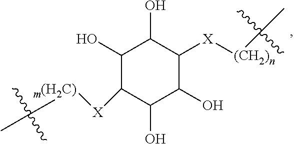

neither m nor n is 0; and Y₁ and/or Y₂ is —(CH₂)_{m′}(OCH₂CH₂)_{t′}O(CH₂)_{n′}—, wherein n′ and t′ are each 0, and m′ is a non-zero integer;
(p) when M is —(CH₂)ₙOP(═O)OHO—, n is 0; and Y₁ and/or Y₂ is —(CH₂)_{m′}(OCH₂CH₂)_{t′}O(CH₂)_{n′}—, wherein n′ is 0 and neither m′ nor t is 0;
(q) when M is

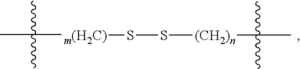

neither m nor n is 0; and Y₁ and/or Y₂ is —(CH₂)_{m′}(OCH₂CH₂)_{t′}O(CH₂)_{n′}—, wherein t′ is a non-zero integer, and n′ and m′ are each 0;
(r) when M is

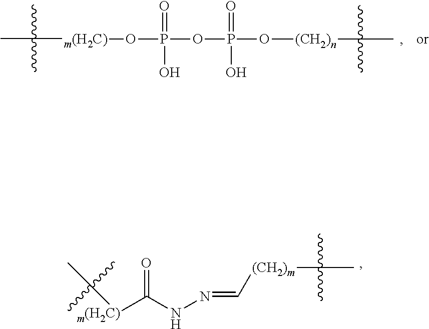

neither m nor n is 0; and Y₁ and/or Y₂ is —(CH₂)_{m′}(OCH₂CH₂)_{t′}O(CH₂)_{n′}—, wherein n′ and t′ are each 0, and m′ is a non-zero integer; or Y₁ and/or Y₂ is —(CH₂)_{m′}(OCH₂CH₂)_{t′}O(CH₂)_{n′}—, wherein m′ is 0, n′ and t′ are each a non-zero integer;
(s) M is a C₁-C₃₀ alkylene group; and Y₁ and/or Y₂ is a combination of groups selected from: —(CH₂)_{m′}NHCO(CH₂)_{n′}—,

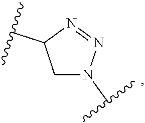 [SMILES: *C1CN(*)N=N1]

—(CH₂)_{m′}O—(CH₂)_{n′}—,

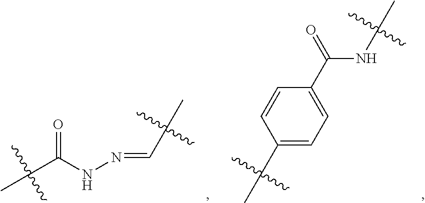 [SMILES: */C=N/NC(*)=O ; *NC(=O)c1ccc(*)cc1]

and —(CH₂)_{l′}(OCH₂CH₂)_{n′}O(CH₂)_{m′}CONH—, or is

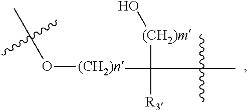

wherein the groups in the combination are connected in sequence, and neither l′, m′ nor n′ is 0.

In an optional embodiment, any of l′, n′, m′, t′, x′, a′, b′, c′, l, n, m, t, x, a, b, c, d, or e is independently selected from an integer falling within any one of the following ranges: 0-15, 0-10, 0-8, 0-7, 0-6, 0-5, 0-4, 0-3, 0-2, 1-15, 1-10, 1-8, 1-7, 1-6, 1-5, 1-3, 1-2.

For the technical solutions involving chemical structures described in the present application, a person skilled in the art can reasonably select the variables described in the present application to form technical solutions that fall within the scope of the present disclosure. The present disclosure excludes technical solutions where the selection of variables violates bonding principles.

In an optional embodiment, the linker comprises a structure shown below:

TABLE 1

| No. | Structure |
|---|---|
| A1 | 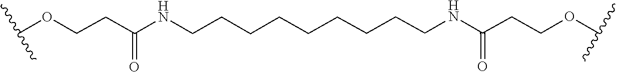 [SMILES: *OCCC(=O)NCCCCCCCCCNC(=O)CCO*] |
| A2 | 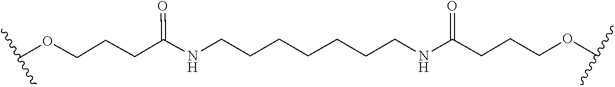 [SMILES: *OCCCC(=O)NCCCCCCCNC(=O)CCCO*] |
| A3 | 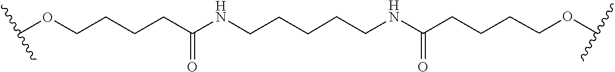 [SMILES: *OCCCCC(=O)NCCCCCNC(=O)CCCCO*] |
| A4 | 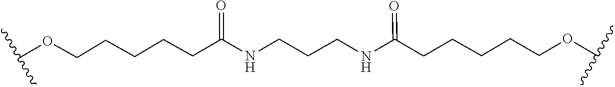 [SMILES: *OCCCCCC(=O)NCCCNC(=O)CCCCCO*] |
| A5 | 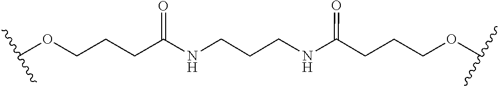 [SMILES: *OCCCC(=O)NCCCNC(=O)CCCO*] |
| A6 | 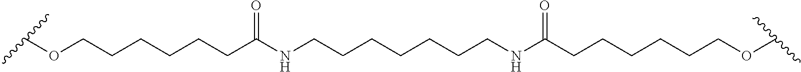 [SMILES: *OCCCCCCC(=O)NCCCCCCCNC(=O)CCCCCCO*] |
| A7 | 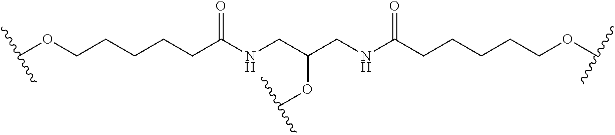 [SMILES: *OCCCCCC(=O)NCC(CNC(=O)CCCCCO*)O*] |
| A8 | 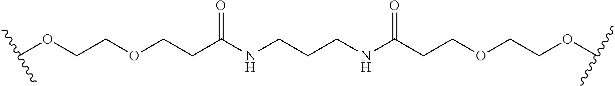 [SMILES: *OCCOCCC(=O)NCCCNC(=O)CCOCCO*] |
| A9 | 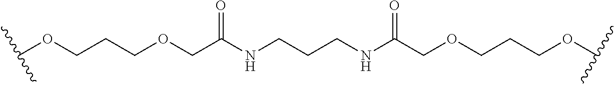 [SMILES: *OCCCOCC(=O)NCCCNC(=O)COCCCO*] |
| A10 | 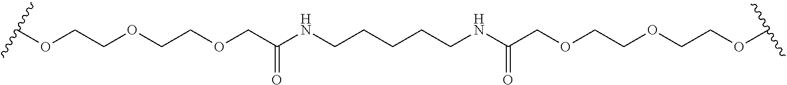 [SMILES: *OCCOCCOCC(=O)NCCCCCNC(=O)COCCOCCO*] |
| A11 | 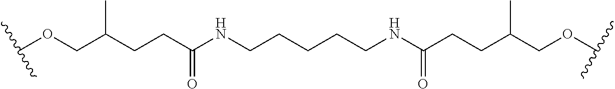 [SMILES: *OCC(C)CCC(=O)NCCCCCNC(=O)CCC(C)CO*] |
| A12 | 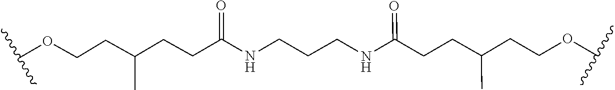 [SMILES: *OCCC(C)CCC(=O)NCCCNC(=O)CCC(C)CCO*] |
| A13 | 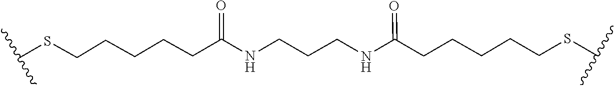 [SMILES: *SCCCCCC(=O)NCCCNC(=O)CCCCCS*] |
| A14 | 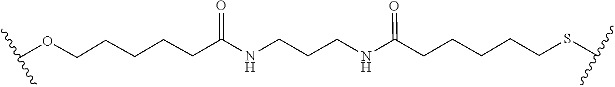 [SMILES: *OCCCCCC(=O)NCCCNC(=O)CCCCCS*] |
| A15 | 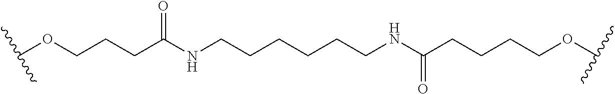 [SMILES: *OCCCCC(=O)NCCCCCCNC(=O)CCCO*] |
| A16 | 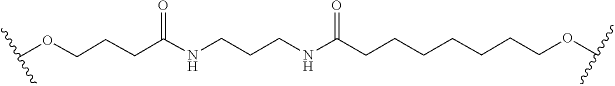 [SMILES: *OCCCCCCCC(=O)NCCCNC(=O)CCCO*] |
| A17 | 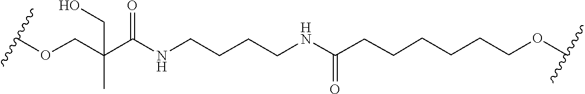 [SMILES: *OCCCCCCC(=O)NCCCCNC(=O)C(C)(CO)CO*] |
| A18 | 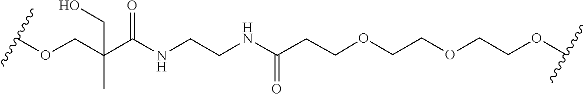 [SMILES: *OCCOCCOCCC(=O)NCCNC(=O)C(C)(CO)CO*] |
| A19 | 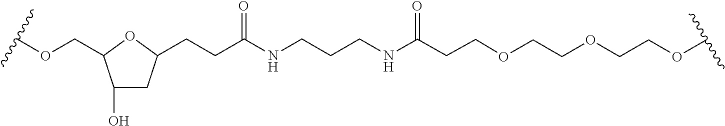 [SMILES: *OCCOCCOCCC(=O)NCCCNC(=O)CCC1CC(O)C(CO*)O1] |
| A20 | 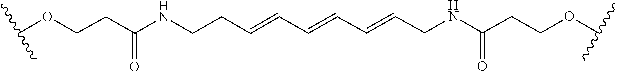 [SMILES: *OCCC(=O)NC/C=C/C=C/C=C/CCNC(=O)CCO*] |
| A21 | 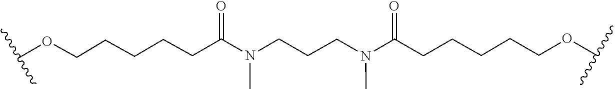 [SMILES: *OCCCCCC(=O)N(C)CCCN(C)C(=O)CCCCCO*] |
| A22 | 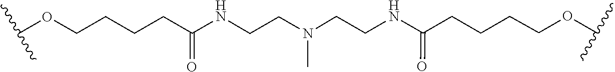 [SMILES: *OCCCCC(=O)NCCN(C)CCNC(=O)CCCCO*] |
| A23 | 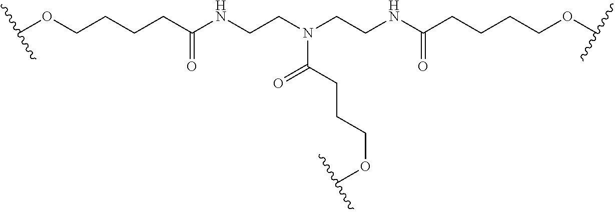 [SMILES: *OCCCCC(=O)NCCN(CCNC(=O)CCCCO*)C(=O)CCCO*] |
| A24 | 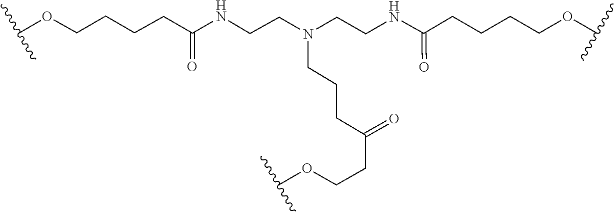 [SMILES: *OCCCCC(=O)NCCN(CCCC(=O)CCO*)CCNC(=O)CCCCO*] |
| A25 | 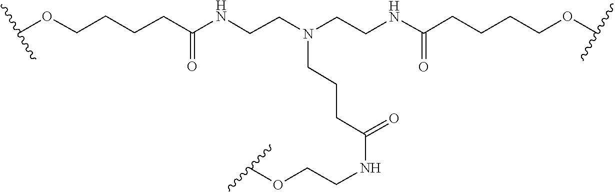 [SMILES: *OCCCCC(=O)NCCN(CCCC(=O)NCCO*)CCNC(=O)CCCCO*] |
| A26 | 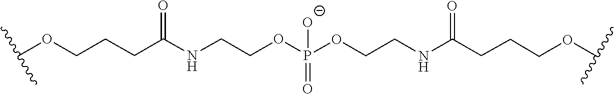 [SMILES: *OCCCC(=O)NCCOP(=O)([O-])OCCNC(=O)CCCO*] |
| A27 | 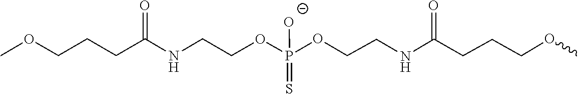 |
| A28 | 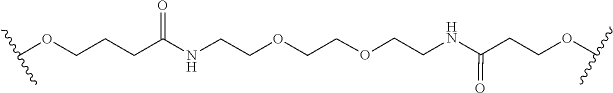 [SMILES: *OCCCC(=O)NCCOCCOCCNC(=O)CCO*] |
| A29 | 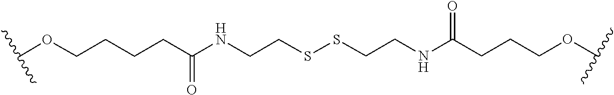 [SMILES: *OCCCCC(=O)NCCSSCCNC(=O)CCCO*] |
| A30 | 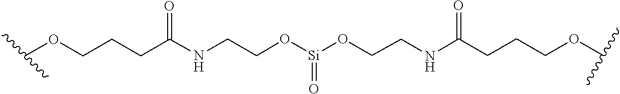 [SMILES: *OCCCC(=O)NCCO[Si](=O)OCCNC(=O)CCCO*] |
| A31 | 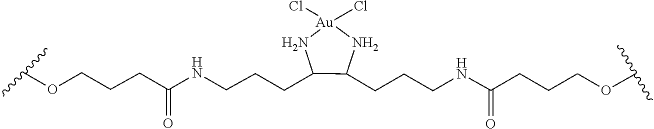 |
| A32 | 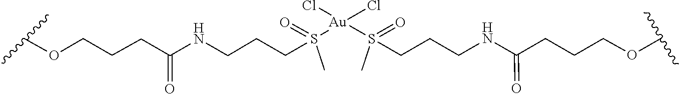 |
| A33 | 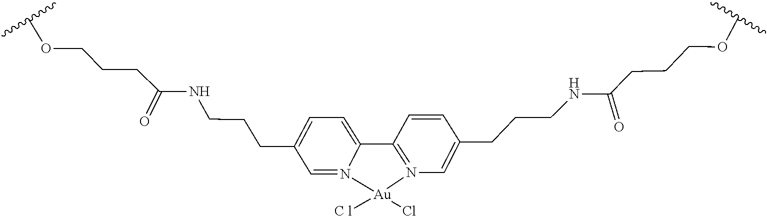 |
| A34 | 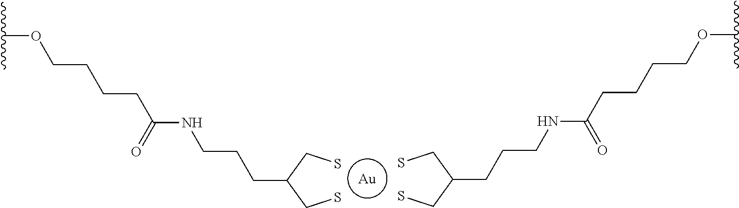 |
| A35 | 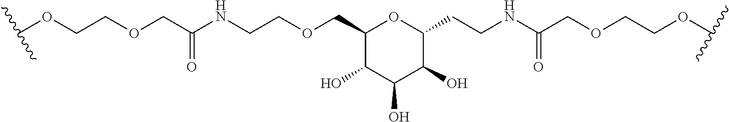 [SMILES: *OCCOCC(=O)NCCOC[C@H]1O[C@H](CCNC(=O)COCCO*)[C@@H](O)[C@@H](O)[C@@H]1O] |
| A36 | 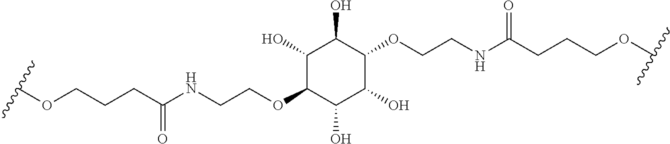 [SMILES: *OCCCC(=O)NCCO[C@H]1[C@H](O)[C@@H](O)[C@H](OCCNC(=O)CCCO*)[C@@H](O)[C@H]1O] |
| A37 | 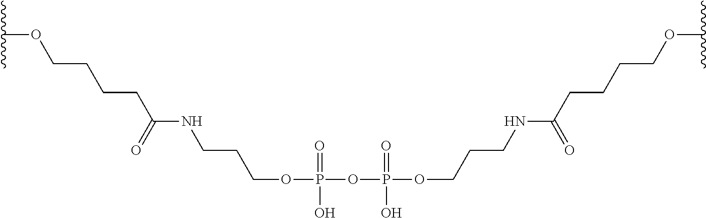 [SMILES: *OCCCCC(=O)NCCCOP(=O)(O)OP(=O)(O)OCCCNC(=O)CCCCO*] |
| A38 | 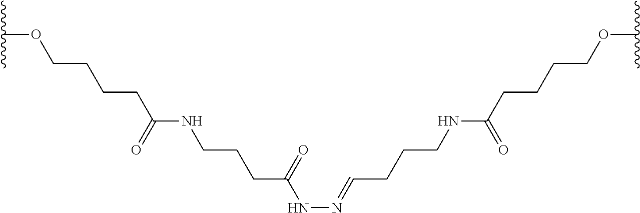 [SMILES: *OCCCCC(=O)NCCC/C=N/NC(=O)CCCNC(=O)CCCCO*] |

TABLE 2

| No. | Structure |
|---|---|
| B1 | 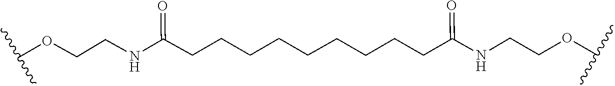 [SMILES: *OCCNC(=O)CCCCCCCCCC(=O)NCCO*] |
| B2 | 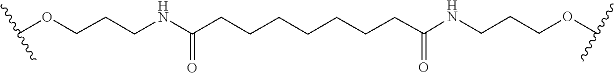 [SMILES: *OCCCNC(=O)CCCCCCCC(=O)NCCCO*] |
| B3 | 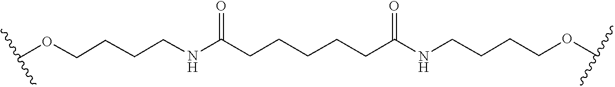 [SMILES: *OCCCCNC(=O)CCCCCC(=O)NCCCCO*] |
| B4 | 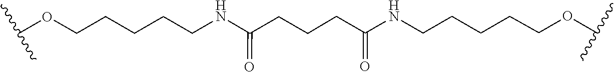 [SMILES: *OCCCCCNC(=O)CCCC(=O)NCCCCCO*] |
| B5 | 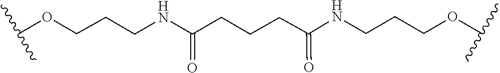 [SMILES: *OCCCNC(=O)CCCC(=O)NCCCO*] |
| B6 | 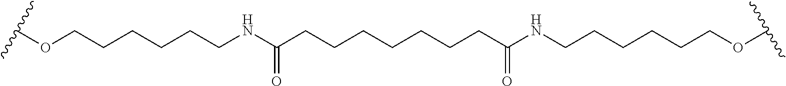 [SMILES: *OCCCCCCNC(=O)CCCCCCCC(=O)NCCCCCCO*] |
| B7 | 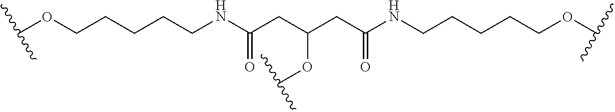 [SMILES: *OCCCCCNC(=O)CC(CC(=O)NCCCCCO*)O*] |
| B8 | 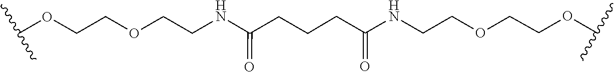 [SMILES: *OCCOCCNC(=O)CCCC(=O)NCCOCCO*] |
| B9 | 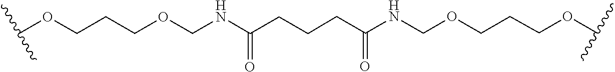 [SMILES: *OCCCOCNC(=O)CCCC(=O)NCOCCCO*] |
| B10 | 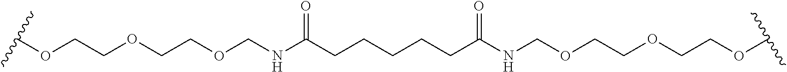 [SMILES: *OCCOCCOCNC(=O)CCCCCC(=O)NCOCCOCCO*] |
| B11 | 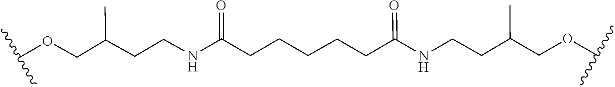 [SMILES: *OCC(C)CCNC(=O)CCCCCC(=O)NCCC(C)CO*] |
| B12 | 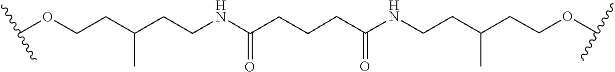 [SMILES: *OCCC(C)CCNC(=O)CCCC(=O)NCCC(C)CCO*] |
| B13 | 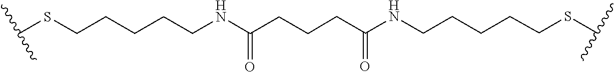 [SMILES: *SCCCCCNC(=O)CCCC(=O)NCCCCCS*] |
| B14 | 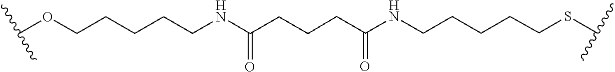 [SMILES: *OCCCCCNC(=O)CCCC(=O)NCCCCCS*] |
| B15 | 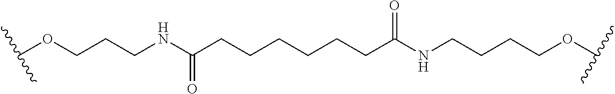 [SMILES: *OCCCCNC(=O)CCCCCCC(=O)NCCCO*] |
| B16 | 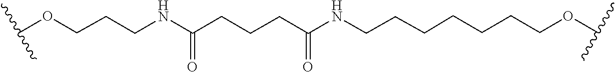 [SMILES: *OCCCCCCCNC(=O)CCCC(=O)NCCCO*] |
| B17 | 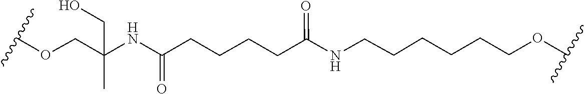 [SMILES: *OCCCCCCNC(=O)CCCCC(=O)NC(C)(CO)CO*] |
| B18 | 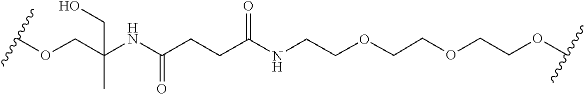 [SMILES: *OCCOCCOCCNC(=O)CCC(=O)NC(C)(CO)CO*] |
| B19 | 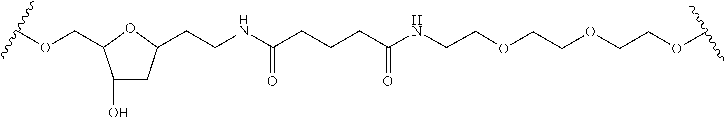 [SMILES: *OCCOCCOCCNC(=O)CCCC(=O)NCCC1CC(O)C(CO*)O1] |
| B20 | 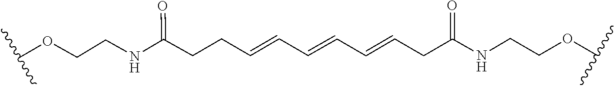 [SMILES: *OCCNC(=O)C/C=C/C=C/C=C/CCC(=O)NCCO*] |
| B21 | 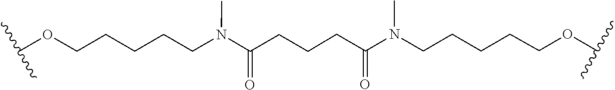 [SMILES: *OCCCCCN(C)C(=O)CCCC(=O)N(C)CCCCCO*] |
| B22 | 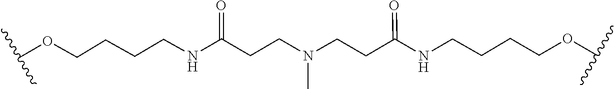 [SMILES: *OCCCCNC(=O)CCN(C)CCC(=O)NCCCCO*] |
| B23 | 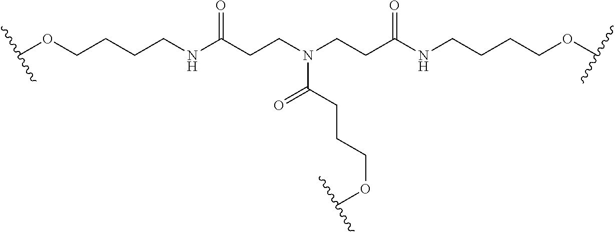 [SMILES: *OCCCCNC(=O)CCN(CCC(=O)NCCCCO*)C(=O)CCCO*] |
| B24 | 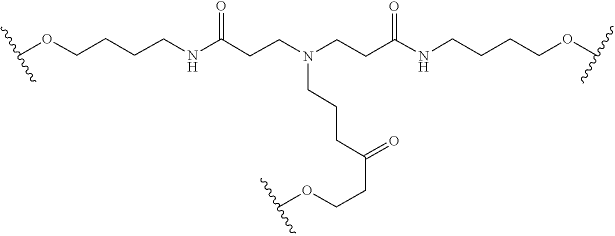 [SMILES: *OCCCCNC(=O)CCN(CCCC(=O)CCO*)CCC(=O)NCCCCO*] |
| B25 | 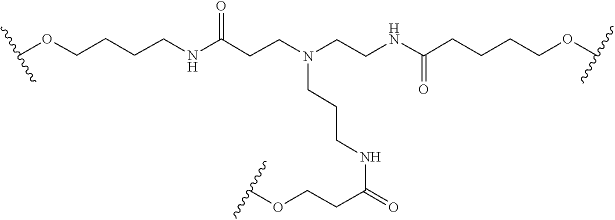 [SMILES: *OCCCCNC(=O)CCN(CCCNC(=O)CCO*)CCNC(=O)CCCCO*] |
| B26 | 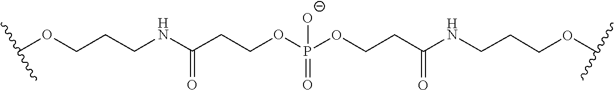 [SMILES: *OCCCNC(=O)CCOP(=O)([O-])OCCC(=O)NCCCO*] |
| B27 | 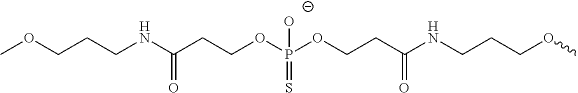 |
| B28 | 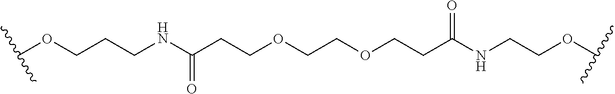 [SMILES: *OCCCNC(=O)CCOCCOCCC(=O)NCCO*] |
| B29 | 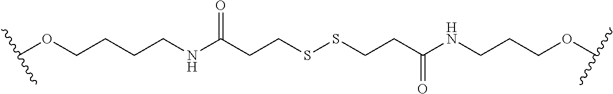 [SMILES: *OCCCCNC(=O)CCSSCCC(=O)NCCCO*] |
| B30 | 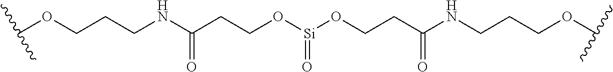 |
| B31 | 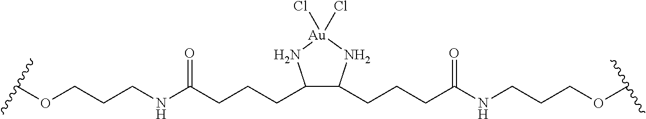 |
| B32 | 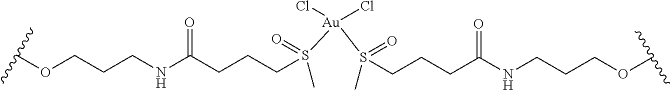 |
| B33 | 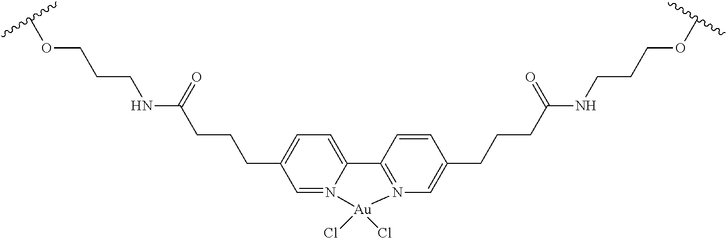 |
| B34 | 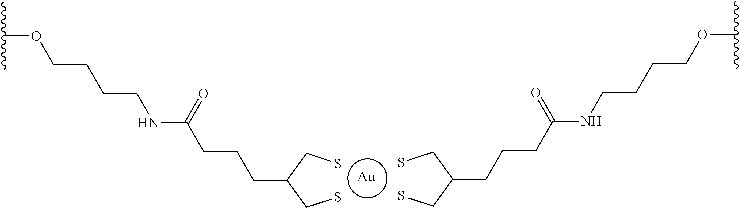 |
| B35 | 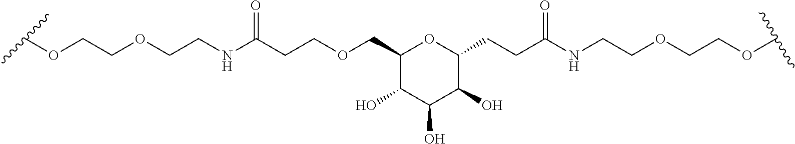 [SMILES: *OCCOCCNC(=O)CCOC[C@H]1O[C@H](CCC(=O)NCCOCCO*)[C@@H](O)[C@@H](O)[C@@H]1O] |
| B36 | 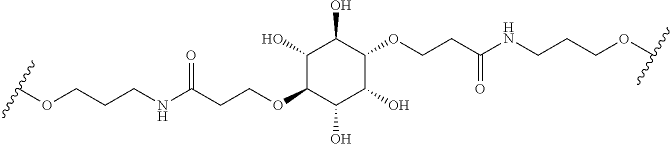 [SMILES: *OCCCNC(=O)CCO[C@H]1[C@H](O)[C@@H](O)[C@H](OCCC(=O)NCCCO*)[C@@H](O)[C@H]1O] |
| B37 | 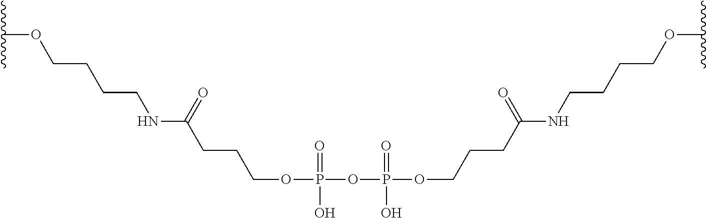 [SMILES: *OCCCCNC(=O)CCCOP(=O)(O)OP(=O)(O)OCCCC(=O)NCCCCO*] |
| B38 | 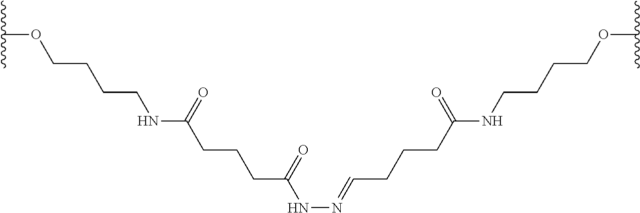 [SMILES: *OCCCCNC(=O)CCC/C=N/NC(=O)CCCC(=O)NCCCCO*] |

TABLE 3

| No. | Structure |
|---|---|
| C1 | 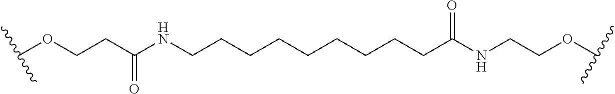 [SMILES: *OCCNC(=O)CCCCCCCCCNC(=O)CCO*] |
| C2 | 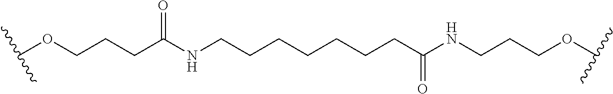 [SMILES: *OCCCNC(=O)CCCCCCCNC(=O)CCCO*] |
| C3 | 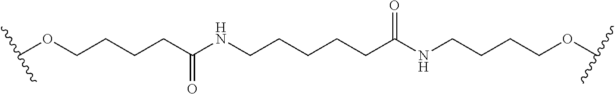 [SMILES: *OCCCCNC(=O)CCCCCNC(=O)CCCCO*] |
| C4 | 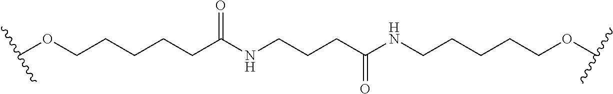 [SMILES: *OCCCCCNC(=O)CCCNC(=O)CCCCCO*] |
| C5 | 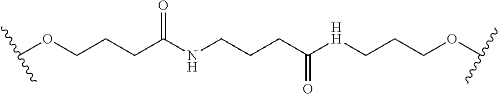 |
| C6 | 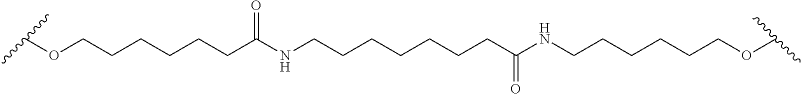 [SMILES: *OCCCCCCNC(=O)CCCCCCCNC(=O)CCCCCCO*] |
| C7 | 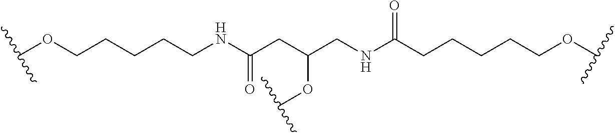 [SMILES: *OCCCCCNC(=O)CC(CNC(=O)CCCCCO*)O*] |
| C8 | 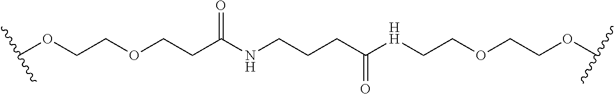 |
| C9 | 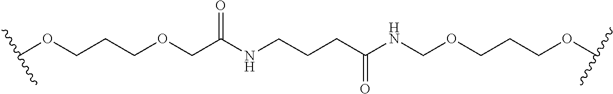 [SMILES: *OCCCOCNC(=O)CCCNC(=O)COCCCO*] |
| C10 | 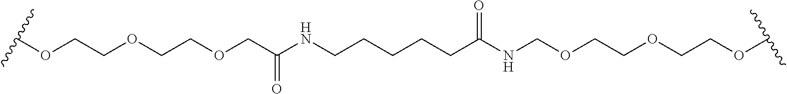 [SMILES: *OCCOCCOCNC(=O)CCCCCNC(=O)COCCOCCO*] |
| C11 | 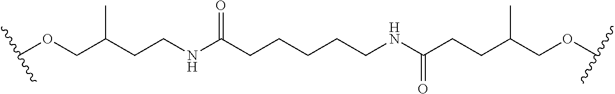 [SMILES: *OCC(C)CCNC(=O)CCCCCNC(=O)CCC(C)CO*] |
| C12 | 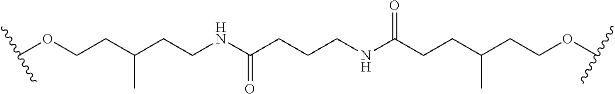 [SMILES: *OCCC(C)CCNC(=O)CCCNC(=O)CCC(C)CCO*] |
| C13 | 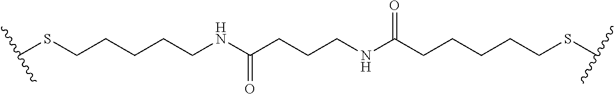 [SMILES: *SCCCCCNC(=O)CCCNC(=O)CCCCCS*] |
| C14 | 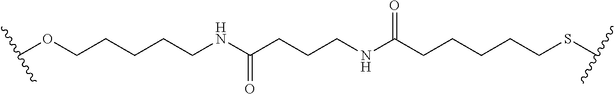 [SMILES: *OCCCCCNC(=O)CCCNC(=O)CCCCCS*] |
| C15 | 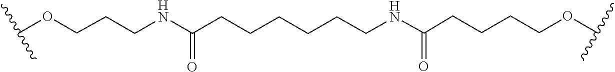 [SMILES: *OCCCCC(=O)NCCCCCCC(=O)NCCCO*] |
| C16 | 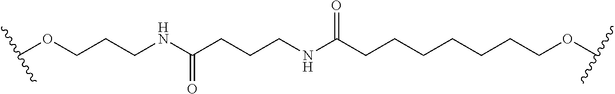 [SMILES: *OCCCCCCCC(=O)NCCCC(=O)NCCCO*] |
| C17 | 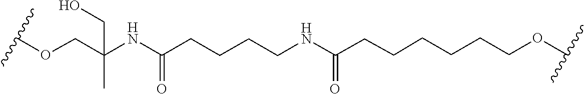 [SMILES: *OCCCCCCC(=O)NCCCCC(=O)NC(C)(CO)CO*] |
| C18 | 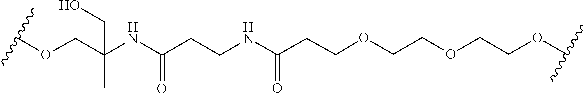 [SMILES: *OCCOCCOCCC(=O)NCCC(=O)NC(C)(CO)CO*] |
| C19 | 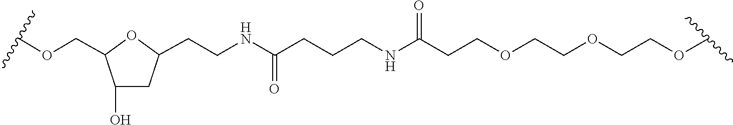 [SMILES: *OCCOCCOCCC(=O)NCCCC(=O)NCCC1CC(O)C(CO*)O1] |
| C20 | 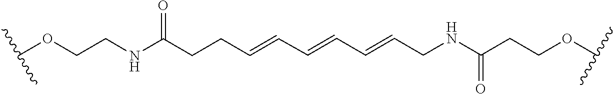 [SMILES: *OCCNC(=O)CC/C=C/C=C/C=C/CNC(=O)CCO*] |
| C21 | 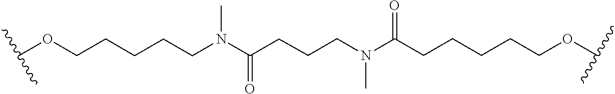 [SMILES: *OCCCCCC(=O)N(C)CCCC(=O)N(C)CCCCCO*] |
| C22 | 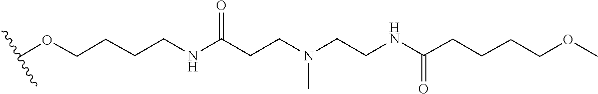 [SMILES: *OCCCCNC(=O)CCN(C)CCNC(=O)CCCCOC] |
| C23 | 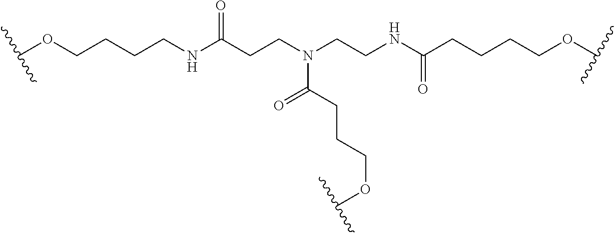 [SMILES: *OCCCCNC(=O)CCN(CCNC(=O)CCCCO*)C(=O)CCCO*] |
| C24 | 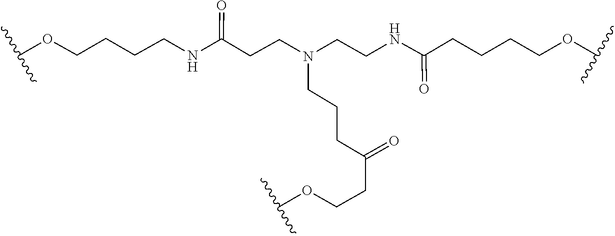 [SMILES: *OCCCCNC(=O)CCN(CCCC(=O)CCO*)CCNC(=O)CCCCO*] |
| C25 | 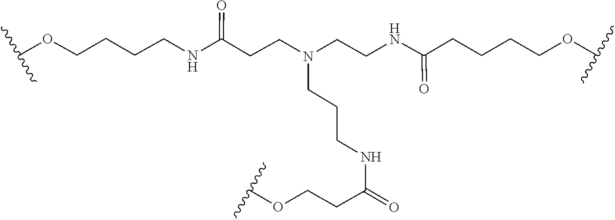 [SMILES: *OCCCCNC(=O)CCN(CCCNC(=O)CCO*)CCNC(=O)CCCCO*] |
| C26 | 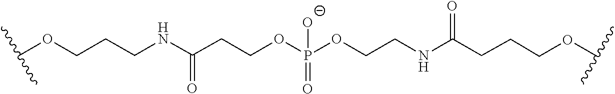 [SMILES: *OCCCNC(=O)CCOP(=O)([O-])OCCNC(=O)CCCO*] |
| C27 | 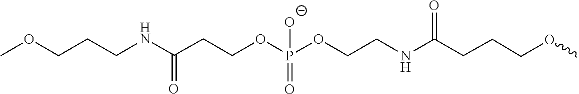 |
| C28 | 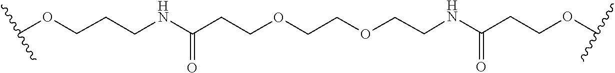 [SMILES: *OCCCNC(=O)CCOCCOCCNC(=O)CCO*] |
| C29 | 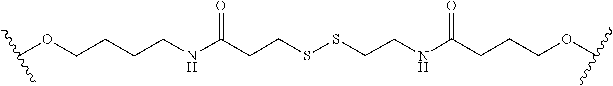 [SMILES: *OCCCCNC(=O)CCSSCCNC(=O)CCCO*] |
| C30 | 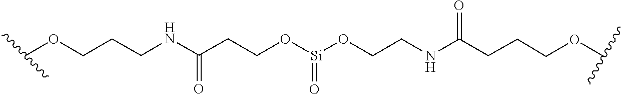 [SMILES: *OCCCNC(=O)CCO[Si](=O)OCCNC(=O)CCCO*] |
| C31 | 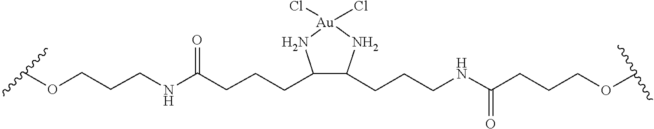 |
| C32 | 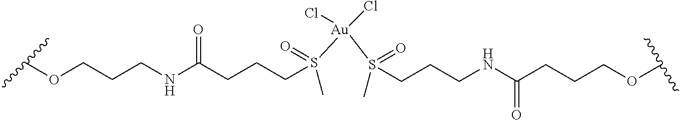 |
| C33 | 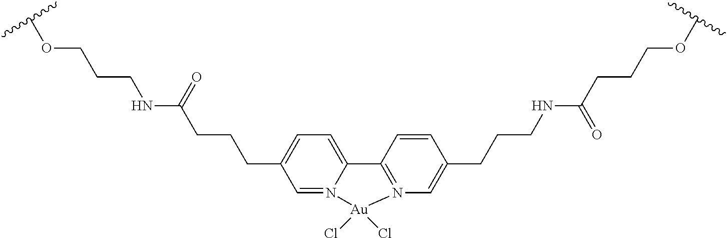 |
| C34 | 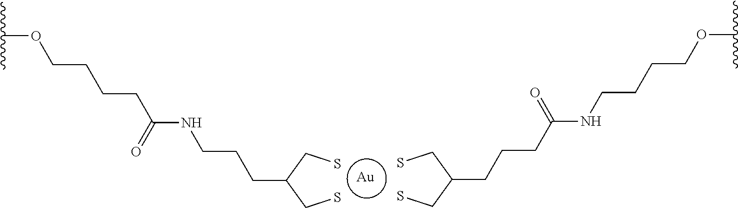 |
| C35 | 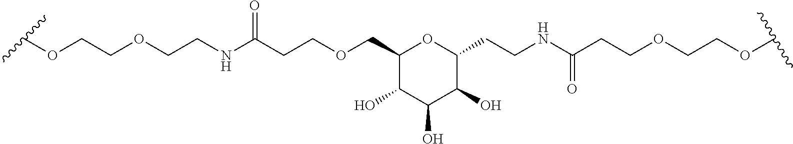 [SMILES: *OCCOCCNC(=O)CCOC[C@H]1O[C@H](CCNC(=O)CCOCCO*)[C@@H](O)[C@@H](O)[C@@H]1O] |
| C36 | 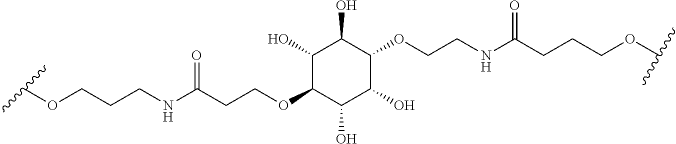 [SMILES: *OCCCNC(=O)CCO[C@H]1[C@H](O)[C@@H](O)[C@H](OCCNC(=O)CCCO*)[C@H](O)[C@@H]1O] |
| C37 | 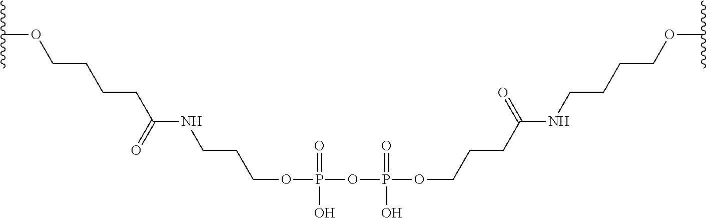 [SMILES: *OCCCCNC(=O)CCCOP(=O)(O)OP(=O)(O)OCCCNC(=O)CCCCO*] |
| C38 | 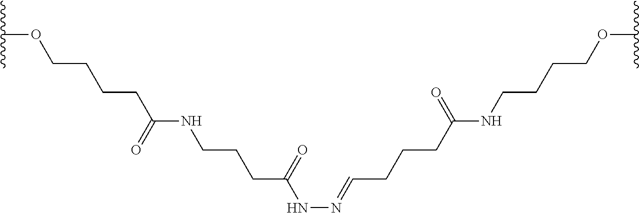 [SMILES: *OCCCCNC(=O)CCC/C=N/NC(=O)CCCNC(=O)CCCCO*] |

TABLE 4

| No. | Structure |
|---|---|
| D1 | 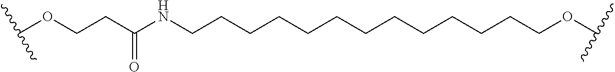 [SMILES: *OCCCCCCCCCCCCCNC(=O)CCO*] |
| D2 | 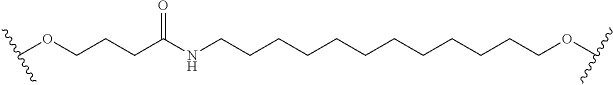 [SMILES: *OCCCCCCCCCCCCNC(=O)CCCO*] |
| D3 | 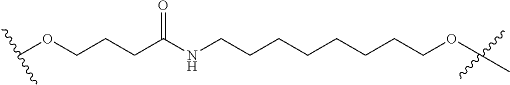 [SMILES: *OCCCCCCCCNC(=O)CCCO*] |
| D4 | 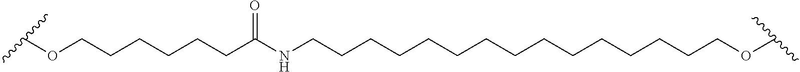 [SMILES: *OCCCCCCCCCCCCCCCNC(=O)CCCCCCO*] |
| D5 | 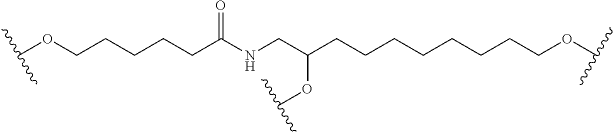 [SMILES: *OCCCCCCCCC(CNC(=O)CCCCCO*)O*] |
| D6 | 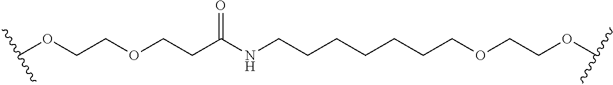 [SMILES: *OCCOCCCCCCCNC(=O)CCOCCO*] |
| D7 | 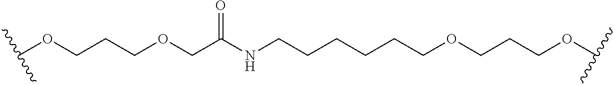 [SMILES: *OCCCOCCCCCCNC(=O)COCCCO*] |
| D8 | 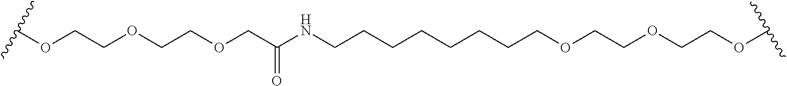 [SMILES: *OCCOCCOCCCCCCCCNC(=O)COCCOCCO*] |
| D9 | 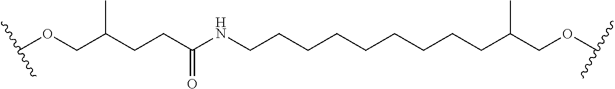 [SMILES: *OCC(C)CCCCCCCCCNC(=O)CCC(C)CO*] |
| D10 | 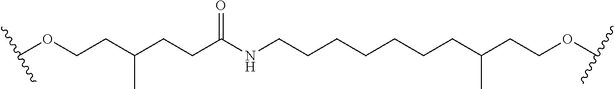 [SMILES: *OCCC(C)CCCCCCCNC(=O)CCC(C)CCO*] |
| D11 | 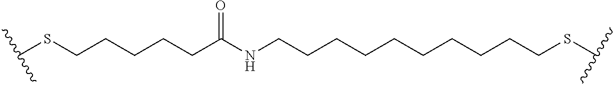 [SMILES: *SCCCCCCCCCCNC(=O)CCCCCS*] |
| D12 | 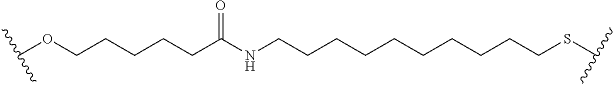 [SMILES: *OCCCCCC(=O)NCCCCCCCCCCS*] |
| D13 | 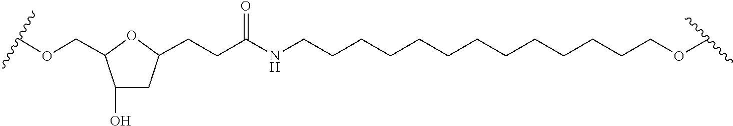 [SMILES: *OCCCCCCCCCCCCCNC(=O)CCC1CC(O)C(CO*)O1] |
| D14 | 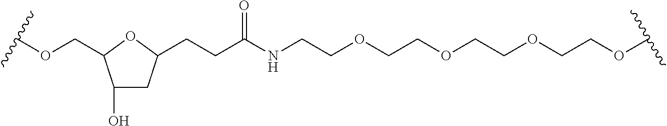 [SMILES: *OCCOCCOCCOCCNC(=O)CCC1CC(O)C(CO*)O1] |
| D15 | 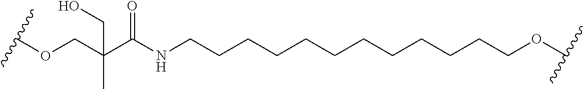 [SMILES: *OCCCCCCCCCCCCNC(=O)C(C)(CO)CO*] |
| D16 | 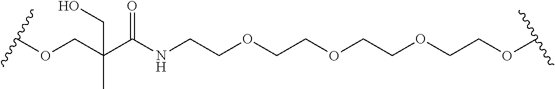 [SMILES: *OCCOCCOCCOCCNC(=O)C(C)(CO)CO*] |
| D17 | 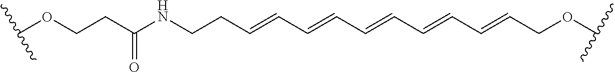 [SMILES: *OC/C=C/C=C/C=C/C=C/C=C/CCNC(=O)CCO*] |
| D18 | 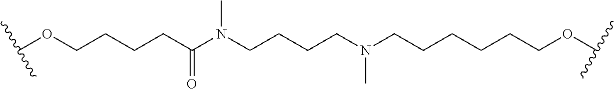 [SMILES: *OCCCCCCN(C)CCCCN(C)C(=O)CCCCO*] |
| D19 | 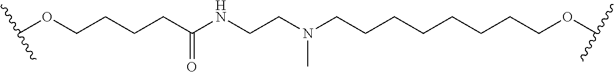 [SMILES: *OCCCCCCCCN(C)CCNC(=O)CCCCO*] |
| D20 | 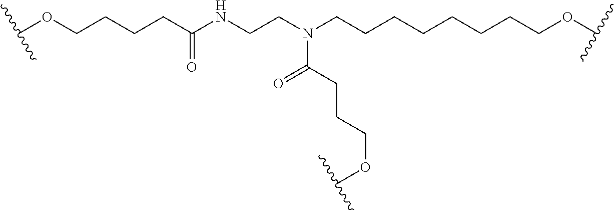 [SMILES: *OCCCCCCCCN(CCNC(=O)CCCCO*)C(=O)CCCO*] |
| D21 | 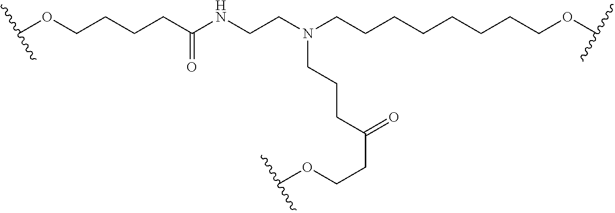 [SMILES: *OCCCCCCCCN(CCCC(=O)CCO*)CCNC(=O)CCCCO*] |
| D22 | 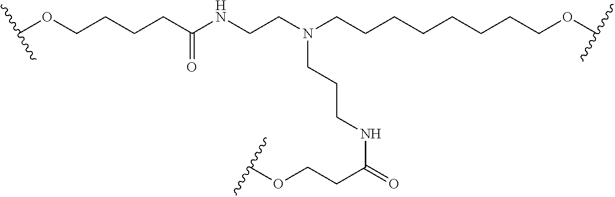 [SMILES: *OCCCCCCCCN(CCCNC(=O)CCO*)CCNC(=O)CCCCO*] |
| D23 | 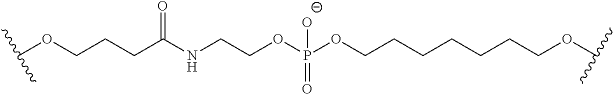 [SMILES: *OCCCCCCCOP(=O)([O-])OCCNC(=O)CCCO*] |
| D24 | 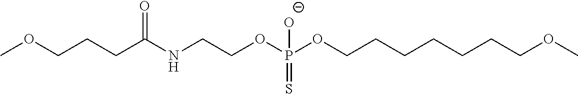 |
| D25 | 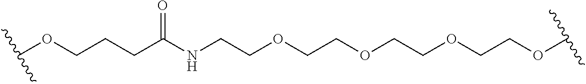 [SMILES: *OCCCC(=O)NCCOCCOCCOCCO*] |
| D26 | 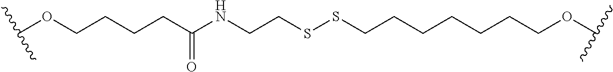 [SMILES: *OCCCCCCCSSCCNC(=O)CCCCO*] |
| D27 | 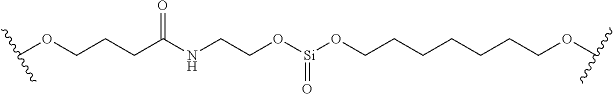 [SMILES: *OCCCCCCCO[Si](=O)OCCNC(=O)CCCO*] |
| D28 | 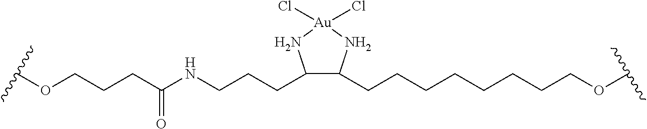 |
| D29 | 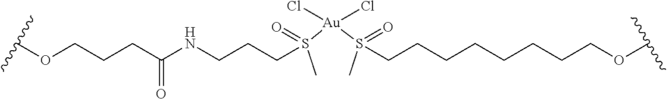 |
| D30 | 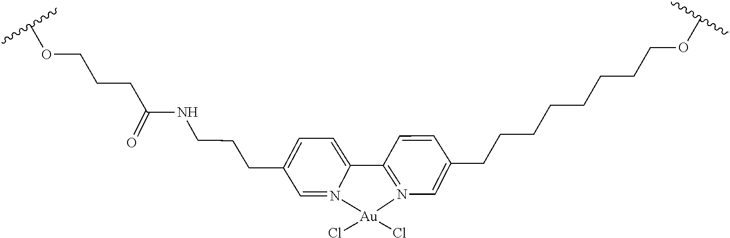 |
| D31 | 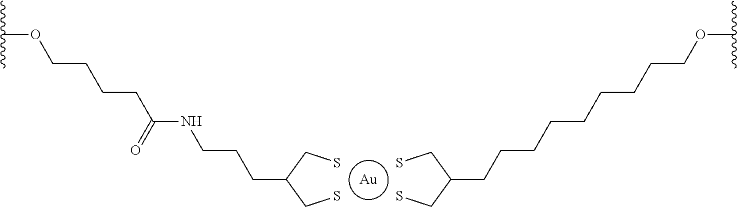 |
| D32 | 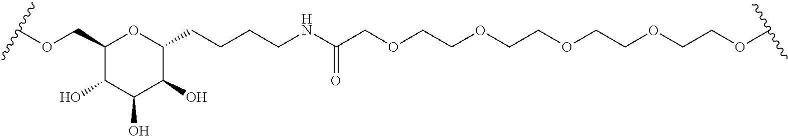 [SMILES: *OCCOCCOCCOCCOCC(=O)NCCCC[C@H]1O[C@H](CO*)[C@@H](O)[C@H](O)[C@@H]1O] |
| D33 | 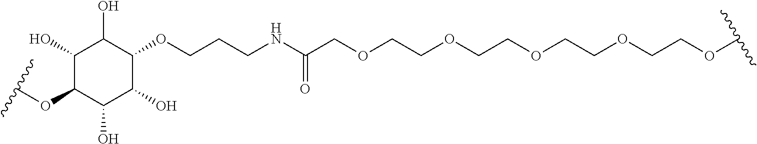 [SMILES: *OCCOCCOCCOCCOCC(=O)NCCCO[C@H]1C(O)[C@@H](O)[C@H](O*)[C@@H](O)[C@H]1O] |
| D34 | 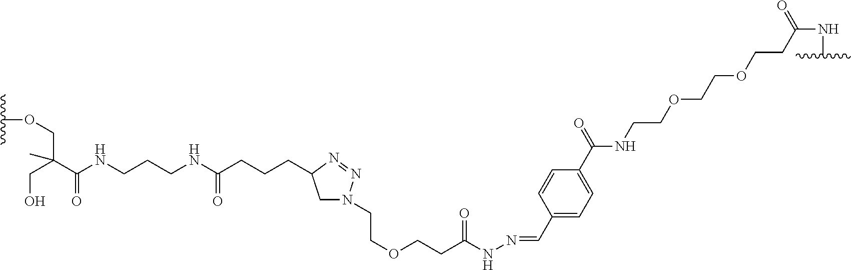 [SMILES: *NC(=O)CCOCCOCCNC(=O)c1ccc(/C=N/NC(=O)CCOCCN2CC(CCCC(=O)NCCCNC(=O)C(C)(CO)CO*)N=N2)cc1] |
| D35 | 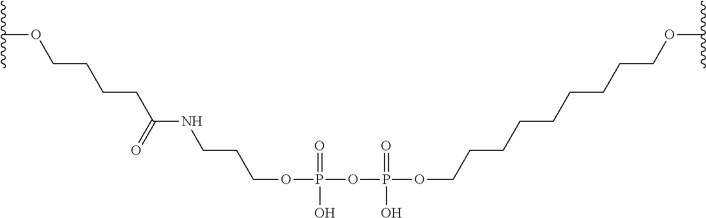 [SMILES: *OCCCCCCCCCOP(=O)(O)OP(=O)(O)OCCCNC(=O)CCCCO*] |
| D36 | 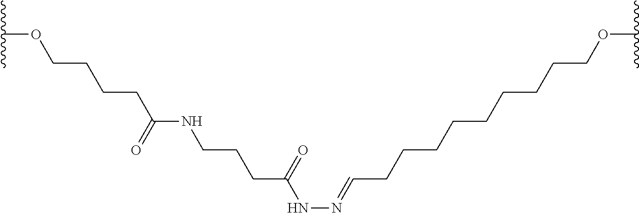 [SMILES: *OCCCCCCCCC/C=N/NC(=O)CCCNC(=O)CCCCO*] |

TABLE 5

| No. | Structure |
|---|---|
| E1 | 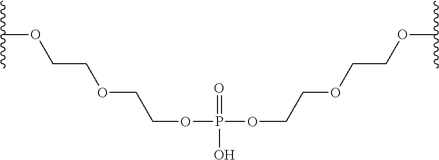 [SMILES: *OCCOCCOP(=O)(O)OCCOCCO*] |
| E2 | 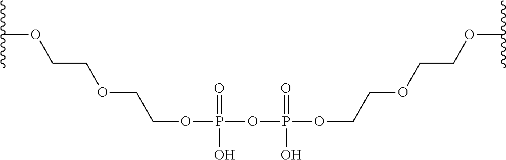 [SMILES: *OCCOCCOP(=O)(O)OP(=O)(O)OCCOCCO*] |
| E3 | 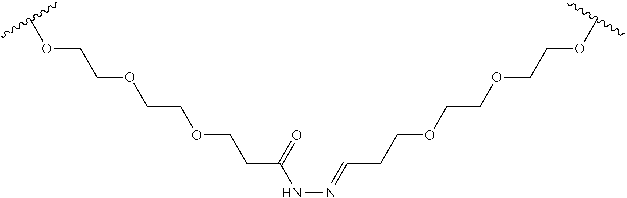 [SMILES: *OCCOCCOCC/C=N/NC(=O)CCOCCOCCO*] |
| E4 | 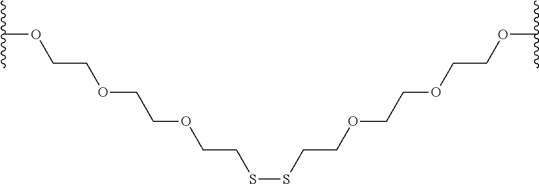 [SMILES: *OCCOCCOCCSSCCOCCOCCO*] |

In Table 1 to Table 5, the wavy lines indicate positions at which the linker is attached to the oligonucleotide and/or the ligand.

In a second aspect, the present disclosure further provides a compound comprising the linker as mentioned above and two or more oligonucleotides covalently attached to the linker.

In an optional embodiment, the two or more oligonucleotides are delivered to a same or a different position of a mRNA or are delivered to two or more different mRNAs.

In an optional embodiment, the linker is attached to a sense strand or an antisense strand of the two or more oligonucleotides; and/or
the linker is attached to a 3′ end and/or a 5′ end of the two or more oligonucleotides.

In an embodiment, the linker is linked (e.g., covelatently attached) to the sense strand of the two or more oligonucleotides.

In an embodiment, the linker is linked (e.g., covelatently attached) to the antisense strand of the two or more oligonucleotides.

In an embodiment, the linker is linked (e.g., covelatently attached) to the sense strand of at least one of the two or more oligonucleotides and to the antisense strand of another oligonucleotide of the two or more oligonucleotides.

Whether the linker is attached to the sense or antisense strand, the linker can be attached to either the 3′ end, the 5′ end or combinations thereof of the two or more oligonucleotides. For example, in one embodiment, the linker is attached to the 3′ ends of the two or more oligonucleotides. In another embodiment, the linker is attached to the 5′ ends of the two or more oligonucleotides. In another embodiment, the linker is attached to the 3′ end of at least one of the two or more oligonucleotides and to the 5′ end of another oligonucleotide of the two or more oligonucleotides.

In an optional embodiment, at least one of the two or more oligonucleotides comprises at least two phosphorothioate linkages within the five nucleotides at the terminus proximal to the linker; and/or
at least one of the two or more oligonucleotides comprises a Ral group at the terminus distal to the linker (inverted abasic); in an embodiment, the linker contains Ral (inverted abasic) at the end of the linker.

In an optional embodiment, the compound has the structure shown in the following structural formulae:

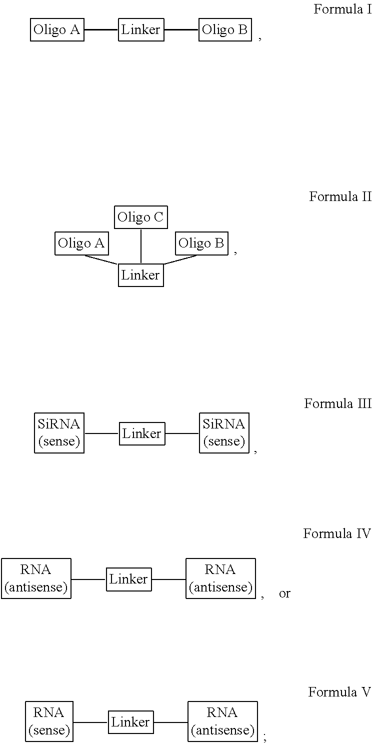

wherein the linker is any linker described herein.

Preferably, the compound has the structure shown in the following structural formulae:

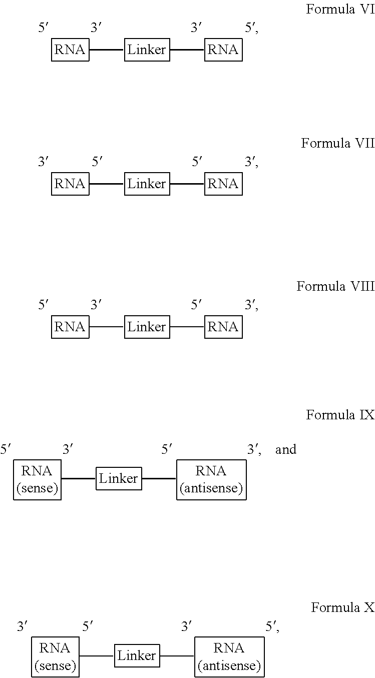

wherein the linker is any linker described herein.

In an optional embodiment, the compound has the structure shown below:

TABLE 6

| Sense strand | Antisense strand |
|---|---|
| 5′-GsUsCAUCCfACfAfAfUGAGAGUACA- | 5′- |
| linker- | UsGfsUACTgnCUCAUUGUGGAUGACs |
| UsCsUCCCAfCCfUfUfUUCUUCUAAU-3′ | GsA-3′(AGT) (see SEQ ID NO. 3 for |
| (see SEQ ID NO. 1 for pre-linker unmodified | unmodified sequence) |
| sequence, see SEQ ID NO. 2 for post-linker | 5′- |
| unmodified sequence) | AsUfsUAGAfAGAAAAGGfUGfGGAGASC |
| | SU-3′ (AGT) (see SEQ ID NO. 4 for |
| | unmodified sequence) |
| 3′-UsGsUUUUCGUUTUGfUCfCAGAsUsC- | 5′- |
| linker- | AsCfsAAfAfAfGCfAAfAAfCAfGGfUCfUA |
| CsUsAGACCfUGfUTUUGCUUUUGU- | GsAsA-3′(see SEQ ID NO. 7 for unmodified |
| 3′(see SEQ ID NO. 5 for pre-linker | sequence) |
| unmodified sequence, | 5′- |
| see SEQ ID NO. 6 for | AsCfsAAfAfAfGCfAAfAAfCAfGGfUCfUA |
| post-linker unmodified sequence) | GsAsA-3′ (see SEQ ID NO. 7 for unmodified |
| | sequence) |
| 3′- | 5′- |
| RalsUsAsUUUACAGAAfAfCfAAfCGUUsC | AsUfsAAAUfGUfCUUUGUUGICAAGCsU |
| fsG-linker- | sUf-3′(see SEQ ID NO. 10 for unmodified |
| GsCfsUUGCAfACfAfAfAGACAUUUAU-3′ | sequence) |
| (see SEQ ID NO. 8 for | 5′- |
| pre-linker unmodified | AsUfsAAAUfGUfCUUUGUfUGfCAAGCsU |
| sequence, see SEQ ID NO. | sUf-3′(see SEQ ID NO. 11 for unmodified |
| 9 for post-linker | sequence) |
| unmodified sequence) | |
| 3′- | 5′- |
| UsAsUCUACAUCAfUfCfUUfAAAGsAsGs- | AsUfsAGfAUfGUAGUAGAfAUfUUCUCs |
| linker- | UsG-3′ (C3) (see SEQ ID NO. 14 for |
| AsAsGfCAfAGfAUfAfUfUUfUUAfUAfAU | unmodified sequence) |
| A-3′ (see SEQ ID NO. 12 for pre-linker | |
| unmodified sequence, see SEQ ID NO. 13 for | |
| post-linker unmodified sequence) | |
| 3′- | 5′- |
| UsGsUGAAAAAACfAfAfAGfUGUfUsUsG | AfsCfsACUUUfUUfUGUUUfCAfCAAACs |
| -linker- | AsA-3′ (AGT) (see SEQ ID NO. 18 for |
| CsUsAGACCfUGfUTUUGCUUUUGU-3′ | unmodified sequence) |
| (see SEQ ID NO. 16 for pre-linker | 5′- |
| unmodified sequence, see SEQ ID NO. 17 for | AsCfsAAfAfAfGCfAAfAAfCAfGGfUCfUA |
| post-linker unmodified sequence) | GsAsA-3′ (PCSK9) (see SEQ ID NO. 19 for |
| | unmodified sequence) |
| 3′- | 5′- |
| RalsUsUsUUUCUGACUfAfGfUUfUAUAsC | AsAfsAAAGfACUGAUCASAAfUAUGUSU |
| sA-linker- | SG-3′ (ANGPTL3) (see SEQ ID NO. 22 for |
| CsUsAGACCfUGfUTUUGCUUUUGU-3′ | unmodified sequence) |
| (see SEQ ID NO. 20 for pre-linker | 5′- |
| unmodified sequence, see SEQ ID NO. 21 for | AsCfsAAfAfAfGCfAAfAAfCAfGGfUCfUA |
| post-linker unmodified sequence) | GsAsA-3′ (PCSK9) (see SEQ ID NO. 23 for |
| | unmodified sequence) |
| 5′-CsUsAGACCfUGfUTUUGCUUUUsGsU- | 5′- |
| linker- | AsCfsAAfAfAfGCfAAfAAfCAfGGfUCfUA |
| UsGsUUUUCGUUTUGfUCfCAGAUC-5′ | GsAsA-3′ (see SEQ ID NO. 7 for unmodified |
| (see SEQ ID NO. 47 for pre-linker | sequence) |
| unmodified sequence, see SEQ ID NO. 47 for | |
| post-linker unmodified sequence) | |
| 5′- | 5′- |
| UsGfsACfUUfUGfAAfCACAfUGfUUfGCf | AfsGsCfAAfCAfUGfUfGfUUfCAfAAfGUfs |
| UsUsU-linker- | CsAf-3′ (CFB) (see SEQ ID NO. 30 for |
| CsUsAGACCfUGfUTUUGCUUUUGU-3′ | unmodified sequence) |
| (see SEQ ID NO. 28 for pre-linker | 5′- |
| unmodified sequence, see SEQ ID NO. 29 for | AsCfsAAfAfAfGCfAAfAAfCAfGGfUCfUA |
| post-linker unmodified sequence) | GsAsA-3′ (PCSK9) (see SEQ ID NO. 31 for |
| | unmodified sequence) |
| 3′-UAAUCUUCUUUfUfCfCAfCCCUsCsU- | 5′- |
| linker- | AsUfsUAGAfAGAAAAGGfUGfGGAGASC |
| CsUsAGACCfUGfUTUUGCUUUUGU-3′- | SU-3′ (AGT) (see SEQ ID NO. 34 for |
| ligand (see SEQ ID NO. 32 for pre-UsUUsU | unmodified sequence) |
| unmodified sequence, see SEQ ID NO. 33 for | |
| post-UsUUsU unmodified sequence) | |
| 3′-UsGsUUUUCGUUTUGfUCfCAGAsUsC- | 5′- |
| linker- | AsCfsAAfAfAfGCfAAfAAfCAfGGfUCfUA |
| CsUsAGACCfUGfUTUUGCUUUUGU-3′ | GsAsA-3′ (see SEQ ID NO. 40 for |
| (see SEQ ID NO. 38 for pre-linker | unmodified sequence) |
| unmodified sequence, see SEQ ID NO. 39 for | |
| post-linker unmodified sequence) | |
| 3′- | 5′- |
| AfsCsUfGAfAAfCUfUGfUfGfUAfCAfACfs | UsGfsACfUUfUGfAAfCACAfUGfUUfGCf |
| GsAf-linker- | UsUsU-3′ (CFB) (see SEQ ID NO. 43 for |
| CsUsAGACCfUGfUTUUGCUUUUGU-3′ | unmodified sequence) |
| (see SEQ ID NO. 41 for pre-A2 unmodified | |
| sequence, see SEQ ID NO. 42 for post-A2 | |
| unmodified sequence) | |
| 3′- | 5′- |
| RalsAfsCsUfGAfAAfCUfUGfUfGfUAfCAf | UsGfsACfUUfUGfAAfCACAfUGfUUfGCf |
| ACfsGsAf-linker- | UsUsU-3′ (CFB) (see SEQ ID NO. 46 for |
| CsUsAGACCfUGfUTUUGCUUUUGU-3′ | unmodified sequence) |
| (see SEQ ID NO. 44 for pre-A2 unmodified | |
| sequence, see SEQ ID NO. 45 for post-A2 | |
| unmodified sequence) | |

wherein the linker is any linker described herein; the lowercase f represents that an adjacent nucleotide on the left side of the letter f is a 2′-fluoro modified nucleotide; and the lowercase s represents that two adjacent nucleotides on the left and right sides of the letter s are linked by a phosphorothioate linkage.

In an optional embodiment, the compound has the structure shown below:

TABLE 7

| | Sense strand | Antisense strand |
|---|---|---|
| SNK- | 5′- | 5′- |
| 3464′ | GsUsCAUCCfACfAfAfUGAGAGUA | UsGfsUACTgnCUCAUUGUfGGfAU |
| | CA-A2- | GACsGsA-3′(AGT) (see SEQ ID NO. 3 |
| | UsCsUCCCAfCCfUfUfUUCUUCUA | for unmodified sequence) |
| | AU-3′ (see SEQ ID NO. 1 for pre-A2 | 5′- |
| | unmodified sequence, see SEQ ID NO. | AsUfsUAGAfAGAAAAGGfUGfGGA |
| | 2 for post-A2 unmodified sequence) | GAsCsU-3′ (AGT) (see SEQ ID NO. 4 |
| | | for unmodified sequence) |
| SNK- | 3′- | 5′- |
| 2998′ | RalsUsAsUUUACAGAAfAfCfAAfCG | AsUfsAAAUfGUfCUUUGUfUGfCAA |
| | UUsCfsG-A2- | GCsUsUf-3′ (see SEQ ID NO. 10 for |
| | GsCfsUUGCAfACfAfAfAGACAUUU | unmodified sequence) |
| | AU-3′ (see SEQ ID NO. 8 for pre-A2 | 5′- |
| | unmodified sequence, see SEQ ID NO. | AsUfsAAAUfGUfCUUUGUfUGfCAA |
| | 9 for post-A2 unmodified sequence) | GCsUsUf-3′ (see SEQ ID NO. 11 for |
| | | unmodified sequence) |
| SNK- | 3′-UsAsUCUACAUCAfUfCfUUf | 5′-AsUfsAGfAUfGUAG |
| 3467′ | AAAGsAsGs-A2-AsAsGfCAfAG | UAGAfAUfUUCUCsUsG-3′ (C3) (see |
| | fAUfAfUfUUfUUAfUAfAUA-3′ (see | SEQ ID NO. 14 for unmodified |
| | SEQ ID NO. 12 for pre-A2 unmodified | sequence) |
| | sequence, see SEQ ID NO. 13 for post- | 5′-UsAfsUfUAfUAAfAAfAUAU |
| | A2 unmodified sequence) | fCUfUGfCUUsUsU-3′ (C5) (see SEQ |
| | | ID NO. 15 for unmodified sequence) |
| SNK- | 3′- | 5′-AfsCfsACUUUfUUfUG |
| 3010′ | UsGsUGAAAAAACfAfAfAGfUGUf | UUUfCAfCAAACsAsA-3′ (AGT) (see |
| | UsUsG-A2- | SEQ ID NO. 18 for unmodified |
| | CsUsAGACCfUGfUTUUGCUUUUG | sequence) |
| | U-3′ (see SEQ ID NO. 16 for pre-A2 | 5′-AsCfsAAfAfAfGCfAAfAAf |
| | unmodified sequence, see SEQ ID NO. | CAfGGfUCfUAGsAsA-3′ (PCSK9) |
| | 17 for post-A2 unmodified sequence) | (see SEQ ID NO. 19 for unmodified |
| | | sequence) |
| SNK- | 3′- | 5′- |
| 3005′ | RalsUsUsUUUCUGACUfAfGfUUfUA | AsAfsAAAGfACUGAUCASAAfUAU |
| | UAsCsA-A2- | GUsUsG-3′ (ANGPTL3) (see SEQ ID |
| | CsUsAGACCfUGfUTUUGCUUUUG | NO. 22 for unmodified sequence) |
| | U-3′ (see SEQ ID NO. 20 for pre-A2 | 5′- |
| | unmodified sequence, see SEQ ID NO. | AsCfsAAfAfAfGCfAAfAAfCAfGGfU |
| | 21 for post-A2 unmodified sequence) | CfUAGsAsA-3′ (PCSK9) (see SEQ ID |
| | | NO. 23 for unmodified sequence) |
| SNK- | 3′- | 5′- |
| 3461′ | RalsUsUsUUUCUGACUfAfGfUUfUA | AsAfsAAAGfACUGAUCASAAfUAU |
| | UAsCsA-A4- | GUsUsG-3′ (ANGPTL3) (see SEQ ID |
| | CsUsAGACCfUGfUTUUGCUUUUG | NO. 26 for unmodified sequence) |
| | U-3′ (see SEQ ID NO. 24 for pre-A4 | 5′- |
| | unmodified sequence, see SEQ ID NO. | AsCfsAAfAfAfGCfAAfAAfCAfGGfU |
| | 25 for post-A4 unmodified sequence) | CfUAGsAsA-3′ (PCSK9) (see SEQ ID |
| | | NO. 27 for unmodified sequence) |
| SNK- | 5′- | 5′- |
| 3008′ | CsUsAGACCfUGfUTUUGCUUUUsG | AsCfsAAfAfAfGCfAAfAAfCAfGGfU |
| | SU-A7- | CfUAGsAsA-3′ (see SEQ ID NO. 7 for |
| | UsGsUUUUCGUUTUGfUCfCAGAU | unmodified sequence) |
| | C-5′ (see SEQ ID NO. 47 for pre-A7 | 5′- |
| | unmodified sequence, see SEQ ID NO. | AsCfsAAfAfAfGCfAAfAAfCAfGGfU |
| | 48 for post-A7 unmodified sequence) | CfUAGsAsA-3′ (see SEQ ID NO. 7 for |
| | | unmodified sequence) |
| SNK- | 5′- | 5′- |
| 3468′ | UsGfsACfUUfUGfAAfCACAfUGfUU | AfsGsCfAAfCAfUGfUfGfUUfCAfAA |
| | fGCfUsUsU-A2- | fGUfsCsAf-3′ (CFB) (see SEQ ID NO. |
| | CsUsAGACCfUGfUTUUGCUUUUG | 30 for unmodified sequence) |
| | U-3′ (see SEQ ID NO. 28 for pre-A2 | 5′- |
| | unmodified sequence, see SEQ ID NO. | AsCfsAAfAfAfGCfAAfAAfCAfGGfU |
| | 29 for post-A2 unmodified sequence) | CfUAGsAsA-3′ (PCSK9) (see SEQ ID |
| | | NO. 31 for unmodified sequence) |
| SNK- | 3′- | 5′- |
| 2192′ | UAAUCUUCUUUfUfCfCAfCCCUsC | AsUfsUAGAfAGAAAAGGfUGfGGA |
| | SU-UsUUsU- | GAsCsU-3′ (AGT) (see SEQ ID NO. 34 |
| | CsUsAGACCfUGfUTUUGCUUUUG | for unmodified sequence) |
| | U-3′ (see SEQ ID NO. 32 for pre- | 5′- |
| | UsUUsU unmodified sequence, see | AsCfsAAfAfAfGCfAAfAAfCAfGGfU |
| | SEQ ID NO. 33 for post-UsUUsU | CfUAGsAsA-3′ (PCSK9) (see SEQ ID |
| | unmodified sequence) | NO. 31 for unmodified sequence) |
| SNK- | 3′- | 5′- |
| 3466′ | UAAUCUUCUUUfUfCfCAfCCCUsC | AsUfsUAGAfAGAAAAGGfUGfGGA |
| | SU-A2- | GAsCsU-3′ (AGT) (see SEQ ID NO. 37 |
| | CsUsAGACCfUGfUTUUGCUUUUG | for unmodified sequence) |
| | U-3′ (see SEQ ID NO. 35 for pre-A2 | 5′- |
| | unmodified sequence, see SEQ ID NO. | AsCfsAAfAfAfGCfAAfAAfCAfGGfU |
| | 36 for post-A2 unmodified sequence) | CfUAGsAsA-3′ (PCSK9) (see SEQ ID |
| | | NO. 31 for unmodified sequence) |
| SNK- | 3′- | 5′- |
| 2959′ | UsGsUUUUCGUUTUGfUCfCAGASU | AsCfsAAfAfAfGCfAAfAAfCAfGGfU |
| | sC-linker- | CfUAGsAsA-3′ (see SEQ ID NO. 40 |
| | CsUsAGACCfUGfUTUUGCUUUUG | for unmodified sequence) |
| | U-3′ (see SEQ ID NO. 38 for pre-linker | 5′- |
| | unmodified sequence, see SEQ ID NO. | AsCfsAAfAfAfGCfAAfAAfCAfGGfU |
| | 39 for post-linker unmodified | CfUAGsAsA-3′ (see SEQ ID NO. 40 |
| | sequence) | for unmodified sequence) |
| SNK- | 3′- | 5′- |
| 2960′ | UsGsUUUUCGUUTUGfUCfCAGAsU | AsCfsAAfAfAfGCfAAfAAfCAfGGfU |
| | sC-A2- | CfUAGsAsA-3′ (see SEQ ID NO. 7 for |
| | CsUsAGACCfUGfUTUUGCUUUUG | unmodified sequence) |
| | U-3′(see SEQ ID NO. 5 for pre-A2 | 5′- |
| | unmodified sequence, see SEQ ID NO. | AsCfsAAfAfAfGCfAAfAAfCAfGGfU |
| | 6 for post-A2 unmodified sequence) | CfUAGsAsA-3′ (see SEQ ID NO. 7 for |
| | | unmodified sequence) |
| SNK- | 3′- | 5′- |
| 3469′ | AfsCsUfGAfAAfCUfUGfUfGfUAfCA | UsGfsACfUUfUGfAAfCACAfUGfUU |
| | fACfsGsAf-A2- | fGCfUsUsU-3′ (CFB) (see SEQ ID |
| | CsUsAGACCfUGfUTUUGCUUUUG | NO. 43 for unmodified sequence) |
| | U-3′ (see SEQ ID NO. 41 for pre-A2 | 5′- |
| | unmodified sequence, see SEQ ID NO. | AsCfsAAfAfAfGCfAAfAAfCAfGGfU |
| | 42 for post-A2 unmodified sequence) | CfUAGsAsA-3′ (PCSK9) (see SEQ ID |
| | | NO. 7 for unmodified sequence) |
| SNK- | 3′- | 5′- |
| 3470′ | RalsAfsCsUfGAfAAfCUfUGfUfGfUA | UsGfsACfUUfUGfAAfCACAfUGfUU |
| | fCAfACfsGsAf-A2- | fGCfUsUsU-3′ (CFB) (see SEQ ID |
| | CsUsAGACCfUGfUTUUGCUUUUG | NO. 46 for unmodified sequence) |
| | U-3′ (see SEQ ID NO. 44 for pre-A2 | 5′- |
| | unmodified sequence, see SEQ ID NO. | AsCfsAAfAfAfGCfAAfAAfCAfGGfU |
| | 45 for post-A2 unmodified sequence) | CfUAGsAsA-3′ (PCSK9) (see SEQ ID |
| | | NO. 7 for unmodified sequence) |

wherein the linker is any linker described herein; the lowercase f represents that an adjacent nucleotide on the left side of the letter f is a 2′-fluoro modified nucleotide; the lowercase s represents that two adjacent nucleotides on the left and right sides of the letter s are linked by a phosphorothioate linkage; A2 is a linker with No. A2; A4 is a linker with No. A4; A7 is a linker with No. A7; and Tgn is Thymidine glycol nucleic acid.

In an optional embodiment, the present disclosure further provides the compound as mentioned above, further comprising a ligand covalently attached to the linker and/or one of the two or more oligonucleotides.

In an optional embodiment, the present disclosure further provides the compound as mentioned above, wherein the ligand is attached to a sense strand or an antisense strand of the two or more oligonucleotides, and/or,
the ligand is attached to a 3′ end and/or a 5′ end of the two or more oligonucleotides.

In an optional embodiment, the compound has the structure shown in the following structural formulae:

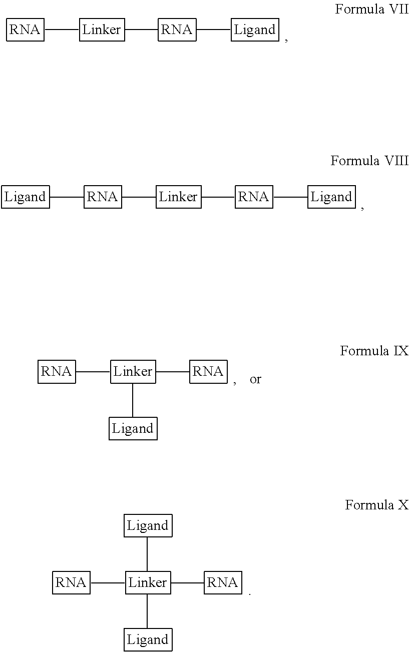

In an optional embodiment, the ligand has the structure shown in the following structural formulae:

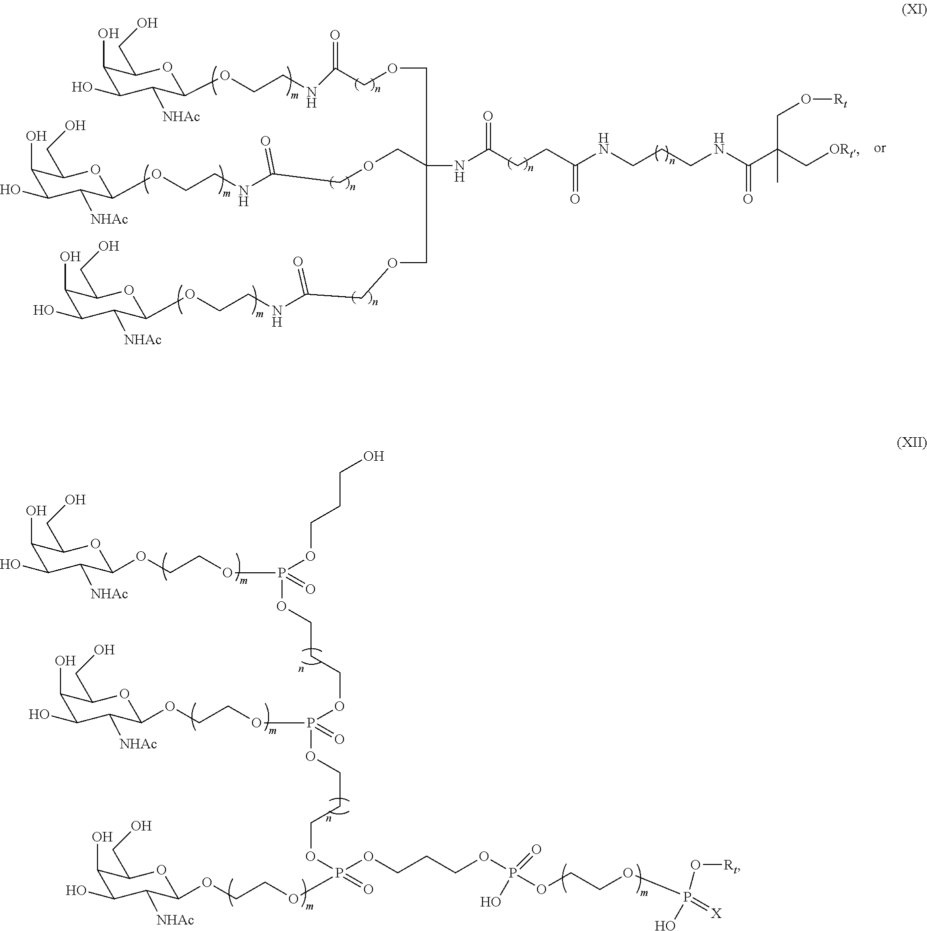

wherein m and n on formula (XI) or formula (XII) are each independently an integer of 0-6; Rt and
Rt′ are each independently selected from H, hydroxyl protecting group, oligonucleotide, linkers described above, the compound described in the second aspect of the present disclosure, solid phase carrier, C₂-C₆ carboxyl, carboxylate or amide group;
wherein the hydroxyl protecting group is one selected from the group consisting of 4,4′-dimethoxytrityl, 4-methoxytrityl, trityl, t-butyldimethylsilyl, 4-oxopentanoyl, 2-cyanoethyl, 4-pentenoyl and acyloxyalkyl group;
preferably, Rt is oligonucleotide, linkers described above, or the compound described in the second aspect of the present disclosure.

In an optional embodiment, the ligand has the structure shown in the following structural formulae:

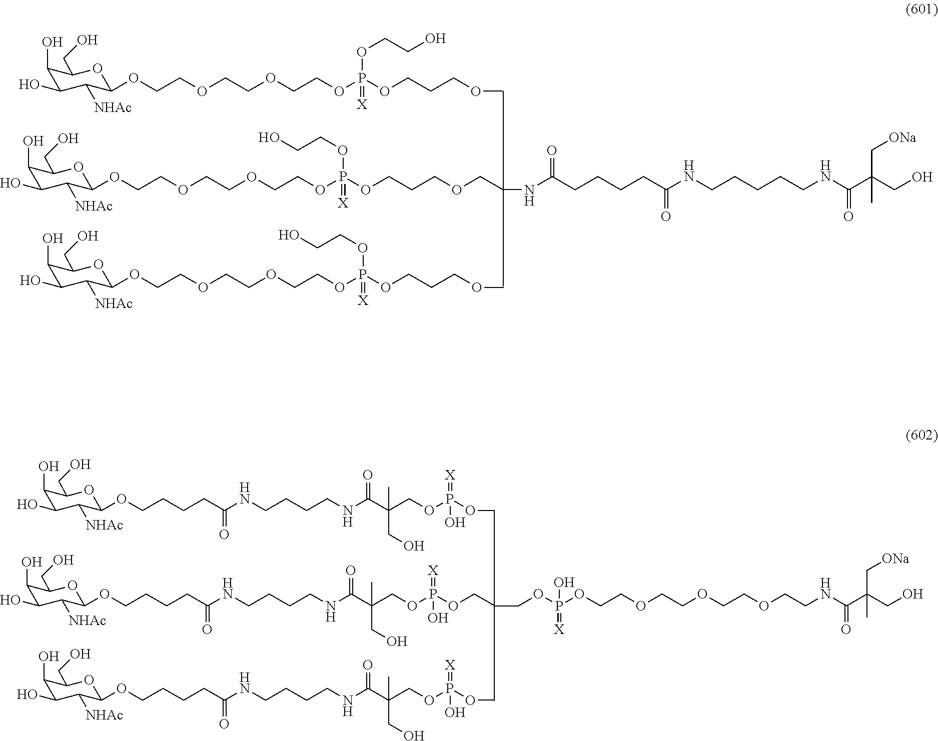

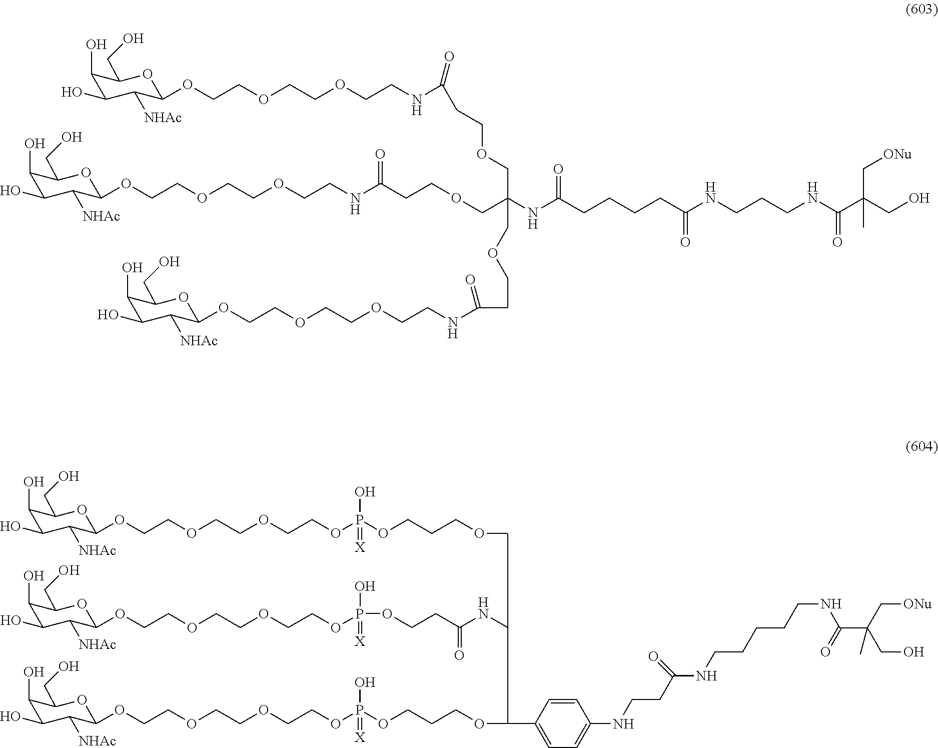

wherein X on formula (601), (602), (603), (604), (605), (606) or (607) is O or S; and Nu is a compound according to any aspect of the invention as described herein.

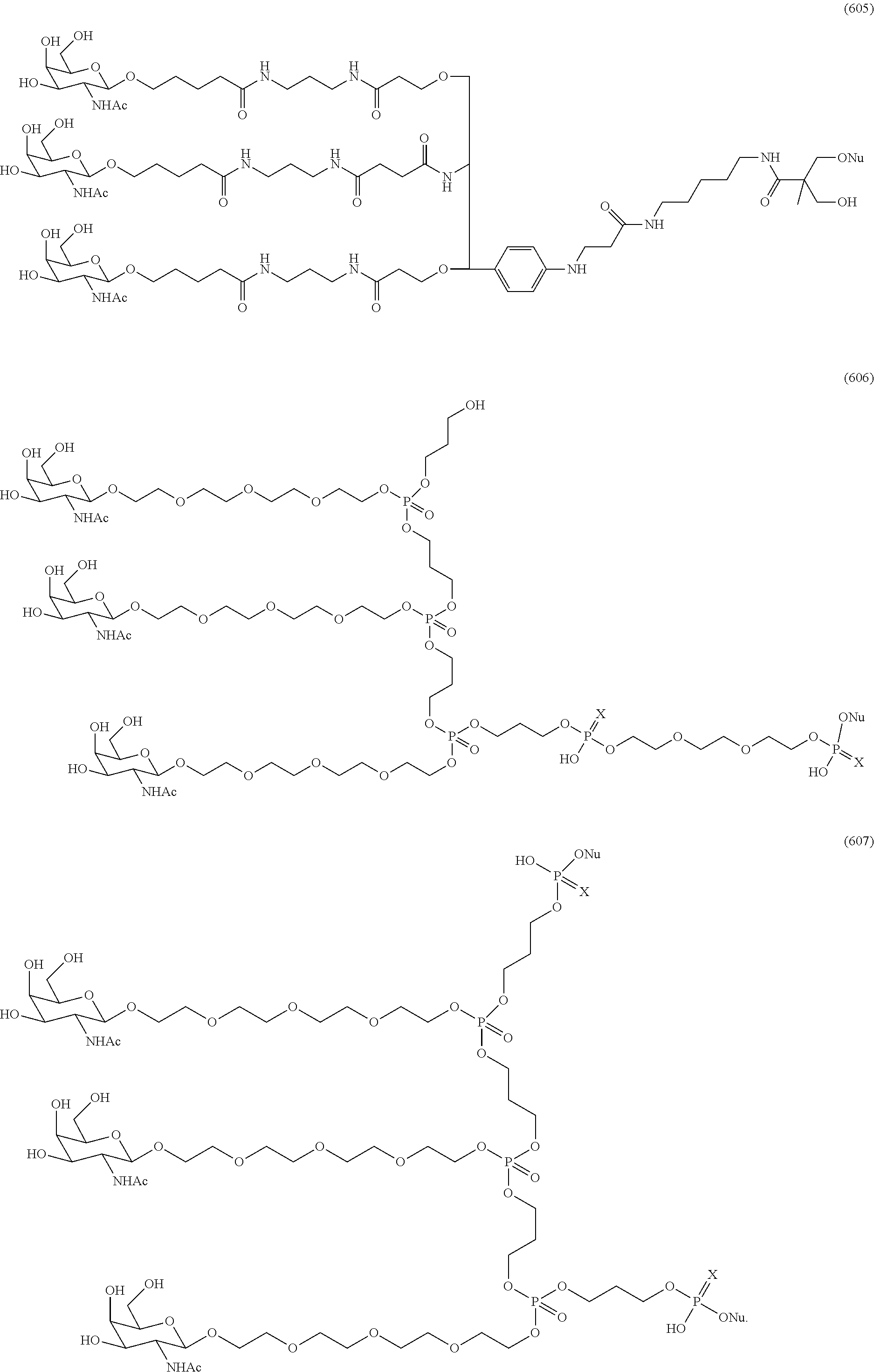

In an optional embodiment, the compound has the structure shown below:

TABLE 8

| Sense strand | Antisense strand |
|---|---|
| 5′- | 5′- |
| GsUsCAUCCfACfAfAfUGAGAGUACA- | UsGfsUACTgnCUCAUUGUfGGfAUGACsGs |
| linker- | A-3′(AGT) (see SEQ ID NO. 3 for unmodified |
| UsCsUCCCAfCCfUfUfUUCUUCUAAU-3′ | sequence) |
| -ligand (see SEQ ID NO. 1 for pre-linker | 5′- |
| unmodified sequence, see SEQ ID NO. 2 | AsUfsUAGAfAGAAAAGGfUGfGGAGAsCsU-3′ |
| for post-linker unmodified sequence) | (AGT) (see SEQ ID NO. 4 for unmodified |
| | sequence) |

5′-GsUsCAUCCfACfAfAfUGAGAGUACA-3′-linker-5′-UsCsUCCCAfCCfUfUfUUCUUCUAAU-3′-ligand
3′-AsGsCAGUAGfGUfGUUACUCTgnCAUsGfsU-5′ 3′-UsCsAGAGGGfUGfGAAAAGAAfGAUsUfsa-5′

| | |
|---|---|
| 3′- | 5′- |
| UsGsUUUUCGUUTUGfUCfCAGAsUsC- | AsCfsAAfAfAfGCfAAfAAfCAfGGfUCfUAGsAsA-3′ |
| linker- | (see SEQ ID NO. 7 for unmodified |
| CsUsAGACCfUGfUTUUGCUUUUGU- | sequence) |
| 3′-ligand (see SEQ ID NO. 5 for pre-linker | 5′- |
| unmodified sequence, see SEQ ID NO. 6 | AsCfsAAfAfAfGCfAAfAAfCAfGGfUCfUAGsAsA-3′ |
| for post-linker unmodified sequence) | (see SEQ ID NO. 7 for unmodified |
| | sequence) |

3′-UsGsUUUUCGUUTUGfUCfCAGAsUsC-5′-linker-5′CsUsAGACCfUGfUTUUGCUUUUGU-3′-ligand
5′-AsCfsAAfAfAfGCfAAfAAfCAfGGfUCfUAGsAsA-3′ 3′-AsAsGAUCfUGfGfCAfAAfACfGAfAfAsCfsA-5′

| | |
|---|---|
| 3′- | 5′- |
| RalsUsAsUUUACAGAAfAfCfAAfCGU | AsUfsAAAUfGUfCUUUGUfUGfCAAGCsUsUf-3′ |
| UsCfsG-linker- | (see SEQ ID NO. 10 for unmodified |
| GsCfsUUGCAfACfAfAfAGACAUUUAU | sequence) |
| -3′-ligand (see SEQ ID NO. 8 for pre- | 5′- |
| linker unmodified sequence, see SEQ ID | AsUfsAAAUfGUfCUUUGUfUGfCAAGCsUs |
| NO. 9 for post-linker unmodified | Uf-3′ (see SEQ ID NO. 11 for unmodified |
| sequence) | sequence) |

3′-RalsUsAsUUUACAGAAfAfCfAAfCGUUsCfsG-5′-linker-5′-GsCfsUUGCAfACfAfAfAGACAUUUAU-3′-ligand
5′-AsUfsAAAUfGUfCUUUGUfUGfCAAGCsUsUf-3′ 3′ UfUsCGAACGfUUfGUUUCUfGUfAAAsUfsA-5′

| | |
|---|---|
| 3′- | 5′- |
| UsAsUCUACAUCAfUfCfUUfAAAGsAs | AsUfsAGfAUfGUAGUAGAfAUfUUCUCsUsG-3′ |
| Gs-linker- | (C3) (C3) (see SEQ ID NO. 14 for |
| AsAsGfCAfAGfAUfAfUfUUfUUAfUAf | unmodified sequence) |
| AUA-3′-TriGalNAc (see SEQ ID NO. 12 | |
| for pre-linker unmodified sequence, see | |
| SEQ ID NO. 13 for post-linker unmodified | |
| sequence) | |

3′-UsAsUCUACAUCAfUfCfUUfAAAGsAsGs-5′-linker-5′-AsAsGfCAfAGfAUfAfUfUUfUUAfUAfAUA-3′
5′-AsUfsAGfAUfGUAGUAGAfAUfUUCUCsUsG-3′ 3′-UsUsUUCGfUUfAUAAfAAfAUAfUUfsAfsU-5′

| | |
|---|---|
| 3′- | 5′- |
| UsGsUGAAAAAACfAfAfAGfUGUfUSU | AfsCfsACUUUfUUfUGUUUfCAfCAAACsAs |
| sG-linker- | A-3′ (AGT) (see SEQ ID NO. 18 for |
| CsUsAGACCfUGfUTUUGCUUUUGU- | unmodified sequence) |
| 3′-ligand (see SEQ ID NO. 16 for pre- | 5′- |
| linker unmodified sequence, see SEQ ID | AsCfsAAfAfAfGCfAAfAAfCAfGGfUCfUAG |
| NO. 17 for post-linker unmodified | SASA-3′ (PCSK9) (see SEQ ID NO. 19 for |
| sequence) | unmodified sequence) |

3′-UsGsUGAAAAAACfAfAfAGfUGUfUSUsG-5′-linker-5′-CsUsAGACCfUGfUTUUGCUUUUGU-3′-ligand
5′-AfsCfsACUUUfUUfUGUUUfCAfCAAACsAsA-3′ 3′-AsAsGAUCfUGfGAfCAfAAfACfGAfAfAfAsCfsA-5′

| | |
|---|---|
| 3′- | 5′- |
| RalsUsUsUUUCUGACUfAfGfUUfUAU | AsAfsAAAGfACUGAUCASAAfUAUGUsUs |
| AsCsA-linker- | G-3′ (ANGPTL3) (see SEQ ID NO. 22 for |
| CsUsAGACCfUGfUTUUGCUUUUGU- | unmodified sequence) |
| 3′-ligand (see SEQ ID NO. 20 for pre- | 5′- |
| linker unmodified sequence, see SEQ ID | AsCfsAAfAfAfGCfAAfAAfCAfGGfUCfUAG |
| NO. 21 for post-linker unmodified | sAsA-3′ (PCSK9) (see SEQ ID NO. 23 for |
| sequence) | unmodified sequence) |

```
      3′-RalsUsUsUUUCUGACUfAfGfUUfUAUAsCsA-5′-linker-5′-CsUsAGACCfUGfUTUUGCUUUUGU-3′-ligand
                  5′-AsAfsAAAGfACUGAUCASAAfUAUGUsUsG-3′ 3′-AsAsGAUCfUGfGAfCAfAAfACfGAfAfAfAsCfsA-5′
5′-CsUsAGACCfUGAUfUUGCUUUUsGsU-3′-linker-3′-UsGsUUUUCGUUfUGfUCfCAGAUC-5′
      3′-AsAsGAUCfUGfGAfCAfAAfACfGAfAfAfAsCfsA-5′ | 5′-AsCfsAAfAfAfGCfAAfAAfCAfGGfUCfUAGsAsA-3′
    ligand
```

| | |
|---|---|
| 5′- | 5′- |
| UsGfsACfUUfUGfAAfCACAfUGfUUfG | AfsGsCfAAfCAfUGfUfGfUUfCAfAAfGUfsC |
| CfUsUsU-linker- | sAf-3′ (CFB) (see SEQ ID NO. 30 for |
| CsUsAGACCfUGfUTUUGCUUUUGU- | unmodified sequence) |
| 3′-ligand (see SEQ ID NO. 28 for pre- | 5′- |
| linker unmodified sequence, see SEQ ID | AsCfsAAfAfAfGCfAAfAAfCAfGGfUCfUAG |
| NO. 29 for post-linker unmodified | sAsA-3′ (PCSK9) (see SEQ ID NO. 31 for |
| sequence) | unmodified sequence) |

3′-AsAsGAUCfUGfGAfCAfAAfACfGAfAfAfAsCfsA-5′
5′-UsGfsACfUUfUGfAAfCACAfUGfUUfGCfUsUsU-3′-linker-5′-CsUsAGACCfUGfUTUUGCUUUUGU-3′-ligand
3′-AfsCsUfGAfAAfCUfUGfUfGfUAfCAfACfsGsAf-5′

| | |
|---|---|
| 3′- | 5′- |
| UAAUCUUCUUUfUfCfCAfCCCUsCsU- | AsUfsUAGAfAGAAAAGGIUGIGGAGAsCsU-3′ |
| linker- | (AGT) (see SEQ ID NO. 34 for |
| CsUsAGACCfUGfUTUUGCUUUUGU- | unmodified sequence) |
| 3′-ligand (see SEQ ID NO. 32 for pre- | |
| linker unmodified sequence, see SEQ ID | |
| NO. 33 for post-linker unmodified | |
| sequence) | |

3′-UAAUCUUCUUUfUfCfCAfCCCUsCsU-5′-linker-5′-CsUsAGACCfUGfUTUUGCUUUUGU-3′-ligand
5′-AsUfsUAGAfAGAAAAGGIUGIGGAGAsCsU-3′ 3′-AsCfsAAfAfAfGCfAAfAAfCAfGGfUCfUAGsAsA-5′

| | |
|---|---|
| 3′- | 5′- |
| UsGsUUUUCGUUTUGfUCfCAGAsUsC- | AsCfsAAfAfAfGCfAAfAAfCAfGGfUCfUAG |
| linker- | sAsA-3′ (see SEQ ID NO. 40 for unmodified |
| CsUsAGACCfUGfUTUUGCUUUUGU- | sequence) |
| 3′-ligand (see SEQ ID NO. 38 for pre- | |
| linker unmodified sequence, see SEQ ID | |
| NO. 39 for post-linker unmodified | |
| sequence) | |

3′-UsGsUUUUCGUUTUGfUCfCAGAsUsC-5′-linker-5′-CsUsAGACCfUGfUTUUGCUUUUGU-3′-ligand
5′-AsCfsAAfAfAfGCfAAfAAfCAfGGfUCfUAGsAsA-3′ 3′-AsAsGAUCfUGfGAfCAfAAfACfGAfAfAfAsCfsA-5′

| | |
|---|---|
| 3′- | 5′- |
| AfsCsUfGAfAAfCUfUGfUfGfUAfCAfA | UsGfsACfUUfUGfAAfCACAfUGfUUfGCfUs |
| CfsGsAf-linker- | UsU-3′ (CFB) (see SEQ ID NO. 43 for |
| CsUsAGACCfUGfUTUUGCUUUUGU- | unmodified sequence) |
| 3′-TriGalNAc (see SEQ ID NO. 41 for pre- | |
| A2 unmodified sequence, see SEQ ID NO. | |
| 42 for post-A2 unmodified sequence) | |

3′-AfsCsUfGAfAAfCUfUGfUfGfUAfCAfACfsGsAf-5′-linker-5′-CsUsAGACCfUGfUTUUGCUUUUGU-3′-ligand
5′-UsGfsACfUUfUGfAAfCACAfUGfUUfGCfUsUsU-3′ 3′-AsAsGAUCfUGfGAfCAfAAfACfGAfAfAfAsCfsA-5′

| | |
|---|---|
| 3′- | 5′- |
| RalsAfsCsUfGAfAAfCUfUGfUfGfUAfC | UsGfsACfUUfUGfAAfCACAfUGfUUfGCfUs |
| AfACfsGsAf-linker- | UsU-3′ (CFB) (see SEQ ID NO. 46 for |
| CsUsAGACCfUGfUTUUGCUUUUGU- | unmodified sequence) |
| 3′-TriGalNAc (see SEQ ID NO. 44 for pre- | |
| A2 unmodified sequence, see SEQ ID NO. | |
| 45 for post-A2 unmodified sequence) | |

3′-RalsAfsCsUfGAfAAfCUfUGfUfGfUAfC-5′AfACfsGsAf-linker-5′-CsUsAGACCfUGfUTUUGCUUUUGU-3′-ligand
5′-UsGfsACfUUfUGfAAfCACAfUGfUUfGCfUsUsU-3′ 3′-AsAsGAUCfUGfGAfCAfAAfACfGAfAfAfAsCfsA-5′

wherein the linker and the ligand are any linker described herein; the lowercase f represents that an adjacent nucleotide on the left side of the letter f is a 2′-fluoro modified nucleotide; the lowercase s represents that two adjacent nucleotides on the left and right sides of the letter s are linked by a phosphorothioate linkage; Ral is reverse abasic phosphoramidite described herein; and Tgn is Thymidine glycol nucleic acid.

In an optional embodiment, the compound has the structure shown below:

TABLE 9

| | Sense strand | Antisense strand |
|---|---|---|
| SNK- | 5′- | 5′- |
| 3464 | GsUsCAUCCfACfAfAfUGAGAGUA | UsGfsUACTgnCUCAUUGUfGGfAUGACs |
| | CA-A2- | GsA-3′(AGT) (see SEQ ID NO. 3 for |
| | UsCsUCCCAfCCfUfUfUUCUUCUA | unmodified sequence) |
| | AU-3′-TriGalNAc (see SEQ ID NO. 1 for | 5′- |
| | pre-A2 unmodified sequence, see | AsUfsUAGAfAGAAAAGGfUGfGGAGASC |
| | SEQ ID NO. 2 for post-A2 unmodified | SU-3′ (AGT) (see SEQ ID NO. 4 for |
| | sequence) | unmodified sequence) |
| | 5′-GsUsCAUCCfACfAfAfUGAGAGUACA-3′-A2-5′-UsCsUCCCAfCCfUfUfUUCUUCUAAU-3′-TriGalNAc | |
| | 3′- [symbol] -5′ | 3′- [symbol] -5′ |
| SNK- | 3′- | 5′- |
| 2960 | UsGsUUUUCGUUTUGfUCfCAGASU | AsCfsAAfAfAfGCfAAfAAfCAfGGfUCfUA |
| | SC-A2- | GsAsA-3′ (see SEQ ID NO. 7 for unmodified |
| | CsUsAGACCfUGfUTUUGCUUUUG | sequence) |
| | U-3′-L96 (see SEQ ID NO. 5 for pre- | |
| | A2 unmodified sequence, see SEQ ID | |
| | NO. 6 for post-A2 unmodified | |
| | sequence) | |
| | 3′-UsGsUUUUCGUUTUGfUCfCAGASUsC-5′-A2-5′-CsUsAGACCfUGfUTUUGCUUUUGU-3′-L96 | |
| | 5′-AsCfsAAfAfAfGCfAAfAAfCAfGGfUCfUAGsAsA-3′ | 3′- [symbol] -5′ |
| SNK- | 3′- | 5′- |
| 2998 | RalsUsAsUUUACAGAAfAfCfAAfCG | AsUfsAAAUfGUfCUUUGUfUGfCAAGCs |
| | UUsCfsG-A2- | UsUf-3′ (see SEQ ID NO. 10 for unmodified |
| | GsCfsUUGCAfACfAfAfAGACAUUU | sequence) |
| | AU-3′-TriGalNAc (see SEQ ID NO. 8 for | 5′- |
| | pre-A2 unmodified sequence, see | AsUfsAAAUfGUfCUUUGUfUGfCAAGCs |
| | SEQ ID NO. 9 for post-A2 unmodified | UsUf-3′ (see SEQ ID NO. 11 for unmodified |
| | sequence) | sequence) |
| | 3′-RalsUsAsUUUACAGAAfAfCfAAfCGUUsCfsG-5′-A2-5′- [symbol] -3′-TriGalNAc | |
| | 5′- [symbol] -3′ | 3′- [symbol] -5′ |
| SNK- | 3′- | 5′- |
| 3467 | UsAsUCUACAUCAfUfCfUUfAAAGs | AsUfsAGfAUfGUAGUAGAfAUfUUCUCs |
| | AsGs-A2- | UsG-3′ (C3) (see SEQ ID NO. 14 for |
| | AsAsGfCAfAGfAUfAfUfUUfUUAfU | unmodified sequence) |
| | AfAUA-3′-TriGalNAc (see SEQ ID | 5′- |
| | NO. 12 for pre-A2 unmodified | UsAfsUfUAfUAAfAAfAUAUfCUfUGfCU |
| | sequence, see SEQ ID NO. 13 for post- | UsUsU-3′ (C5) (see SEQ ID NO. 15 for |
| | A2 unmodified sequence) | unmodified sequence) |
| | 3′- [symbol] -5′-A2-5′- [symbol] -3′-TriGalNAc | |
| | 5′- [symbol] -3′ | 3′- [symbol] -5′ |
| SNK- | 3′- | 5′- |
| 3010 | UsGsUGAAAAAACfAfAfAGfUGUf | AfsCfsACUUUfUUfUGUUUfCAfCAAACs |
| | UsUsG-A2- | AsA-3′ (AGT) (see SEQ ID NO. 18 for |
| | CsUsAGACCfUGfUTUUGCUUUUG | unmodified sequence) |
| | U-3′-TriGalNac (see SEQ ID NO. 16 for | 5′- |
| | pre-A2 unmodified sequence, see | AsCfsAAfAfAfGCfAAfAAfCAfGGfUCfUA |
| | SEQ ID NO. 17 for post-A2 | GsAsA-3′ (PCSK9) (see SEQ ID NO. 19 for |
| | unmodified sequence) | unmodified sequence) |
| | 3′-UsGsUGAAAAAACfAfAfAGfUGUfUsUsG-5′-A2-5′-CsUsAGACCfUGfUTUUGCUUUUGU-3′-TriGalNAc | |
| | 5′- [symbol] -3′ | 3′- [symbol] -5′ |
| SNK- | 3′- | 5′- |
| 3005 | RalsUsUsUUUCUGACUfAfGfUUfUA | AsAfsAAAGfACUGAUCASAAfUAUGUSU |
| | UAsCsA-A2- | SG-3′ (ANGPTL3) (see SEQ ID NO. 22 for |
| | CsUsAGACCfUGfUTUUGCUUUUG | unmodified sequence) |
| | U-3′-TriGalNAc (see SEQ ID NO. 20 for | 5′- |
| | pre-A2 unmodified sequence, see | AsCfsAAfAfAfGCfAAfAAfCAfGGfUCfUA |
| | SEQ ID NO. 21 for post-A2 | GsAsA-3′ (PCSK9) (see SEQ ID NO. 23 for |
| | unmodified sequence) | unmodified sequence) |
| | 3′-RalsUsUsUUUCUGACUfAfGfUUfUAUAsCsA-5′-A2-5′-CsUsAGACCfUGfUTUUGCUUUUGU-3′-TriGalNAc | |
| | 5′- [symbol] -3′ | 3′- [symbol] -5′ |
| SNK- | 3′- | 5′- |
| 3461 | RalsUsUsUUUCUGACUfAfGfUUfUA | AsAfsAAAGfACUGAUCASAAfUAUGUSU |
| | UAsCsA-A4- | SG-3′ (ANGPTL3) (see SEQ ID NO. 26 for |
| | CsUsAGACCfUGfUTUUGCUUUUG | unmodified sequence) |
| | U-3′-TriGalNAc (see SEQ ID NO. 24 for | 5′- |
| | pre-A4 unmodified sequence, see | AsCfsAAfAfAfGCfAAfAAfCAfGGfUCfUA |
| | SEQ ID NO. 25 for post-A4 | GsAsA-3′ (PCSK9) (see SEQ ID NO. 27 for |
| | unmodified sequence) | unmodified sequence) |
| | 3′-RalsUsUsUUUCUGACUfAfGfUUfUAUAsCsA-5′-A4-5′-CsUsAGACCfUGfUTUUGCUUUUGU-3′-TriGalNAc | |
| | 5′- [symbol] -3′ | 3′- [symbol] -5′ |
| SNK- 3008 | 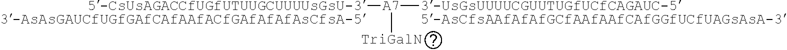 | |
| SNK- | 5′- | 5′- |
| 3468 | UsGfsACfUUfUGfAAfCACAfUGfUU | AfsGsCfAAfCAfUGfUfGfUUfCAfAAfGUfs |
| | fGCfUsUsU-A2- | CsAf -3′ (CFB) (see SEQ ID NO. 30 for |
| | CsUsAGACCfUGfUTUUGCUUUUG | unmodified sequence) |
| | U-3′-TriGalNAc (see SEQ ID NO. 28 for | 5′- |
| | pre-A2 unmodified sequence, see | AsCfsAAfAfAfGCfAAfAAfCAfGGfUCfUA |
| | SEQ ID NO. 29 for post-A2 | GsAsA-3′ (PCSK9) (see SEQ ID NO. 31 for |
| | unmodified sequence) | unmodified sequence) |
| | | [symbol] |
| | [symbol] -A2- [symbol] -TriGalNAc | |
| SNK- | 3′- | 5′- |
| 2192 | UAAUCUUCUUUfUfCfCAfCCCUsC | AsUfsUAGAfAGAAAAGGfUGfGGAGAsC |
| | SU-UsUUsU- | SU-3′ (AGT) (see SEQ ID NO. 34 for |
| | CsUsAGACCfUGfUTUUGCUUUUG | unmodified sequence) |
| | U-3′-TriGalNAc (see SEQ ID NO. 32 for | |
| | pre-UsUUsU unmodified sequence, | |
| | see SEQ ID NO. 33 for post-UsUUsU | |
| | unmodified sequence) | |
| | [symbol] 3′- [symbol] -5′-UsUUsU-5′- [symbol] -3′-TriGalNAc | |
| | [symbol] 5′- [symbol] -3′ | 3′- [symbol] -5′ |
| SNK- | 3′- | 5′- |
| 3466 | UAAUCUUCUUUfUfCfCAfCCCUsC | AsUfsUAGAfAGAAAAGGfUGGGAGASC |
| | SU-A2- | SU-3′ (AGT) (see SEQ ID NO. 37 for |
| | CsUsAGACCfUGfUTUUGCUUUUG | unmodified sequence) |
| | U-3′-TriGalNAc (see SEQ ID NO. 35 for | |
| | pre-A2 unmodified sequence, see | |
| | SEQ ID NO. 36 for post-A2 | |
| | unmodified sequence) | |
| | [symbol] 3′- [symbol] -5′-A2-5′- [symbol] -3′-TriGalNAc | |
| | [symbol] 5′- [symbol] -3′ | 3′- [symbol] -5′ |
| SNK- | 3′- | 5′- |
| 2959 | UsGsUUUUCGUUTUGfUCfCAGASU | AsCfsAAfAfAfGCfAAfAAfCAfGGfUCfUA |
| | sC-Linker- | GsAsA-3′ (see SEQ ID NO. 40 for |
| | CsUsAGACCfUGfUTUUGCUUUUG | unmodified sequence) |
| | U-3′-L96 (see SEQ ID NO. 38 for pre- | |
| | linker unmodified sequence, see SEQ | |
| | ID NO. 39 for post-linker unmodified | |
| | sequence) | |
| | 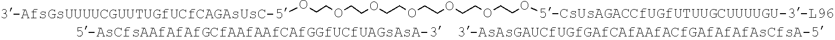 | |
| SNK- | 3′- | 5′- |
| 3469 | AfsCsUfGAfAAfCUfUGfUfGfUAfCA | UsGfsACfUUfUGfAAfCACAfUGfUUfGCf |
| | fACfsGsAf-A2- | UsUsU-3′ (CFB) (see SEQ ID NO. 43 for |
| | CsUsAGACCfUGfUTUUGCUUUUG | unmodified sequence) |
| | U-3′-TriGalNAc (see SEQ ID NO. 41 for | |
| | pre-A2 unmodified sequence, see | |
| | SEQ ID NO. 42 for post-A2 | |
| | unmodified sequence) | |
| | [symbol] 3′- [symbol] -5′-A2-5′- [symbol] -3′-TriGalNAc | |
| | [symbol] 5′- [symbol] -3′ | 3′- [symbol] -5′ |
| SNK- | 3′- | 5′- |
| 3470 | RalsAfsCsUfGAfAAfCUfUGfUfGfUA | UsGfsACfUUfUGfAAfCACAfUGfUUfGCf |
| | fCAfACfsGsAf-A2- | UsUsU -3′ (CFB) (see SEQ ID NO. 46 for |
| | CsUsAGACCfUGfUTUUGCUUUUG | unmodified sequence) |
| | U-3′-TriGalNAc (see SEQ ID NO. 44 for | |
| | pre-A2 unmodified sequence, see | |
| | SEQ ID NO. 45 for post-A2 | |
| | unmodified sequence) | |
| | CASAOBORY -3 | |
| | [symbol] 3′- [symbol] -5′-A2-5′- [symbol] -3′-TriGalNAc | |
| | [symbol] 5′- [symbol] -3′ | 3′- [symbol] -5′ |

[symbol]

indicates data missing or illegible when filed

TriGalNAc is a ligand shown by formula (XI);
a CAS number of L96 is 1159408-62-4;
wherein the lowercase f represents that an adjacent nucleotide on the left side of the letter f is a 2′-fluoro modified nucleotide; the lowercase s represents that two adjacent nucleotides on the left and right sides of the letter s are linked by a phosphorothioate linkage; A2 is a linker with No. A2; A4 is a linker with No. A4; A7 is a linker with No. A7; and Tgn is Thymidine glycol nucleic acid.

In a third aspect, the present disclosure further provides a method of preparing the compounds as mentioned above, comprising:
(a) reacting a compound represented by Formula 1 with a compound represented by Formula 2;
(b) reacting a compound represented by Formula 1 with a compound represented by Formula 3; or
(c) reacting a compound represented by Formula 8 with a compound represented by Formula 11;

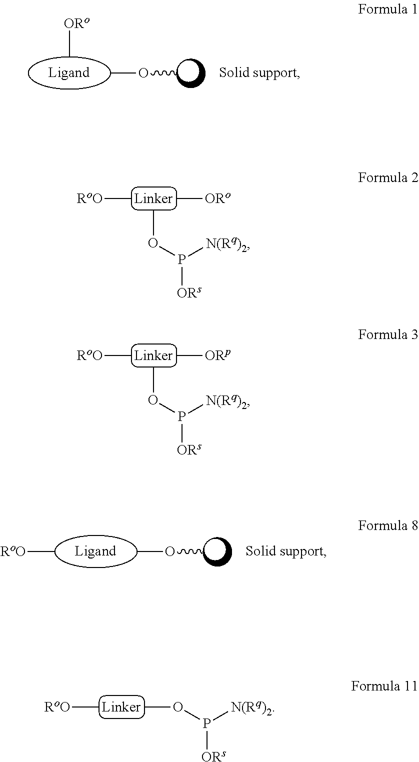

In an optional embodiment, the method further comprises:
reacting one or more of resulting intermediates with RNA phosphoramidites using a solid-phase phosphoramidite monomer chemical synthesis method.

In an optional embodiment, the method comprises:
(a) reacting the compound of Formula 1 with the compound of Formula 2 to produce an intermediate represented by Formula 4,

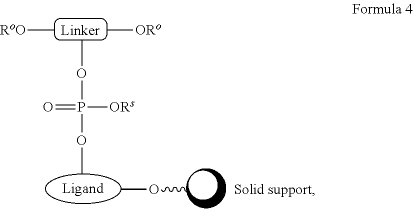

reacting the intermediate represented by Formula 4 with the RNA phosphoramidites to produce a compound represented by Formula 6,

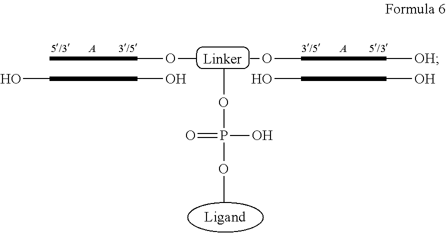

(b) reacting the compound of Formula 1 with the compound of Formula 3 to produce an intermediate represented by Formula 5,

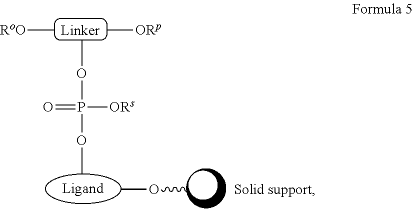

reacting the intermediate represented by Formula 5 with the RNA phosphoramidites to produce a compound represented by Formula 7,

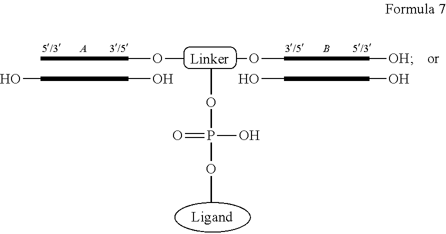

(c) reacting the compound represented by Formula 8 with the RNA phosphoramidites to produce an intermediate represented by Formula 10,

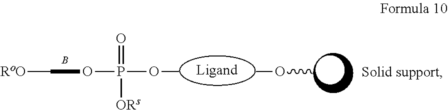

reacting the intermediate of Formula 10 with the compound of Formula 11 to produce an intermediate represented by Formula 12,

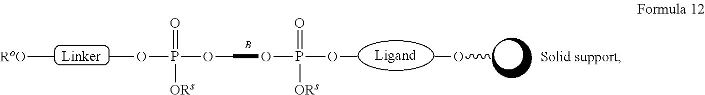

reacting the intermediate of Formula 12 with the RNA phosphoramidites to produce a compound represented by Formula 14,

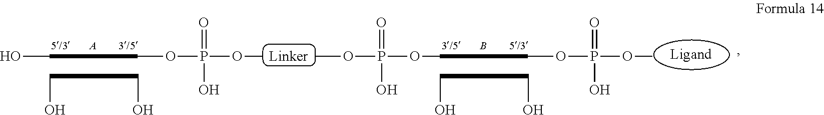

wherein A and B represent oligonucleotide fragments, R^{o} is a protecting group that is readily deprotected under acidic conditions, R^{p} is a protecting group that is readily deprotected under basic conditions, R^{q} is a C1-C3 alkyl group, R^{s} is a cyano C1-C3 alkyl group.

In an optional embodiment, the method R^{o} is 4,4′-di-C1-C3 alkyl oxytrityl, R^{p} is

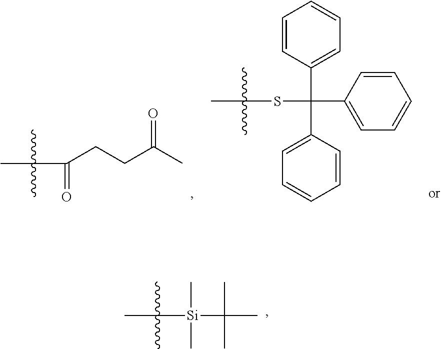

R^{q} is isopropyl, R^{s} is cyanoethyl.

In an optional embodiment, R^{o} is

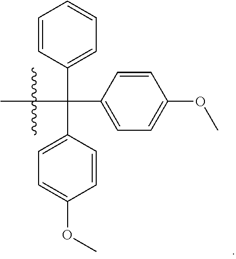 [SMILES: *C(c1ccccc1)(c1ccc(OC)cc1)c1ccc(OC)cc1]

In an optional embodiment, the compound including the linker, the ligand and the oligonucleotides of the present disclosure is synthesized using the method shown in FIG. 1 or FIG. 2. In FIG. 1 and FIG. 2, DMTO is

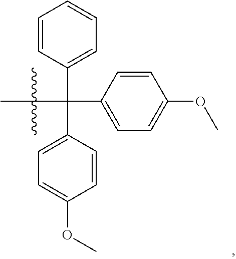 [SMILES: *C(c1ccccc1)(c1ccc(OC)cc1)c1ccc(OC)cc1]

R^{p} is a protecting group that is readily deprotected under basic conditions, preferably

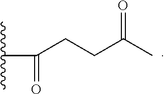 [SMILES: *C(=O)CCC(C)=O]

In a fourth aspect, the present disclosure provides a pharmaceutical composition comprising the compound as mentioned above and a pharmaceutically acceptable carrier.

In a fifth aspect, the present disclosure provides a method of delivering two or more oligonucleotides to a subject in need thereof, the method comprising administering the compound as mentioned above or the pharmaceutical composition as mentioned above. In an optional embodiment, the oligonucleotides are delivered to the same or a different position of the same target mRNA or are delivered to two or more target mRNAs.

In a sixth aspect, the present disclosure provides a method for inhibiting expression of a target gene in a subject in need thereof, comprising administering to the subject the compound as mentioned above or the pharmaceutical composition as mentioned above,
wherein the compound or the pharmaceutical composition inhibits expression of the target gene. The target gene includes but is not limited to C3, C5, ANGPTL3, CFB, AGT, AT3, PCSK9, KHK, INHBE, CNOTL6, GPR75, CDEB, XDH, LPA, APOB, APOC3, APP, MAPT, SOD1, APOE, PNPLA3, HSD17B13.

In a seventh aspect, the present disclosure further provides use of the linker described above in preparation of a reagent for delivering oligonucleotides.

In an eighth aspect, the present disclosure further provides use of the linker described above and the compound described above in preparation of drugs for inhibiting target genes.

In one aspect, the invention provides a compound comprising:
a linker comprising a structure represented by R₁-M′-R₂ (Formula A) as defined herein;
two or more oligonucleotides being conjugated to the linker; and
a ligand;
wherein the ligand delivers the two or more oligonucleotides to different positions of a target mRNA.

In one embodiment, the mode of binding between the compound and mRNA is shown in Scheme I or Scheme II:

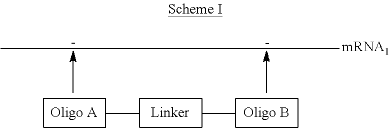

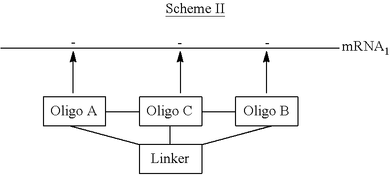

In another aspect, the invention provides a compound comprising:
a linker comprising a structure represented by R₁-M′-R₂ (Formula A) as defined herein;
two or more oligonucleotides being conjugated to the linker; and
a ligand;
wherein the ligand delivers the two or more oligonucleotides to the same position of a target mRNA.

In one embodiment, the mode of binding between the compound and mRNA is shown in Scheme III or Scheme IV:

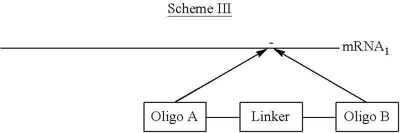

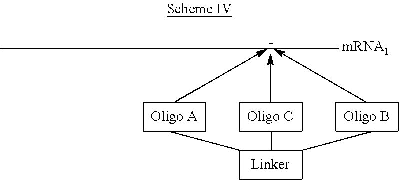

To evaluate the effects of different linkers on siRNA silencing activity, two identical siRNAs were linked by two different linkers on the 5′-end of sense strand and conjugated to a Tri-GalNAc for liver specific delivery (Table 10). The antisense strand was then duplexed with the sense strand.

TABLE 10

| | Sense strand | Antisense strand |
|---|---|---|
| Compound 1 | 3′-UsGsUUUUCGUUTUGfUCfCAGAsUsC-linker 1- | AsCfsAAfAfAfGCf |
| | CsUsAGACCfUGfUTUUGCUUUUGU-3′-Tri-GalNac | AAfAAfCAfGGfU |
| | (see SEQ ID NO. 5 for pre-A2 unmodified sequence, see | CfUAGsAsA (see |
| | SEQ ID NO. 6 for post-A2 unmodified sequence) | SEQ ID NO. 7 for |
| | | unmodified sequence) |
| Compound 2 | 3′-UsGsUUUUCGUUTUGfUCfCAGAsUsC-linker 2- | AsCfsAAfAfAfGCf |
| | CsUsAgACCfUGfUTUUGCUUUUGU-3′-Tri-GalNAc | AAfAAfCAfGGfU |
| | (see SEQ ID NO. 5 for pre-A2 unmodified sequence, see | CfUAGsAsA (see |
| | SEQ ID NO. 6 for post-A2 unmodified sequence) | SEQ ID NO. 7 for |
| | | unmodified sequence) |

Note:

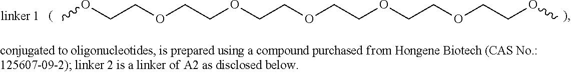

In another aspect, the invention provides a compound comprising:
a linker comprising a structure represented by R₁-M′-R₂ (Formula A) as defined herein;
two or more oligonucleotides being conjugated to the linker; and
a ligand;
wherein the ligand delivers the two or more oligonucleotides to two or more different target mRNAs.

In one embodiment, the mode of binding between the compound and mRNA is shown in Scheme V or Scheme VI:

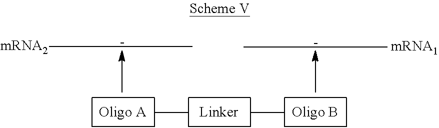

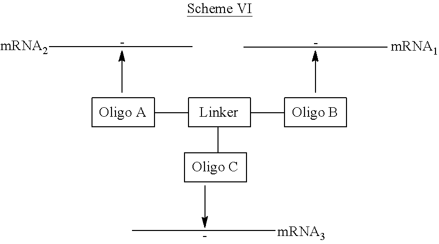

To confirm that compounds according to the invention targeting two target mRNAs can be achieved by linking through linker 2 (as described above), siRNAs targeting human C3 and human C5 were designed and linked on their sense strand as shown in Table 11.

TABLE 11

| | Sense strand | Antisense strand |
|---|---|---|
| Compound 3 | 3′-UsAsUCUACAUCAfUfCfUUfAAA | AsUfsAGfAUfGUAGUAGAfA |
| | GsAsGs-linker 2-sGsCsAAAC | UfUUCUCsUsG (C3) (see SEQ |
| | AfGCfAfAfUGCAAAACAC-3′-TriGalNac | ID NO. 14 for unmodified |
| | (see SEQ ID NO. 49 for pre-linker | sequence) |
| | unmodified sequence, | GfsUfsGfUUUUGCAUUGCfU |
| | see SEQ ID NO. 50 | GfUUUGCsUsU (C5) (see SEQ |
| | for post-linker unmodified sequence) | ID NO. 15 for unmodified |
| | | sequence) |

Linker 2 has the structure of A2 as disclosed herein.

Another compound (Compound 4) targeting human AGT and human PCSK9 was synthesized with linker 2 (Table 12).

TABLE 12

| | Sense strand | Antisense strands |
|---|---|---|
| Compound 4 | 3′-UsGsUGAAAAAACfAfAfAG | AfsCfsACUUUfUUfUGU |
| | fUGUfUsUsG-linker2- | UUfCAfCAAACsAsA (AGT) (see |
| | CsUsAGACCfUGfUTUU | SEQ ID NO. 18 for unmodified |
| | GCUUUUGU-3′-TriGalNac (see SEQ | sequence) |
| | ID NO. 16 for pre- | AsCfsAAfAfAfGCfAAfA |
| | linker unmodified | AfCAfGGfUCfUAGsAsA (PCSK9) |
| | sequence, see SEQ ID NO. 17 for | (see SEQ ID NO. 19 for unmodified |
| | post-linker unmodified sequence) | sequence) |

Linker 2 has the structure of A2 as disclosed herein.

As evidenced herein, the linking two or more siRNAs targeting the same or different gene targets (e.g., target mRNA) with a linker of Formula A is achievable and has superior results. The gene targets include but are not limited to C3, C5, ANGPTL3, CFB, AGT, AT3, PCSK9, KHK, INHBE, CNOTL6, GPR75, CDEB, XDH, LPA, APOB, APOC3, APP, MAPT, SOD1, APOE, PNPLA3, HSD17B13. In some embodiments, the two or more siRNAs are targeting the same gene target. In some embodiments, the two or more siRNAs are targeting different gene targets.

In any of the compounds according to any aspects of the present invention, the ligand conjugates to one or more of the two or more oligonucleotides or to the linker. Non-limiting examples are shown in Formulas VII-X.

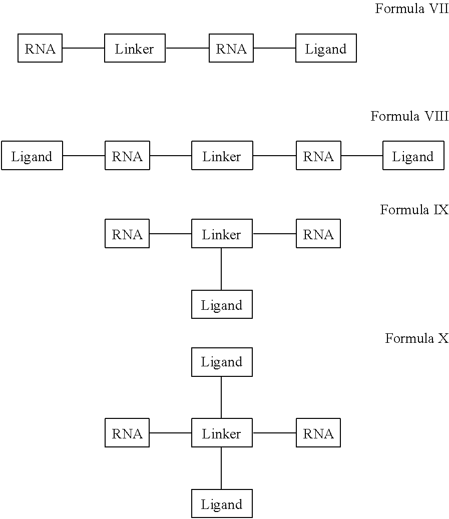

In some embodiments, the ligand is selected from lipid nanoparticles (LNPs, adeno-associated viruses (AAVs), Polymeric Nanoparticles, Liposomes, Polymeric Micelles, Inorganic Nanoparticles, Exosomes, and the like. In embodiments, the ligand encapsulates the linker and the two or more oligonucleotides.

The ligand is capable of delivering an RNA to a cell (e.g., a cell receptor or surface of a cell).

The ligand can be chosen from lipid, Galnac, fatty acid, cholesterol, polysaccharide, peptide, antibody, aptamer, nano-particle, and other small molecules may specifically bind the certain cell surface protein.

The ligand can be a compound T described in Chinese patent application CN202111499092.9A.

According to some embodiments of the present disclosure, the ligand comprises a structure shown by formula (XI) or formula (XII):

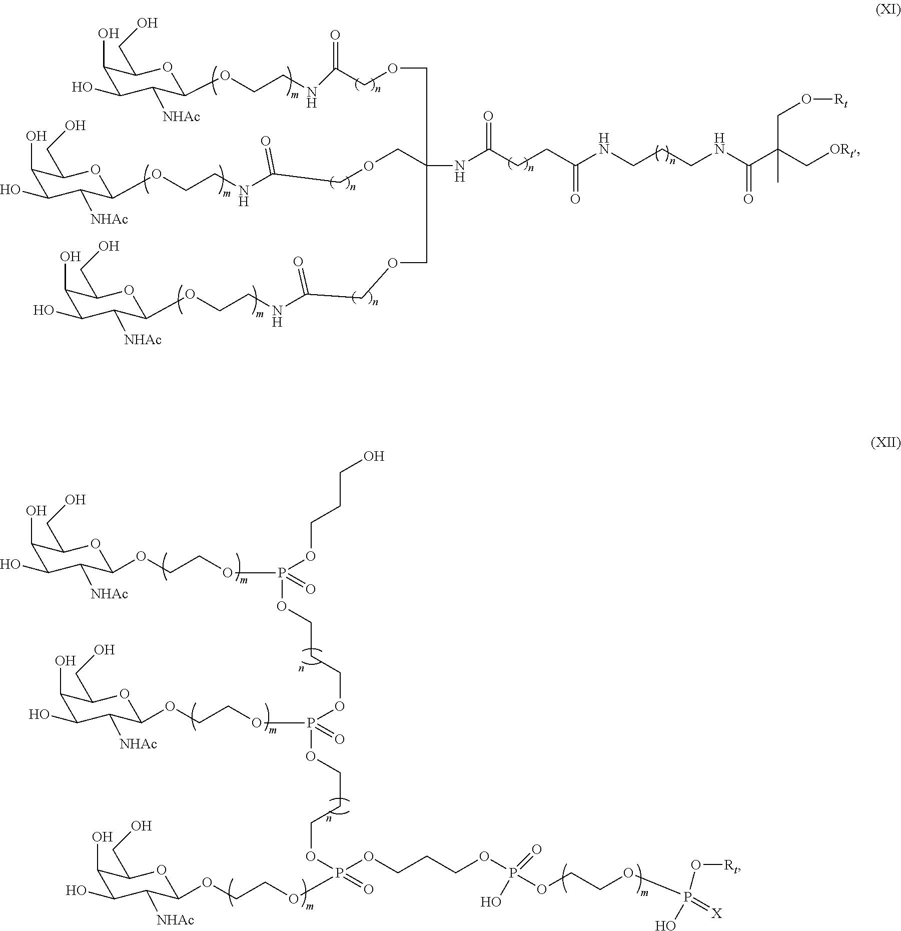

wherein m and n on formula (XI) or formula (XII) are each independently an integer of 0-6; Rₜ and R_{t′} are each independently selected from H, hydroxyl protecting group, a compound according to any aspect of the invention (“Nu”), solid phase carrier, C2-C6 carboxyl, carboxylate or amide group; wherein the hydroxyl protecting group is one selected from the group consisting of 4,4′-dimethoxytrityl, 4-methoxytrityl, trityl, t-butyldimethylsilyl, 4-oxopentanoyl, 2-cyanoethyl, 4-pentenoyl and acyloxyalkyl group. Preferably, Rₜ and R_{t′} are a compound according to any aspect of the invention (Nu) as described herein.

According to some embodiments of the present disclosure, the conjugate has a structure represented by formula (601), (602), (603), (604), (605), (606) or (607):

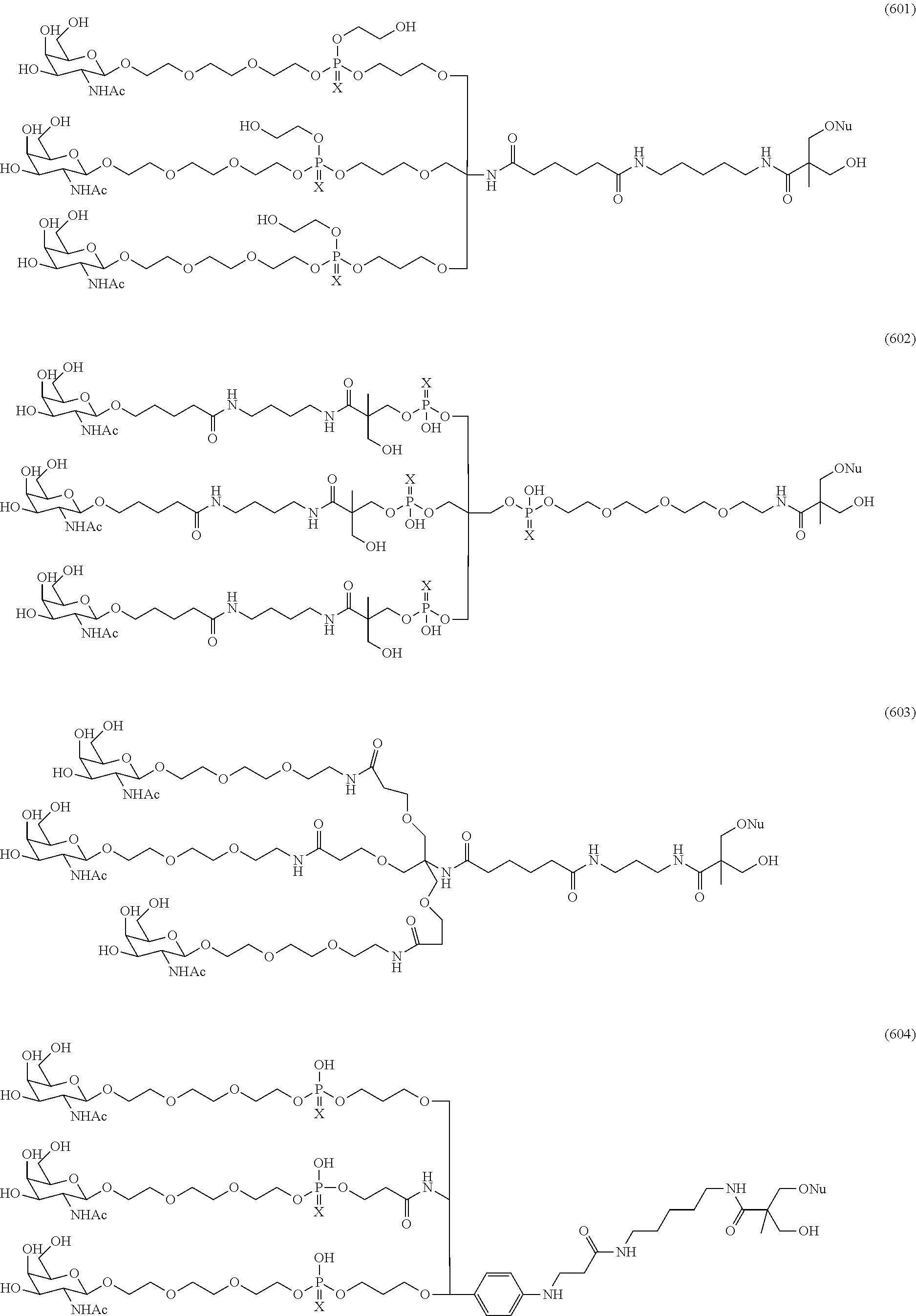

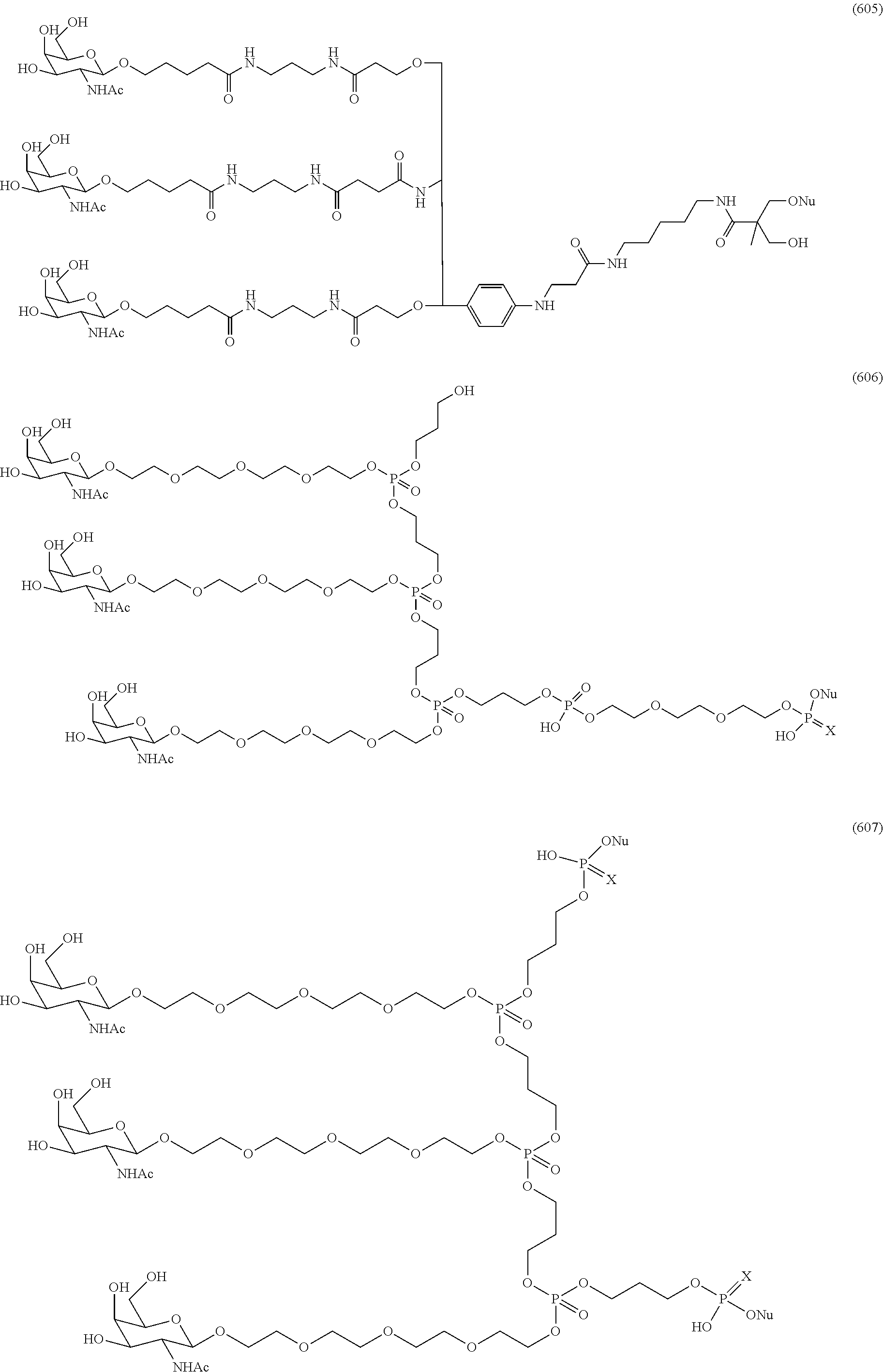

wherein X on formula (601), (602), (603), (604), (605), (606) or (607) is O or S; and Nu is a compound according to any aspect of the invention as described herein.

In the present disclosure, a conjugate with the structure shown by formula (603) may be obtained by contacting the compound shown by formula (603A) with an anhydride (e.g., succinic anhydride) to obtain the corresponding carboxylate (603B), the carboxylate (603B) is further connected to a solid phase to obtain a compound (603C), which is further attached on an active group Nu (Nu=a compound according to any aspect of the invention).

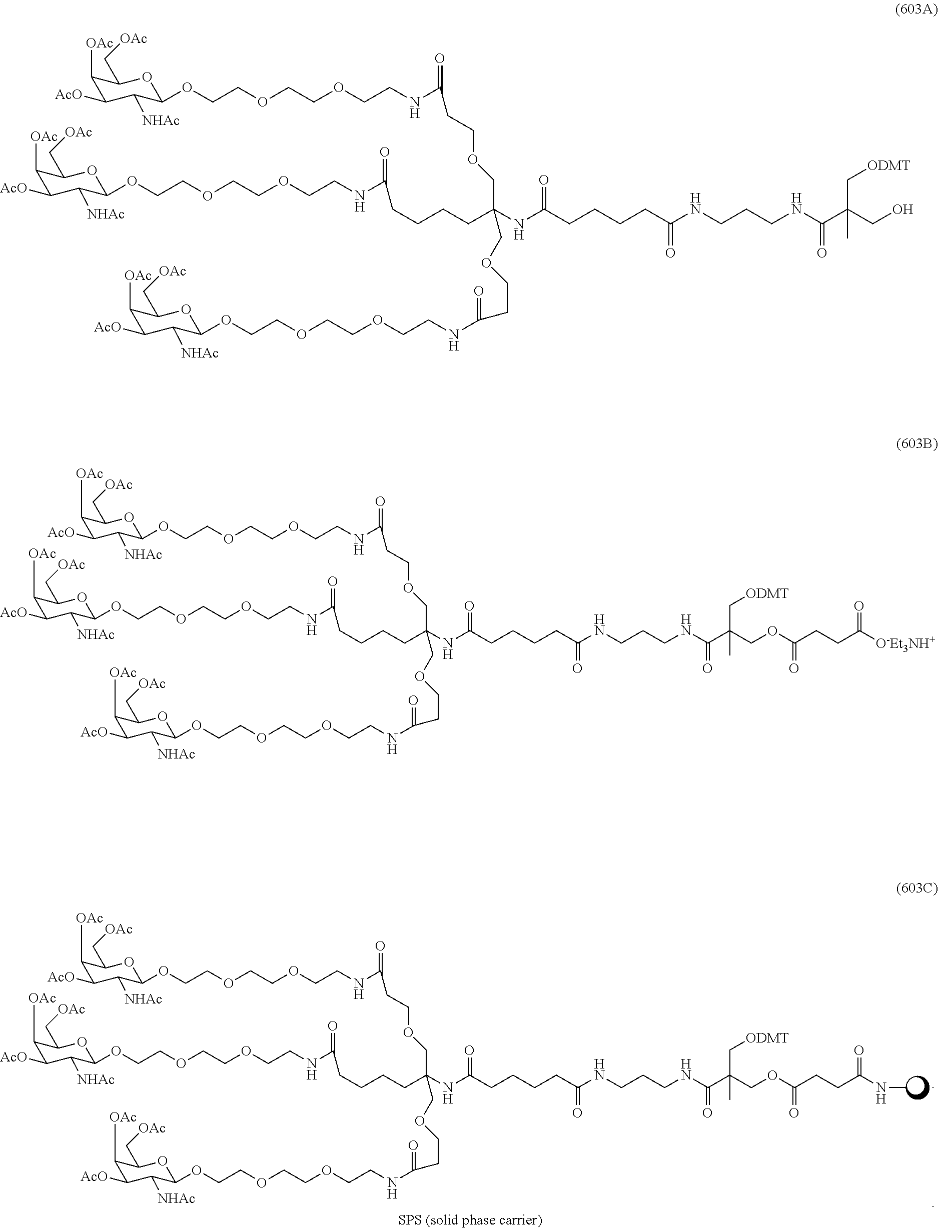

Preferably, the conjugated oligonucleotide comprises the structure:

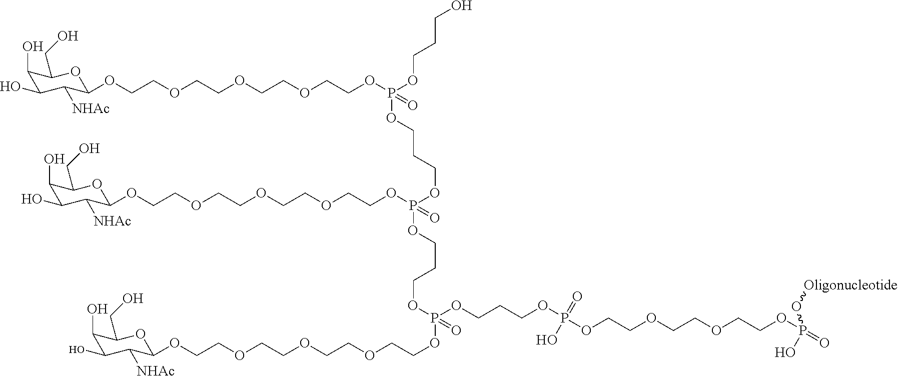

Preferably, the conjugated oligonucleotide comprises the structure:

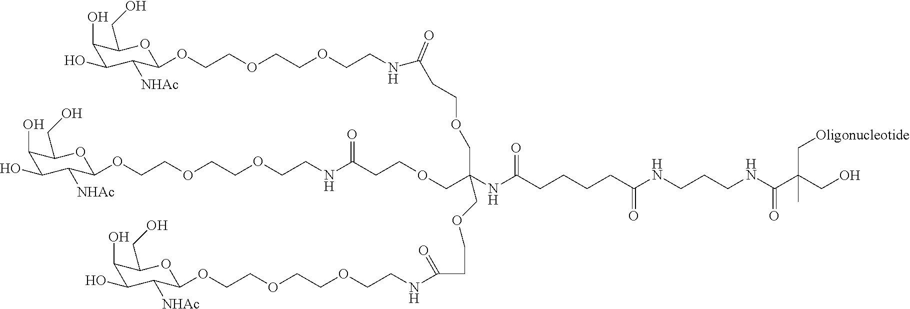

In some embodiments, where the two or more oligonucleotides are siRNA, the linker conjugates to a sense strand of one siRNA and an antisense strand of another siRNA (see e.g., Formula III). The siRNAs can be same or different.

In some embodiments, where the two or more oligonucleotides are siRNA, the linker conjugates to a sense strand of one siRNA and a sense strand of another siRNA (see e.g., Formula IV). The siRNAs can be same or different.

In some embodiments, where the two or more oligonucleotides are siRNA, the linker conjugates to an antisense strand of one siRNA and an antisense strand of another siRNA (see e.g., Formula V). The siRNAs can be same or different.

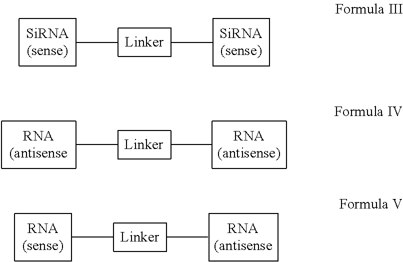

In some embodiments, where the two or more oligonucleotides are single-stranded RNA molecules, the linker conjugates to 3′ end of one RNA and 3′ end of the other RNA (see e.g., Formula VI). The RNA can be same or different.

In some embodiments, where the two or more oligonucleotides are single-stranded RNA molecules, the linker conjugates to 3′ end of an RNA and 5′ end of an RNA (see e.g., Formula VII). The RNA can be same or different.

In some embodiments, where the two or more oligonucleotides are single-stranded RNA molecules, the linker conjugates to 5′ end of an RNA and 5′ end of an RNA (see e.g., Formula VIII). The RNA can be same or different.

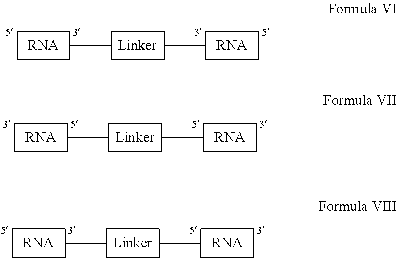

In some embodiments, wherein the two or more oligonucleotides are RNA molecules, and wherein the linker is conjugated to a sense strand of a first RNA (RNA₁) and to an antisense strand of a second RNA (RNA₂), the linker conjugates to 5′ end of the sense strand of the RNA₁ and 3′ end of the antisense strand of the RNA₂. (see e.g., Formula IX).

In some embodiments, wherein the two or more oligonucleotides are RNA molecules, and wherein the linker is conjugated to a sense strand of a first RNA (RNA₁) and to an antisense strand of a second RNA (RNA₂), the linker conjugates to 3′ end of the sense strand of the RNA₁ and 5′ end of the antisense strand of the RNA₂. (see e.g., Formula X).

In embodiments, the RNA₁ and the RNA₂ are siRNAs, wherein the RNA₁ and the RNA₂ are the same or different siRNAs.

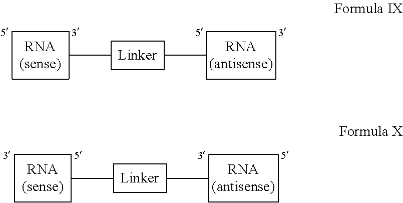

Without wishing to be bound to any particular theory, compounds according to any of the aspects of the present invention have one or more of the following benefits:
a) Enabling a single ligand conjugation delivery of multiple oligonucleotides overcomes receptor saturation, and improves oligonucleotide efficacy;
b) Using specific and proprietary linker and linked structure is able to optimize and enhance activity of each linked oligonucleotide;
c) Overall more favorable pharmacokinetics and pharmacodynamics comparing to conventional types of compound;
d) Linking the sense strand of an RNA to the antisense strand of a different RNA is able to achieve multiple target knock-down, or provide better knock-down of the same target;
e) Avoid hair-pin structure formation by linking the sense and the antisense strands of the same RNA;
f) Are of higher molecule weight in vivo for favorable pharmacokinetics;
g) 3′-5′ linkage facilitate synthesis using standard phosphoramidite chemistry;
h) Optimizes RNAi activities of all linked RNAs;
i) Without wishing to be bound to any particular theory, compounds according to any of the aspects of the present invention have a molecule weight greater than 20,000 have the following benefits:
j) Having higher molecular weight and size is able to avoid quick renal clearance comparing to regular compounds used for delivering a targeted drug;
k) More interaction with plasma proteins; and
l) More durable activities in vivo because of slower release from endosome.

In any of the aspects of the present invention, the two or more oligonucleotides independently comprise a single or double stranded natural or modified DNA or RNA (the natural or modified DNA or RNA can be partial or full stranded), aptamer, CpG oligonucleotide, small circular RNA, small circular DNA, small hairpin RNA, short hairpin RNA, short interfering-RNA (siRNA), microRNA (miRNA), DNA-directed-RNAi (ddRNAi), and single stranded RNAi (ssERNAi)), or small activation RNA (saRNA).

Modified DNA and RNA typically involve changes to the nucleotide bases, sugar molecules (ribose or deoxyribose), heterocyclic, or phosphate groups. Modified DNA or RNA include but are not limited to 2′-OMe, 2′-F, PNA, morpholino, 2′-MOE, LNA, cEt, and the like. In addition, the DNA or RNA backbone can include but are not limited to internucleotide linkages selected from phosphorothioate, phosphodiester, phosphorodiamidate morpholino, methyl phosphonate, methyl phosphotriester, phosphoramidate, phosphorodithioate, or any combination of thereof. The modified DNA and RNA can also be modified to have a modified 5′-phosphate group such as 5′-(E) VP.

Preferably at least one of the two or more oligonucleotides is a short interfering-RNA (siRNA).

The two or more oligonucleotides can be linear or circular.

The two or more oligonucleotides can be same or different.

The linker according to the present invention comprises a structure represented by Formula A:
R₁-M′-R₂ (Formula A)
wherein:
M′ is —(CH₂)—, —(CHQ″)—, —NQ″-, —OP(═O)(X)O—,

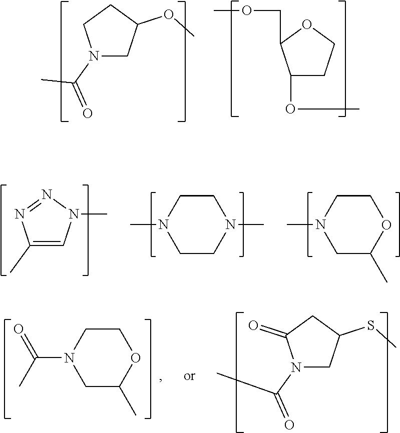

Q″ is —Cl, —Br, —I, —CH3, —CH(CH3)2, —OH, —CH2CH2OH, —CH2CH2CH2OH, —CO2H, —COCH3, —CONH—, CH2CONH—CH2CH2CO2H, —CH2CH2CH2CO2H, —CH2CH2NH2, —CH2CH2CH2NH2, —CONHCH3, —CH2CH2CONHCH3, —CH2CH2CH2CONHCH3, —O(CH2)k′-, —O—CH2-CH2-CH2-, —O—CH2-CH2-CH2-CH2-, —O—CH2-CH2-CH2-CH2-CH2-, —OPO3H—, —O—PSO2H—, —C(O)(CH2)k′-, —CH2-NH—CO—, —CH2-CH2-NH—CO—, —CH2-CH2-CH2-NH—CO—, —OP(═O)OH2, —OP(═S)OH2, —OP(═O)(CH3)OH, —OP(═O)(CH2CH3)OH, —OP(═O)(OCH3)OH, —OP(═O)(NH2)OH, —OP(═O)(OCH2CH3)OH, —OP(═O)(NHCH3)OH, —OP(═O)(NHCH2CH3)OH, —OP(═S)(CH3)OH, —OP(═S)CH2CH3)OH, —OP(═S)(OCH3)OH, —OP(═S)(OCH2CH3)OH, —OP(═S)(NH2)OH, —OP(═S)(NHCH3)OH, —OP(═S)(NHCH2CH3)OH, —OP(═S)SHOH, —OS(═O)2OH, or OS(═O)OH, —OP(═O)OH—R3, —OP(═S)OH—R3, —OP(═O)(CH3)-R3, —OP(═O)(CH2CH3)-R3, —OP(═O)(OCH3)-R3, —OP(═O)(NH2)-R3, —(CH2)n″-, —(OCH2CH2)n″-O—, —(CH═CH—CH2)n″-, —(C6H4—CH2—)n″-, —CO—, —(C6H10)n″-, or —(C6H10-CH2—)n″-;
R1 or R2 are independently selected from —NHCO—, —S—S—, —OCH2CH2O—, —OCH2CH2S—SCH2CH2O—, —NHCH2CH2OCH2CH2NH—, —NH—(CH2)n″-NH—, —O—CH2-CH2-CH2-, —O—CH2-CH2-CH2-CH2-, —O—CH2-CH2-CH2-CH2-CH2-, —OPO3H—, —O—PSO2H—, —(CH2)k′NH—CO(CH2)j′-, —CH2-NH—CO—, —CH2-CH2-NH—CO—, —CH2-CH2-CH2-NH—CO—, —OP(═O)OHO—, —OP(═S)OHO—, —OP(═O)(CH3)O—, —OP(═O)(CH2CH3)O—, —OP(═O)(OCH3)O—, —OP(═O)(OCH2CH3)O—, —OP(═O)(NH2)O—, —OP(═O)(NHCH3)O—, —OP(═O)(NHCH2CH3)O—, —OP(═S)(CH3)O—, —OP(═S)CH2CH3)O—, —OP(═S)(OCH3)O—, —OP(═S)(OCH2CH3)O—, —OP(═S)(NH2)O—, —OP(═S)(NHCH3)O—, —OP(═S)(NHCH2CH3)O—, —OP(═S)SHO—, —OS(═O)2O—, OS(═O)O—, —OP(═O)OH—R3, —OP(═S)OH—R3, —OP(═O)(CH3)-R3, —OP(═O)(CH2CH3)-R3, —OP(═O)(OCH3)-R3, —OP(═O)(NH2)-R3, —(CH2)n″-, —(OCH2CH2)n″-O—, —(CH═CH—CH2)n″-, —(C6H4—CH2—)n″-, —CO—, —(C6H10)n″-, —(C6H10-CH2—)n″-;
n″ is 1 to 50; k′ is 1 to 10, preferably 1-5; j′ is 1 to 10, preferably 1-5;
X is O or S atom, or —CH₃, OCH₃, —NH₂; and
R₃ is an oligonucleotide or a delivery conjugate.

In embodiments, the linker according to the present invention comprises a structure represented by Formula A′:

R₁-M″-R₂ (Formula A′)
wherein:
M″ is —(CH₂)—, —(CHQ′″)—, —NQ′″-, —OP(═O)(X)O—, Q′″ is —OH, —O(CH₂)k″- —C(O)(CH₂)k″-,
R₁ or R₂ are independently selected from —(CH₂)k″NHCO(CH₂)j″-, —NHCO—, —NHCH2CH₂OCH₂CH₂NH—, —NH—(CH₂)n′″-NH—;
n″′ is 1 to 50
k″ is 1 to 10, preferably 1-5;
j″ is 1 to 10, preferably 1-5.

In embodiments, M′, M″, Q″, Q′″, R1, and R2 independently may be further substituted.

R1 and R² may be the same or different. In embodiments, R¹ and R² are the same. In embodiments, R¹ and R² are different.

Commercial linkers generally contain a saturated alkyl chain or polyethylene glycol chain. In contrast, linkers of Formula A comprise a symmetrical di-peptide bond which may improve oligonucleotide conjugates in water solubility or cell penetration. It may also improve the biological activity in the field of pharmacology.

In embodiments according to any aspect of the present invention, wherein the two or more oligonucleotides are different siRNAs, and R¹ or R₂ are independently selected from —(CH2)k″NHCO(CH2)j″-, —NHCO—, —NHCH2CH2OCH2CH2NH—, or —NH—(CH2)n′″-NH—.

In embodiments according to any aspect of the present invention, wherein the two or more oligonucleotides are different siRNAs, and R1 or R2 are —(CH₂)k″NHCO(CH₂)j″-.

When R1 and R2 are chosen from the aforementioned group, the claimed compound demonstrates promising biological activities and effectively sustains gene silencing in a persistent manner. Additionally, this structure simplifies the compound synthesis process.

Traditionally, when the two or more oligonucleotides (e.g., oligonucleotide A and oligonucleotide B) are distinct siRNAs, the synthesis process of a compound typically necessitates the use of at least two deprotection strategies on the linker. However, Linkers of Formula A allow for the synthesis of the claimed compound using only one type of deprotection reagent. Consequently, the reduction in byproducts during the process leads to a significant enhancement in the quality of the final product.

In embodiments according to any aspect of the present invention, the linker is selected from Formula A2, A7, A23, A26, A″2, A″4, A″5.

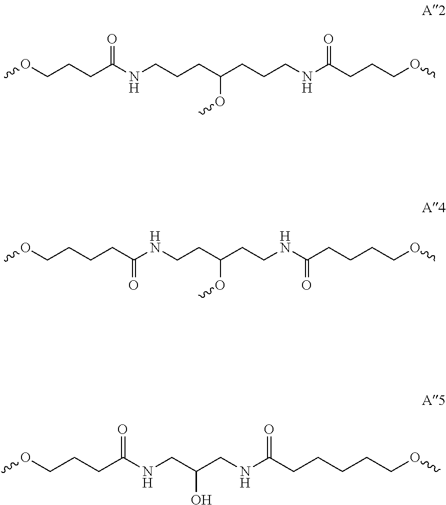

Wherein the terminal “O” at R1 and R2 are the binding point on the oligonucleotide for conjugation to the linker.

In embodiments, phosphoric bonds such as but not limited to phosphodiester, phosphorothioate, phosphonate, are phosphoramidate, bond the linkers and oligonucleotides.

The technical solutions of the present disclosure have the following advantages:
1. The linker provided by the present disclosure may be hydrolyzed to release the oligonucleotides linked thereto, and the oligonucleotides bind to the same or different positions of the same target mRNA, or the oligonucleotides bind to two or more target mRNAs. The linker of the present disclosure may achieve delivery of two or more oligonucleotides through single administration, which greatly improves administration efficiency and lowers related costs.
2. The linker having the structure similar to that of A2 provided by the present disclosure achieves the better technical effects than a linker in the prior art.
3. The compound having the oligonucleotides modified by Ral provided by the present disclosure achieves the better effects, and achieves the better effects especially for CFB. The linkers conjugating to 3′ end of an RNA and 5′ end of an RNA (see e.g., Formula VII) show better stabilities especially in vivo.
4. The preparation method provided by the present disclosure achieves the better yield via fewer steps, and lowers the production cost.

### BRIEF DESCRIPTION OF THE DRAWINGS

In order to describe the technical solutions in detailed description of the present disclosure or in the prior art more clearly, the accompanying drawings that need to be used in the descriptions for the detailed description or the prior art will be briefly introduced below. Apparently, the accompanying drawings in the following descriptions are some embodiments of the present disclosure, and for those of ordinary skill in the art, other accompanying drawings may also be obtained from these accompanying drawings on the premise of no creative labor.

FIG. 1 shows a method for preparing a synthesized compound including a linker, a ligand and oligonucleotides of the present disclosure.

FIG. 2 shows another method for preparing a synthesized compound including a linker, a ligand and oligonucleotides of the present disclosure.

FIG. 3 shows a method for preparing oligonucleotides of the present disclosure.

FIG. 4 shows a result of Experimental example 1.

FIG. 5 shows a result of Experimental example 2.

FIG. 6 shows Plasma FXI levels in cynomolgus monkey.

FIG. 7 shows a result of Experimental example 4.

FIG. 8 shows a result of Experimental example 5.

FIG. 9 shows a result of Experimental example 6.

FIG. 10 shows a result of Experimental example 7.

FIG. 11 shows a result of Experimental example 8.

FIG. 12 shows a result of Experimental example 9. In FIG. 12, A shows a result for CFB, and B shows a result for PCSK9.

FIG. 13 shows a result of Experimental example 10. In FIG. 13, A shows a result for PCSK9, and B shows a result for AGT.

FIG. 14 shows a result of Experimental example 11.

FIG. 15 shows a result of Experimental example 12. In FIG. 15, A shows a result for CFB, and B shows a result for PCSK9.

### DETAILED DESCRIPTION

The following examples are provided for a better and further understanding of the present disclosure, are not limited to the optimal embodiments, and do not limit the content and scope of protection of the present disclosure, and any product that is the same as or similar to the present disclosure obtained by anyone under the inspiration of the present disclosure or by combining the present disclosure with other features in the prior art falls within the scope of protection of the present disclosure.

If specific experimental steps or conditions are not specified in the examples, the operation or conditions of conventional experimental steps described in the literature in this art can be carried out. Adopted reagents or instruments which are not specified with manufacturers are conventional commercially-available reagent products.

### Example 1 Synthesis and Preparation of Intermediate A2′ Including Linker A2

Preparation was conducted following a method below:

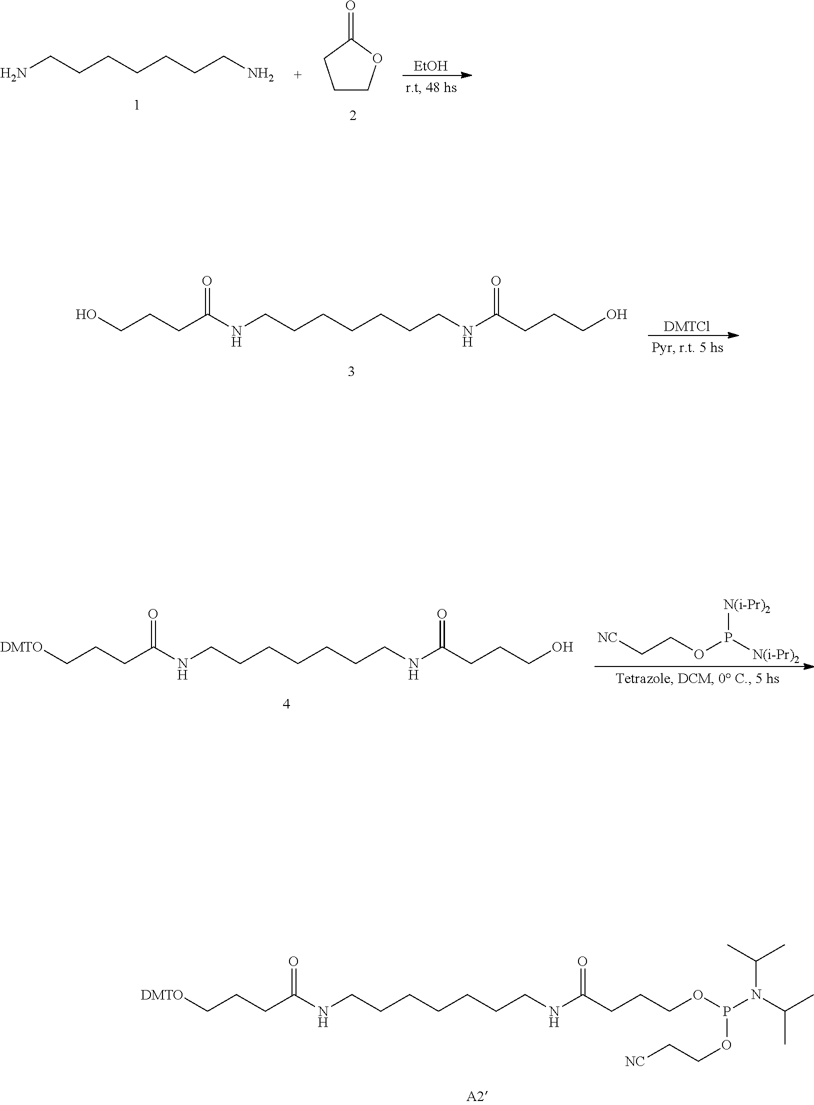

### Preparation of Intermediate 3

1,7-diaminoheptane 1 (2.2 g, 16.9 mmol) was dissolved in 50 mL of anhydrous ethanol at room temperature, and was fully dissolved. 1,4-butyrolactone 2 (3.2 mL, 54.08 mmol) was dropwise added into a reaction flask at room temperature. Stirring was continued at room temperature for 48 hours, then a reaction solution was poured into 250 mL of a saturated saline solution, 2×100 mL of ethyl acetate was added for extraction, the ethyl acetate was removed under reduced pressure, after being completely precipitated, a solid was filtered out, and flushed with ethyl acetate, and the ethyl acetate was removed under reduced pressure. Finally, a crude intermediate 3 (3.8 g, 75%) was obtained, and dried under vacuum at room temperature to be directly used for the next step of reaction.

### Preparation of Intermediate 4

4,4′-dimethoxytrityl chlorine (4.1 g, 12.1 mmol) was dissolved in 30 mL of anhydrous pyridine, and at room temperature, the compound 3 (3.72 g, 12.32 mmol) was dissolved in an anhydrous pyridine (10 mL) solution. The pyridine solution containing the 4,4′-dimethoxytrityl chlorine was slowly added into the pyridine solution containing the compound 3. This solution was continuously stirred at room temperature for 5 hours. 50 mL of water was added into a reaction mixture, and the reaction mixture was extracted with 2×100 mL of ethyl acetate. An organic phase was concentrated to be semi-dry after rotary evaporation, and continued to be purified by a silica gel chromatographic column, gradient elution was carried out once, an n-hexane solvent was used for washing first, then (n-hexane/ethyl acetate, 1:1, v/v) was used for elution, ethyl acetate continued to be used for elution, finally (ethyl acetate/methanol, 10:1, v/v) was used for elution, product components were collected, and the solvent was removed under reduced pressure to obtain a yellow solid intermediate 4 (2.9 g, 39.8%). The intermediate 4 was directly used for the next step of reaction.

### Preparation of Intermediate A2′

The intermediate 4 (2.8 g, 4.64 mmol) was dissolved in 40 mL of dry dichloromethane, and then 2-cyanoethyl N,N,N′,N′-tetraisopropyl-phosphordiamidite (1.67 mL, 5.57 mmol) was quickly added into the solution above. A reaction solution was stirred for 10 minutes in an ice bath under the protection of nitrogen. 9.3 mL of a 0.45 M tetrazolyl dichloromethane solution (4.18 mmol) was added into the reaction solution above, and the reaction solution continued to be stirred at room temperature under the protection of nitrogen to react for 5 hours. A reaction solution was poured into 100 mL of a saturated sodium bicarbonate solution, and extracted with 2×150 mL of ethyl acetate, an organic phase was separated out, anhydrous sodium sulfate was added for drying, and reduced pressure distillation was conducted to achieve a semi-dry state. Purification for separation was continued using a silica gel chromatographic column, gradient elution was carried out once, an n-hexane solvent was used for washing first, then (n-hexane/ethyl acetate, 2:1, v/v, 1% triethylamine) were used for elution, (n-hexane/ethyl acetate 1:1, v/v, 1% triethylamine) continued to be used for elution, finally (ethyl acetate, 1% triethylamine) were used for elution, product components were collected, and a solvent was removed under reduced pressure to obtain a white solid intermediate A2′ (2.8 g, 75%). ¹H NMR (CDCl₃): δ, 7.42-7.40 (m, 2H, trityl), 7.31-7.27 (m, 6H, trityl), 7.26-7.20 (m, 1H, trityl), 6.83-6.80 (m, 4H, trityl), 4.12-4.11 (m, 1H), 3.86-3.80 (m, 1H), 3.79 (s, 6H), 3.63-3.60 (m, 1H), 3.59-3.57 (m, 5H), 3.23-3.19 (m, 2H), 3.16-3.09 (m, 4H), 2.30-2.27 (m, 4H), 1.95-1.91 (m 4H), 1.30-1.27 (m, 12H), 1.25-1.18 (m, 12H) ppm. ³¹P NMR (CDCl₃): δ, 147.54 ppm.

### Example 2 Synthesis of Intermediate A4′ Including Linker A4, with Steps as Follows

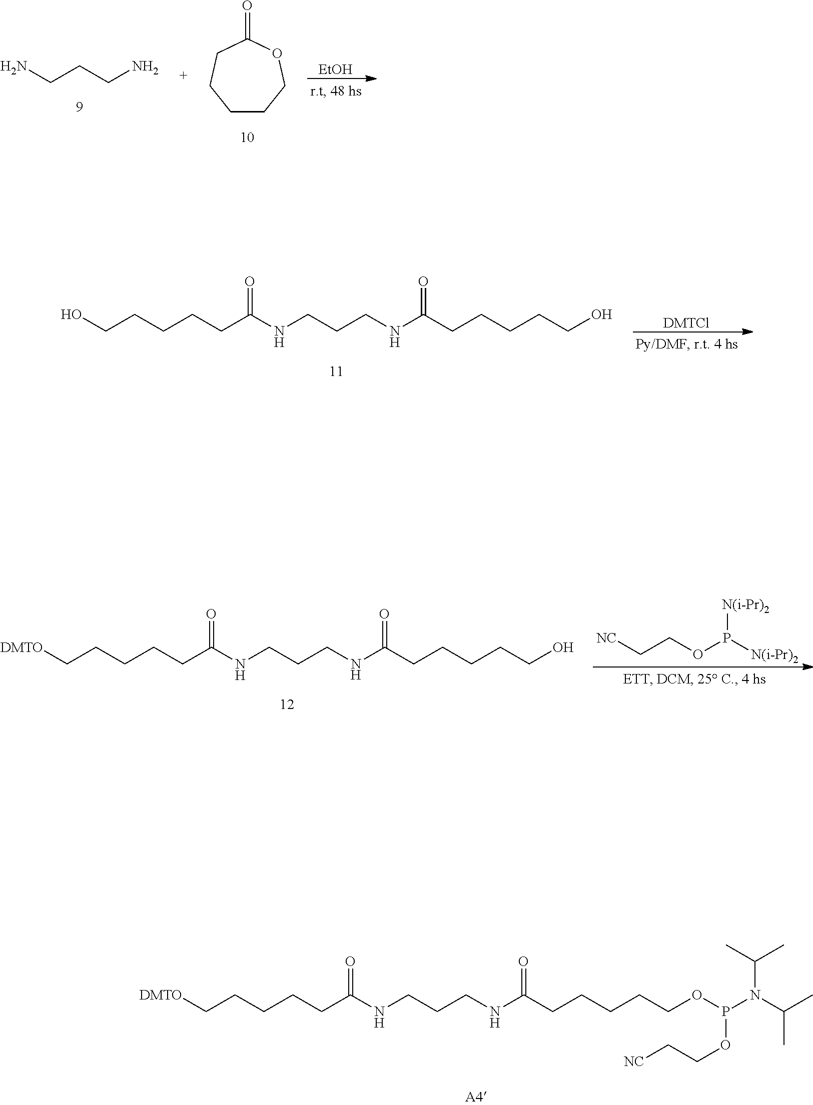

### Preparation of intermediate 11

1,3-diaminopropane 9 (8 mL, 96 mmol) was dissolved in 100 mL of anhydrous ethanol at room temperature until being completely dissolved. Then a solution of 6-caprolactone 10 (30 g, 0.263 mmol) in 50 mL of anhydrous ethanol was dropwise added to a reaction flask at room temperature. A mixture was stirred at room temperature for 48 hours, then poured into 500 mL of a saturated saline solution, and then extracted with 2×250 mL of ethyl acetate. The ethyl acetate was removed under reduced pressure, after being completely precipitated, a solid was filtered and washed with ethyl acetate, and then the ethyl acetate was removed under reduced pressure. Finally, a crude intermediate 11 (8.6 g, 29.6%) was dried under vacuum at room temperature to be directly used for the next step of reaction.

### Preparation of Intermediate 12

4,4′-dimethoxytrityl chlorine (9 g, 26.56 mmol) was dissolved in a mixed solvent including 50 mL of anhydrous pyridine and 25 mL of dimethylformamide. At room temperature, the intermediate 11 (8.6 g, 28.48 mmol) was dissolved in 50 mL of an anhydrous pyridine solution. The pyridine solution containing the 4,4′-dimethoxytrityl chlorine was slowly added into the pyridine solution containing the intermediate 11. The solution was stirred at room temperature for 4 hours. 100 ml of water was added into a reaction mixture, and then the reaction mixture was extracted with 2×250 mL of ethyl acetate. An organic phase was dried using anhydrous Na₂SO₄, then concentrated under reduced pressure to be semi-dry, further purified using silica gel column chromatography, and subjected to gradient elution. N-hexane was used to wash a column first, then (n-hexane/ethyl acetate, 1:1, v/v) was used for elution, then ethyl acetate was used for elution, and finally (ethyl acetate/methanol, 10:1, v/v, 1% triethanolamine) were used for elution. Product fractions were collected, a solvent was removed under reduced pressure, and a faint-yellow oil intermediate 12 (6.3 g, 36.6%) was obtained. The intermediate 12 was directly used for the next step of reaction.

### Preparation of Intermediate A4′

The intermediate 12 (6.2 g, 10.26 mmol) was dissolved in 50 mL of dry dichloromethane, and then 2-cyanoethyl N,N,N′,N′-tetraisopropyl-phosphordiamidite (3.4 g, 11.28 mmol) and 20 mL of dichloromethane were quickly added into the solution. A reaction mixture was stirred for 10 minutes in an ice bath under a nitrogen environment. Then, ethylthiotetrazole in 10 mL of dichloromethane (1.34 g, 10.27 mmol) was added into the reaction mixture, and the reaction mixture continued to be stirred at room temperature under the protection of nitrogen to react for 4 hours. The reaction mixture was poured into 150 mL of a saturated sodium bicarbonate solution, and extracted with 2× 250 mL of ethyl acetate. An organic phase was separated, dried using anhydrous sodium sulfate, and concentrated under reduced pressure to be semi-dry. Further purification and separation were conducted using silica gel column chromatography, and gradient elution was used. N-hexane was used to wash a column first, then (n-hexane/ethyl acetate, 2:1, v/v, 1% triethylamine) were used for elution, then (n-hexane/ethyl acetate, 1:1, v/v, 1% triethylamine) were used for elution, and finally (ethyl acetate, 1% triethylamine) were used for elution. Product fractions were collected, a solvent was removed under reduced pressure, and a white solid intermediate A4′ (6.8 g, 82%) was obtained. ¹H NMR (CDCl₃): d, 7.42-7.40 (m, 2H, triethyl), 7.31-7.25 (m, 6H, triethyl), 7.20-7.18 (m, 1H, triethyl), 6.82-6.80 (m, 4H, triethyl), 4.12-4.10 (m, 1H), 3.84-3.79 (m, 1H), 3.79 (s, 6H), 3.61-3.59 (m, 1H), 3.59-3.56 (m, 4H), 3.25-3.22 (m, 4H), 3.05-3.02 (m, 2H), 2.64-2.61 (m, 2H), 2.20-2.15 (m, 4H), 1.67-1.56 (m 10H), 1.41-1.38 (m, 4H), 1.28-1.24 (m, 3H), 1.18-1.16 (m, 12H) ppm. ³¹P NMR (CDCl₃): d, 147.38, 147.26 ppm.

### Example 3 Synthesis and Preparation of Intermediate A7′ Including Linker A7

### Synthesis of Intermediate A7′

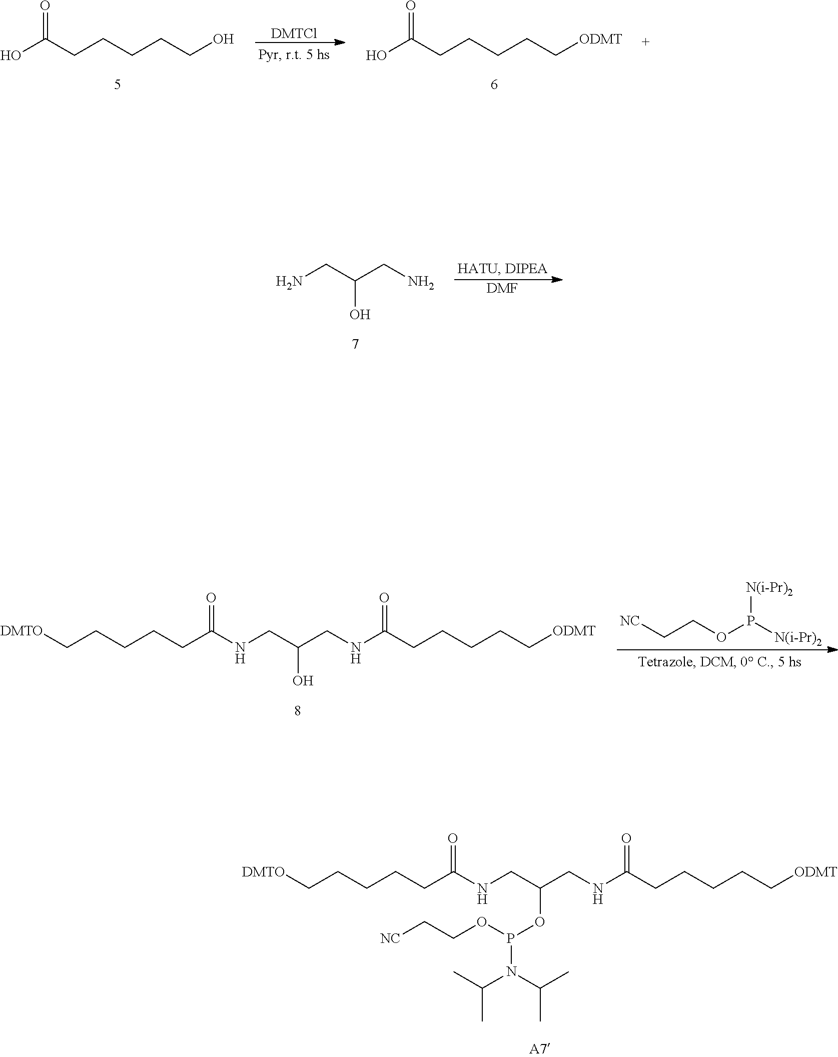

### Preparation of Intermediate 6

4,4′-dimethoxytrityl chlorine (7.44 g, 22 mmol) was dissolved in 10 mL of dichloromethane, and at room temperature, this solution was slowly dropwise added into an anhydrous pyridine (20 mL) solution containing 6-hydroxy-n-hexylic acid 5 (2.65 g, 20 mmol). This solution was continuously stirred at room temperature for 12 hours. 20 ml of water was added into a reaction mixture, and the reaction mixture was extracted with 2×100 mL of ethyl acetate. An organic phase was concentrated to be semi-dry after rotary evaporation, and a solvent continued to be removed under reduced pressure to obtain a yellow solid intermediate 6 (8.25 g, 95%). The product 6 was directly used for the next step of reaction.

### Preparation of Intermediate 8

The intermediate 6 (8.25 g, 19.8 mmol) was dissolved at room temperature in 50 mL of a mixed solvent (dried dimethylformamide and dried dichloromethane, v/v, 1:1). After full dissolution, 2-(7-azabenzotriazol-1-yl)-N,N,N′,N′-tetramethyluronium hexafluorophosphate (7.6 g, 20 mmol) and N,N-diisopropylethylamine (3.5 mL, 20 mmol) were gradually added into the resulting solution in an ice bath, and a reaction solution was fully stirred at 0° C. for 10 minutes. After an intermediate product was generated, a compound 1,3-diamine-2-propanol 7 (0.82 g, 9 mmol) was dissolved in 5 mL of dichloromethane, and then a solution was dropwise added into a reaction flask. A reaction solution was heated to a room temperature (25° C.) and continued to be stirred at the room temperature for 3 hours. The dichloromethane was removed by rotary evaporation under reduced pressure, and then a reaction solution was poured into 200 mL of a saturated saline solution, and extracted with 2×200 mL of ethyl acetate. An organic phase was concentrated to be semi-dry after rotary evaporation, purification for separation was continued using a silica gel chromatographic column, gradient elution was carried out once, an n-hexane solvent was used for washing first, then (n-hexane/ethyl acetate, 1:1, v/v, 1% triethylamine) were used for elution, (ethyl acetate/methanol 10:1, v/v, 1% triethylamine) continued to be used for elution, finally (ethyl acetate, 1% triethylamine) were used for elution, product components were collected, and a solvent was removed under reduced pressure to obtain a yellow solid intermediate 8 (8 g, 96%).

### Preparation of Intermediate A7′

The intermediate 8 (4.15 g, 4.5 mmol) was dissolved in 50 mL of dry dichloromethane, and then 2-cyanoethyl N,N,N′,N′-tetraisopropyl-phosphordiamidite (1.6 g, 5.4 mmol) was quickly added into the solution above. A reaction solution was stirred for 10 minutes in an ice bath under the protection of nitrogen. 11 mL of a 0.45 M tetrazolyl dichloromethane solution (5.2 mmol) was added into the reaction solution above, and the reaction solution continued to be stirred at room temperature under the protection of nitrogen to react for 3 hours. A reaction solution was poured into 100 mL of a saturated sodium bicarbonate solution, and extracted with 2×150 mL of ethyl acetate, an organic phase was separated out, anhydrous sodium sulfate was used for drying, and reduced pressure distillation was conducted to achieve a semi-dry state. Purification for separation was continued using a silica gel chromatographic column, gradient elution was carried out once, an n-hexane solvent was used for washing first, then (n-hexane/ethyl acetate, 1:1, v/v, 1% triethylamine) were used for elution, (n-hexane/ethyl acetate 1:2, v/v, 1% triethylamine) continued to be used for elution, finally (ethyl acetate, 1% triethylamine) were used for elution, product components were collected, and a solvent was removed under reduced pressure to obtain a white solid intermediate A7′ (3.9 g, 82%). ¹H NMR (CDCl₃): d, 7.43-7.41 (m, 4H, trityl), 7.32-7.27 (m, 12H, trityl), 7.25-7.17 (m, 2H, trityl), 6.83-6.79 (m, 8H, trityl), 3.78-3.73 (m, 2H), 3.61-3.59 (s, 12H), 3.0.5-3.01 (m, 4H), 2.80-2.78 (m, 1H), 2.63-2.60 (m, 2H), 2.22-2.16) m, 4H), 1.63-1.59 (m 12H), 1.40-1.36 (m, 4H), 1.28-1.22 (m, 2H), 1.20-1.17 (m, 14H) ppm. ³¹P NMR (CDCl₃): d, 148.77, 148.17 ppm.

### Example 4 Preparation of Oligonucleotides

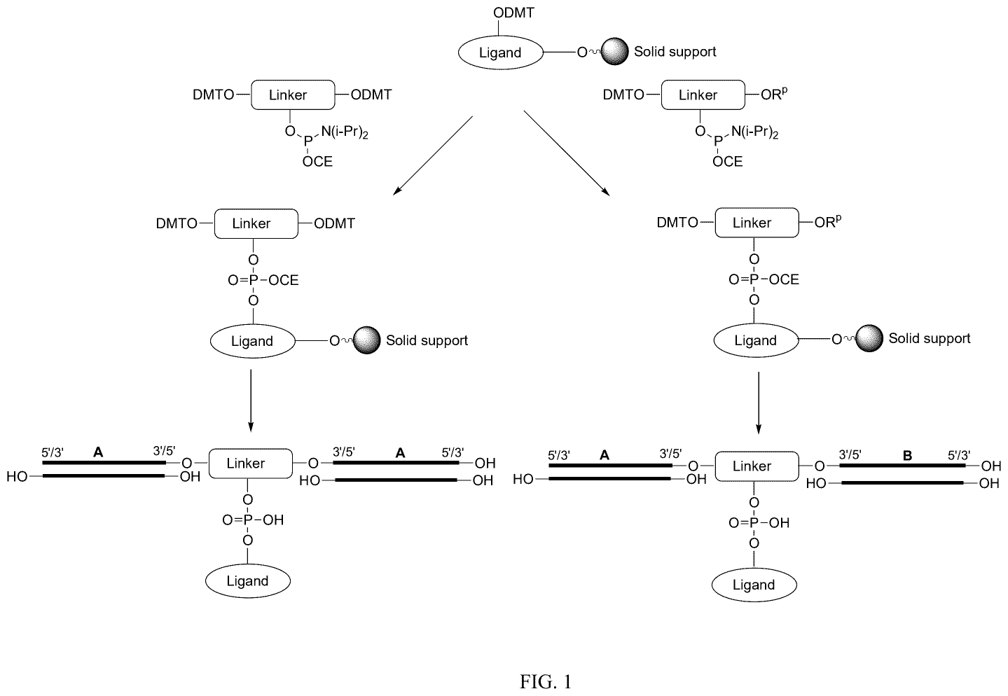

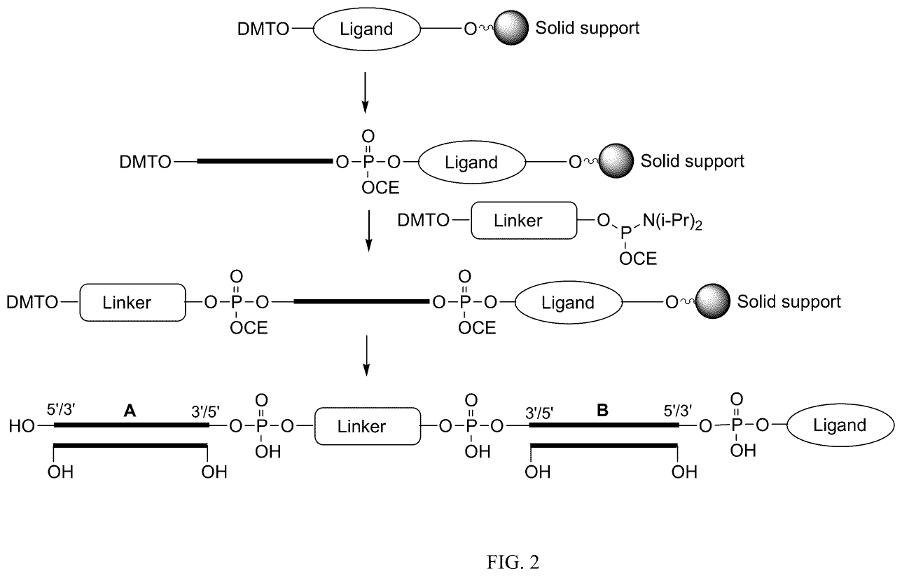

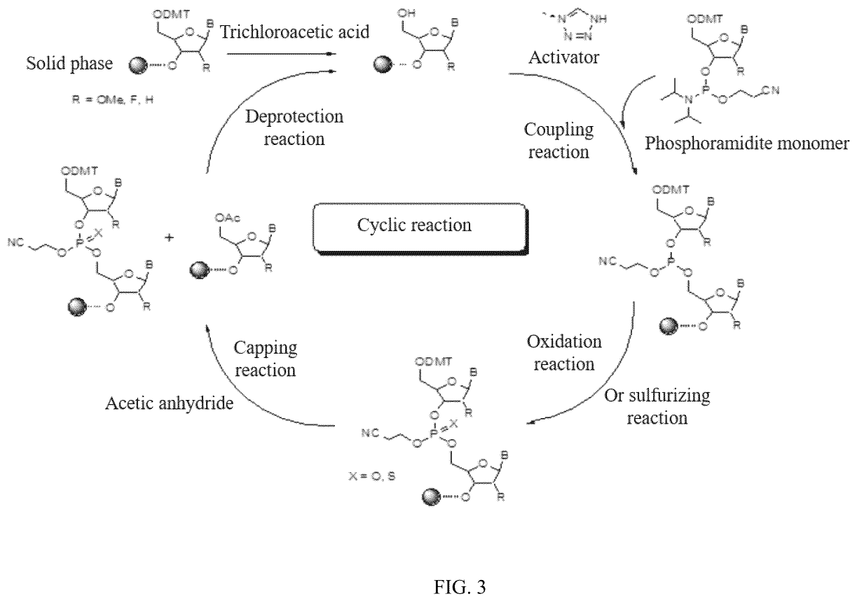

As shown in FIG. 1, according to sequences of oligonucleotides (such as an RNA sequence) in a compound to be prepared, a solid-phase phosphoramidite chemical synthesis method was adopted. Unless specially specified, unmodified or modified RNA phosphoramidite was commercially available. In specific embodiments, a solid support for solid-phase synthesis of a required nucleotide was selected from commercially-available general solid supports (NittoPhase® HL UnyLinker™ 300 Oligonucleotide Synthesis Support, Kinovate Life Sciences; or Long chain alkylamine controlled pore glass, CPG 500° A, 1000° A; Chemgenes, as shown below). A loading capacity of the solid support was typically a micro-mole number of a compound that could be linked and loaded per gram of the solid support (30-200 μmol/g).

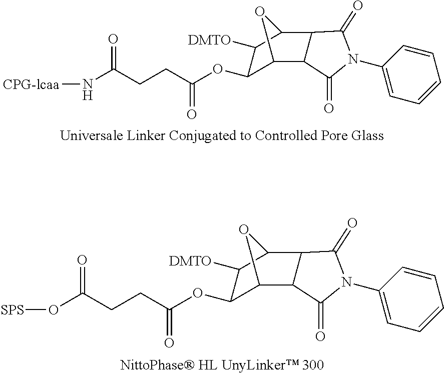

The oligonucleotides described in the present disclosure included active functional oligonucleotides, which could be selected from the following nucleic acid substances: small interfering RNA, microRNA, single-stranded RNA, antisense nucleic acids, inducing oligonucleotides, stem-loop RNA, etc. The functional oligonucleotides were composed of single-stranded oligonucleotides or double-stranded oligonucleotides. The small interfering RNA in the present disclosure was selected from the double-stranded oligonucleotides, including one sense strand and one antisense strand. The sense strand and the antisense strand were complementary, i.e., in a double-stranded nucleic acid molecule, bases of one strand formed hydrogen-bonded complementary pairs with those of the other strand. In double-stranded helical oligonucleotides, a purine base adenine (A) paired with a pyrimidine base thymine (T) or uracil (U); and a purine base guanine (G) always paired with a pyrimidine base cytosine (C). Sequences of two complementary strands were oriented from 5′- to 3′- for one strand and from 3′- to 5′- for the other strand. Each nucleotide in the small interfering RNA was independently either modified or unmodified. Modification referred to the substitution of 2′-hydroxy with other groups. Modification of a 2′-position of the nucleotide was possible selected from 2′-methoxy, 2′-fluoro, 2′-methoxyethyl, 2′-2,4-dinitrophenol, 2′-amino, 2′-4′-ethylene-bridged, and other groups. Nucleosides in the sequence were linked by phosphodiester linkages, where substitution of an oxygen atom in the phosphodiester linkage with a sulfur atom formed a phosphorothioate linkage. The sequence lengths of sense and antisense strands typically consisted of 19 (or 21) nucleotides and paired complementarily to form a duplex. A nucleoside sequence of the sense strand was a segment of nucleotide in a target mRNA. The antisense strand was usually linked with two consecutive deoxythymidine nucleotides or two consecutive uridine nucleotides. The target mRNA generally referred to an mRNA of a gene with abnormal protein expression in a cell.

### Example 5 Preparation of Compound Including Linker, Ligand and Oligonucleotides

In accordance with a phosphoramidite solid-phase synthesis method, following the order in the above sense strand(S) sequence, nucleoside monomers were linked one by one in a direction from 3′ to 5′. After linking to L1, the linking direction order of the nucleoside monomers was changed from “3′ to 5′” to “5′ to 3′”. The linking of each nucleoside monomer included a four-step reaction: deprotection, coupling, capping, and oxidation.

### Method for Preparing Solid-Phase Synthesis Reagent

A deprotection reagent was a dichloromethane solution (3%, v/v) of trichloroacetic acid or dichloroacetic acid. The nucleoside monomers were dissolved in anhydrous acetonitrile at a concentration of (0.05 M-0.1 M), with an appropriate amount of 3 Å molecular sieves added for dehydration treatment. A coupling activator was 5-ethylthio-1H-tetrazole in anhydrous acetonitrile at a concentration of (0.25 M or 0.45 M). 1H-tetrazole, 5-benzylthio-1H-tetrazole, or 4,5-dicyanoimidazole could also be selected as the activator. A capping reagent A was a tetrahydrofuran solution of acetic anhydride at a concentration of (10%, v/v). A capping reagent B was a mixed solvent with N-methylimidazole in pyridine and acetonitrile at a concentration of (15:10:75, v/v/v). An oxidation reagent was a water and pyridine solution of iodine (0.05 M, 95% pyridine aqueous solution). A sulfurizing reagent was (t-butylformyl)amino)-3H-1,2,4-dithiazoline-3-thione, at a concentration of (0.05M, pyridine/acetonitrile). A cleavage and deprotection reagent was 28% concentrated ammonia water.

### Reaction Condition Setup and Process for Solid-Phase Synthesis:

On a synthesizer, a molar ratio of a 4,4′-dimethoxytrityl protecting group on the solid support or a nucleoside monomer attached to the support to a dichloromethane solution of trichloroacetic acid (3%, v/v) was 1:30. A solid-phase reaction at room temperature lasted for 1.5 minutes and was repeated three times, and dropwise adding of the deprotection solution was stopped after the color of an eluent of the solid support was changed from red to be colorless. After repeated washing with anhydrous acetonitrile, the nucleoside monomer and the coupling activator (1:1) was added, where a molar ratio of the solid support to the nucleoside monomer was 1:5 to 1:6. The reaction between the reagents at room temperature and the solid phase proceeded for 3-4 minutes per cycle. After two cycles, the reaction was terminated. After washing with anhydrous acetonitrile, the oxidation reagent solution was added, where a molar ratio of the solid support to the oxidation reagent was 1:6. The oxidation reagent and the solid support reacted at room temperature for about 2 minutes, and the operation was repeated twice. After a coupling reaction, if a sulfurizing reaction step was required, the sulfurizing reagent solution was added, where a molar ratio of the solid support to the sulfurizing reagent was 1:6. The oxidation reagent and the solid support reacted at room temperature for about 4-5 minutes, and the operation was repeated twice. For a capping protection reaction, the capping reaction reagent was added, where a molar ratio of the solid support to the capping reagent was 1:80. The capping reagent and the solid support reacted at room temperature for about 1-2 minutes, and the operation was repeated twice. The above steps of deprotection, coupling, oxidation and capping were cycled until coupling of the last nucleotide was completed. The solid support carrying a sense strand or an antisense strand of a nucleic acid sequence was transferred into a vial, a 28% ammonia water was added, a glass cover was screwed for sealing, at a temperature of 55° C., base protecting groups in the sense strand or the antisense strand were hydrolyzed to be removed, and at the same time, the sense strand or the antisense strand was hydrolyzed and separated from the solid support. The reaction lasted for 16 hours. A resulting small nucleic acid sequence strand solution was filtered and separated from the solid support. A crude product of a small nucleic acid sequence strand was obtained after concentration.

### Purification and Separation by Preparative High Pressure Liquid Chromatography and Desalination

A preparative anion exchange chromatographic column (Source 15Q) was used, and small nucleic acids were purified through NaBr gradient elution. Mobile phase A: 20 mM sodium phosphate (pH 8.0), and mobile phase B: 20 mM sodium phosphate (pH 8.0), an aqueous solution of 1 M sodium bromide in 10% acetonitrile. The temperature of the column was 65° C. A flow rate was 10 mL/min. The elution gradient started from the mobile phase A, and then the mobile phase B was increased from 0% to 20% within 12 minutes. In the following 15 minutes, the mobile phase B was increased from 20% to 50%. A product eluent was collected, and component analysis and component integration were performed. Desalination was performed using a reversed-phase chromatographic purification column, or dialysis desalination was performed. Purified small nucleotides were obtained after concentration and freeze-drying. For the synthesized sense and antisense strands mentioned above, anion-exchange high-performance liquid chromatography (AEX-HPLC) was used to detect the purity, and reversed-phase liquid chromatography-mass spectrometry (LC-MS) was employed to determine and analyze a full-sequence molecular weight, confirming the synthesized nucleic acid sequence.

### Annealing Method

An implementation method was: the synthesized sense strand (S strand) and antisense strand (AS strand) were mixed at an equal molar ratio in normal saline for injection, heated at 90° C. for 5 minutes, then slowly cooled to a room temperature, and stored in a 4° C. refrigerator for 12 hours to allow the formation of a double-stranded structure via hydrogen bonding, yielding the siRNA-containing compound.

### Example 6 Preparation of SNK-2998

The structure of a compound SNK-2998 that could target FXI siRNA was as shown below:

Sense strand (S):
3′-RalsmUsmAsmUmUmUmAmCmAmGmAAfA
fCfmAAfmCmGmUmUsCfsmG-5′-A2-
5′ mGsCfsmUmUmGmCAfmACfAfAfmAmGm
AmCmAmUmUmUmAmU-3′-TriGalNac
Antisense strand (AS):
5′-mAsUfsmAmAmAUfmGUfmCmUmUmUmGU
fmUGfmCmAmAmGmCsmUsUf-3′

S: sense strand; AS: antisense strand
wherein the upper cases C, G, U and A represented base compositions of nucleotides; the lowercase m represented that an adjacent nucleotide on the right side of the letter m was a 2′-methoxy modified nucleotide; the lowercase f represented that an adjacent nucleotide on the left side of the letter f was a 2′-fluoro modified nucleotide; the lowercase s represented that two adjacent nucleotides on the left and right sides of the letter s were linked by a phosphorothioate linkage; and no other letter being present between two left and right adjacent nucleotides represented linking by a phosphodiester linkage. TriGalNac was a portion of Formula (603) remained after removal of Nu,

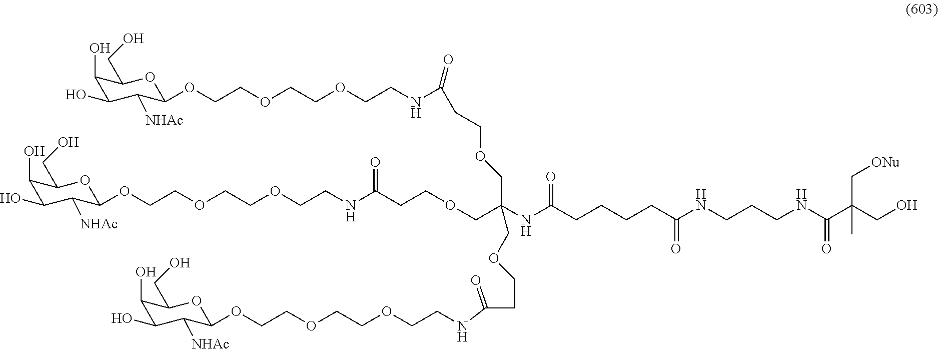

A2 was a linker with No. A2,
Ral was reverse abasic phosphoramidite shown below

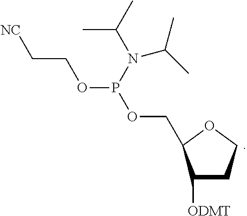

A phosphoramidite monomer required for synthesis from 3′ to 5′ was:
mC=5′-ODMT-2′-OMe-Cytidine-3′-Phosphoramidite (CAS: 199593-09-4)
mG=5′-ODMT-2′-OMe-Guanosine-3′-Phosphoramidite (CAS: 150780-67-9)
mA=5′-ODMT-2′-OMe-Adenosine-3′-Phosphoramidite (CAS: 11-782-31-5)
mU=5′-ODMT-2′-OMe-Uridine-3′-Phosphoramidite (CAS: 110764-79-9)
Cf=5′-ODMT-2′-Fluoro-Cytidine-3′-Phosphoramidite (CAS: 159414-99-0)
Gf=5′-ODMT-2′-Fluoro-Guanosine-3′-Phosphoramidite
Af=5′-ODMT-2′-Fluoro-Adenosine-3′-Phosphoramidite (CAS: 136834-22-5)
Uf=5′-ODMT-2′-Fluoro-Adenosine-3′-Phosphoramidite

A phosphoramidite monomer required for synthesis from 5′ to 3′ was:
mC=3′-ODMT-2′-OMe-Cytidine-5′-Phosphoramidite
mG=3′-ODMT-2′-OMe-Guanosine-5′-Phosphoramidite
mA=3′-ODMT-2′-OMe-Adenosine-5′-Phosphoramidite
mU=3′-ODMT-2′-OMe-Uridine-5′-Phosphoramidite
Cf=3′-ODMT-2′-Fluoro-Cytidine-5′-Phosphoramidite
Gf=3′-ODMT-2′-Fluoro-Guanosine-5′-Phosphoramidite
Af=3′-ODMT-2′-Fluoro-Adenosine-5′-Phosphoramidite
Uf=3′-ODMT-2′-Fluoro-Adenosine-5′-Phosphoramidite

All the monomers above were purchased from Shanghai Hongene Biotech Co., Ltd. The above monomers were placed in an RNA/DNA automated synthesizer (MerMode-12) for synthesis.

### Experimental Example 1

### Evaluation on Effect of Compound SNK-3464 Targeting AGT (Angiotensinogen)

Human AGT transgenic mice (n=3) were subcutaneously injected with 3 mg/kg of SNK-3464 on day 0. Serum samples were collected and analyzed for hAGT protein levels by ELISA.

This experimental example corresponded to a situation shown in Scheme I.

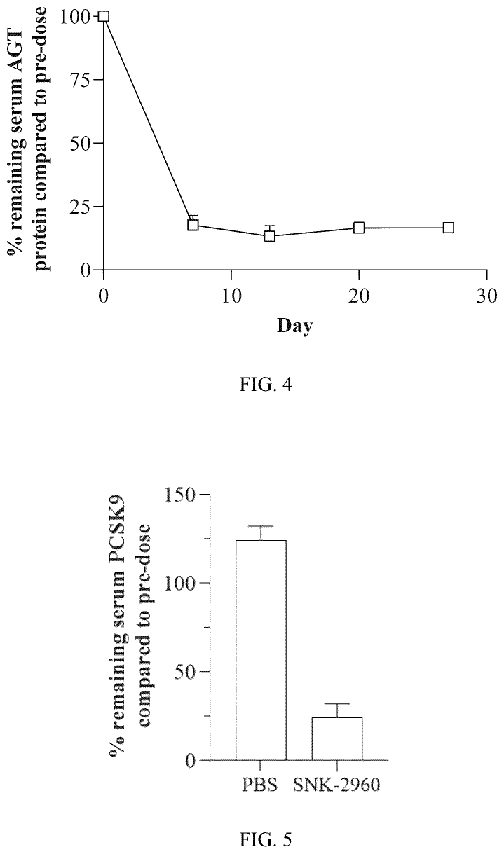

The results were as shown in FIG. 4. It could be clarified according to FIG. 4 that, binding of the compound of the present disclosure to AGT significantly inhibited AGT.

### Experimental Example 2

### Evaluation on Effect of Compound SNK-2960 Targeting PCSK9

SNK-2960 was prepared at 6 mg/kg dose and subcutaneously injected into human PCSK9 transgenic mice on day 0. Serum PCSK9 protein levels on day 7 were monitored by ELISA.

This experimental example corresponded to a situation shown in Scheme III.

The results were as shown in FIG. 5. It could be clarified according to FIG. 5 that, binding of the compound of the present disclosure to PCSK9 significantly inhibited PCSK9.

### Experimental Example 3

### Evaluation on Effect of Compound SNK-2998 Targeting FXI

Cynomolgus monkeys (n=3) were subcutaneously administered with SNK-2998 on Day 0 and plasma samples were collected. FXI protein levels were measured by ELISA (Abcam, ab137973).

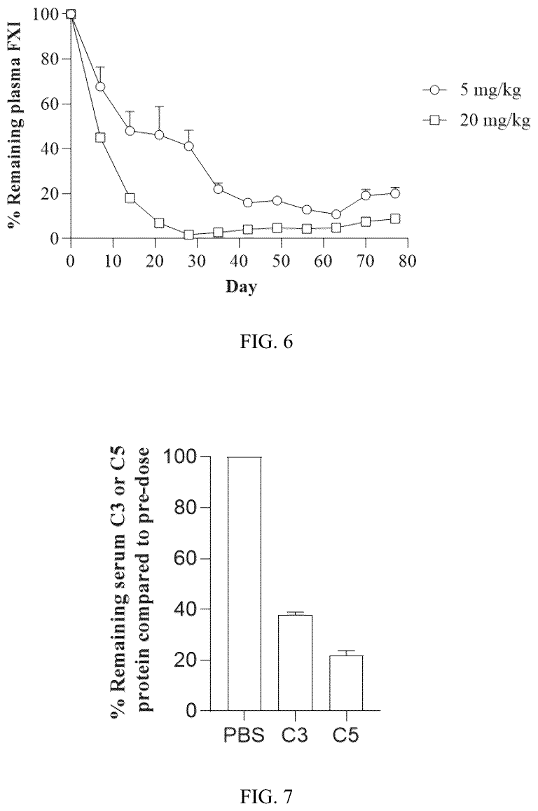

FIG. 6 showed dose-dependent inhibition of FXI protein levels. Monkeys receiving 20 mg/kg dose showed a maximum inhibition of about 99% and inhibition lasted for more than two months.

This experimental example corresponded to a situation shown in Scheme III.

### Experimental Example 4

### Evaluation on Effect of Compound SNK-3467 Targeting C3 or C5

In some disease areas where multiple factors contribute to the disease progression such as PCSK9 regulates LDL-c level and ANGPTL3 regulates TG level, targeting both PCSK9 and ANGPTL3 at the same time may provide beneficial treatment effects. Same is true for LPA and PCSK9 where both may contribute to cardiovascular diseases. Therefore, targeting both LPA and PCSK9 can provide a better therapeutic option than targeting either one alone. In some cases, individuals can have hypertension and high cholesterol at the same time. By targeting AGT and PCSK9 simultaneously can also be beneficial. Another potential therapeutic area is complement related diseases where targeting one complement component may not provide adequate protection or cannot completely block the activation of complement cascade (Harder M J et al., 2016, Blood 129:970-980). Targeting multiple complement components such as C3, CFB and C5 can provide better complement inhibition than either one alone. In such design, one could have one siRNA targeting C5, a terminal, lytic pathway complement component and another siRNA targeting C3, a central complement that is involved in classical pathway, lectin pathway and alternative pathway. By targeting both C3 and C5, one could potentially block the residual complement activity while targeting either one alone.

SNK-3467 was subcutaneously injected into either human C3 or human C5 transgenic mice at 6 mg/kg on day 0 and serum C3 or C5 were evaluated by ELISA on day 7. As shown in FIG. 7, SNK-3467 howed gene silencing effect on both C3 and C5.

This experimental example corresponded to a situation shown in Scheme V.

### Experimental Example 5

Evaluation on Effect of Compound SNK-3010 Targeting AGT and hPCSK9

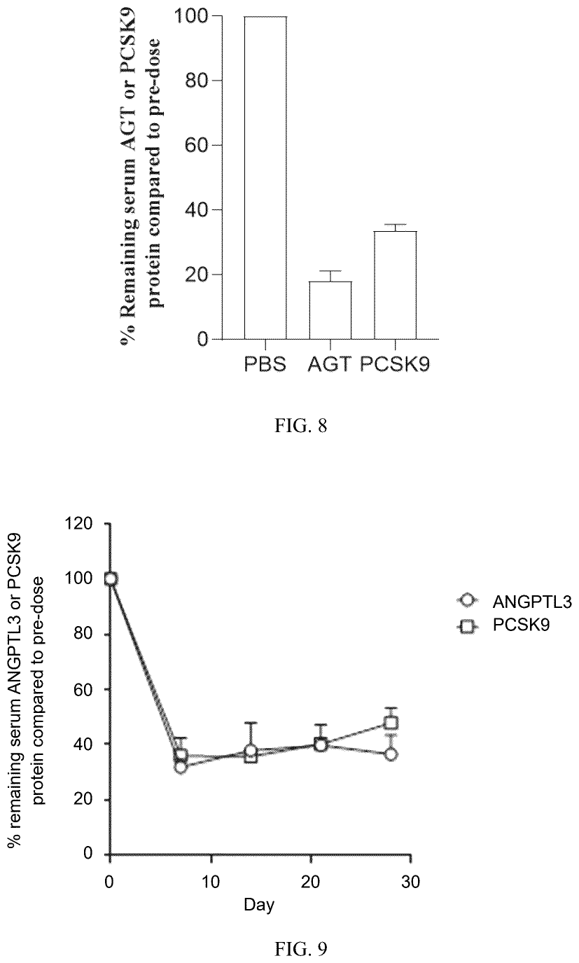

SNK-3010 was subcutaneously injected at a dose of 6 mg/kg to either hAGT or hPCSK9 transgenic mice on day 0. Serum hAGT and hPCSK9 levels were evaluated by ELISA on day 7. As shown in FIG. 8, SNK-3010 can also simultaneously silence the expression of hAGT and hPCSK9.

This experimental example corresponded to a situation shown in Scheme V.

### Experimental Example 6

Evaluation on Effect of Compound SNK-3005 Targeting hANGPTL3 and hPCSK9

Human ANGPTL3 or PCSK9 transgenic mice (n=3) were subcutaneously injected with 7.7 mg/kg of SNK-3005 on day 0. Serum samples were collected and analyzed for hANGPTL3 or hPCSK9 protein levels by ELISA.

The results were as shown in FIG. 9. It could be clarified according to FIG. 9 that, binding of the compound of the present disclosure to hANGPTL3 and hPCSK9 significantly inhibited hANGPTL3 and hPCSK9.

### Experimental Example 7

Evaluation on Effect of Compound SNK-3461 Targeting hANGPTL3 and hPCSK9

Human ANGPTL3 or PCSK9 transgenic mice (n=3) were subcutaneously injected with 7.7 mg/kg of SNK-3461 on day 0. Serum samples were collected and analyzed for hANGPTL3 or hPCSK9 protein levels by ELISA.

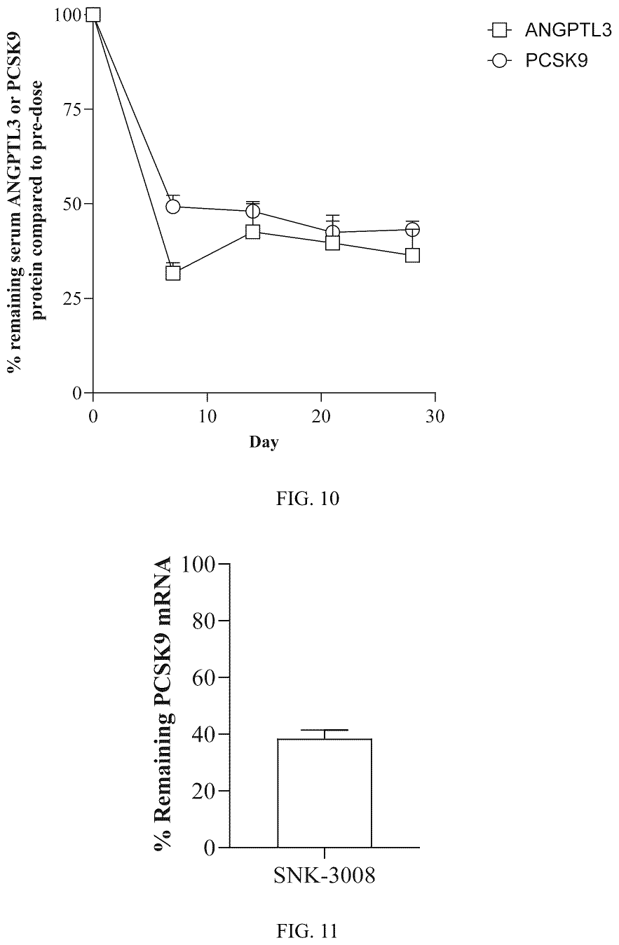

The results were as shown in FIG. 10. It could be clarified according to FIG. 10 that, binding of the compound of the present disclosure to hANGPTL3 and hPCSK9 significantly inhibited hANGPTL3 and hPCSK9.

### Experimental Example 8

### Evaluation on Effect of Compound SNK-3008 Targeting PCSK9

Hep3B cells were transfected with 100 pM SNK-3008 plus Lipofectamine® RNAiMAX (ThermoFisher) in duplicate and incubated for 48 hours. Total RNA was extracted using RNAeasy Mini kit (QIAGEN). PCSK9 mRNA levels were determined by RT-qPCR and compared to Lipofectamine® RNAiMAX alone samples.

The results were as shown in FIG. 11. It could be clarified according to FIG. 11 that, binding of the compound of the present disclosure to PCSK9 significantly inhibited PCSK9.

### Experimental Example 9

### Evaluation on Effect of Compound SNK-3468 Targeting CFB and PCSK9

SNK-3468 was prepared at 12 mg/kg dose and subcutaneously injected into human CFB or PCSK9 transgenic mice (n=3) on day 0. Serum CFB or PCSK9 protein levels on day 7 were monitored by ELISA.

This experimental example corresponded to a situation shown in Scheme XV.

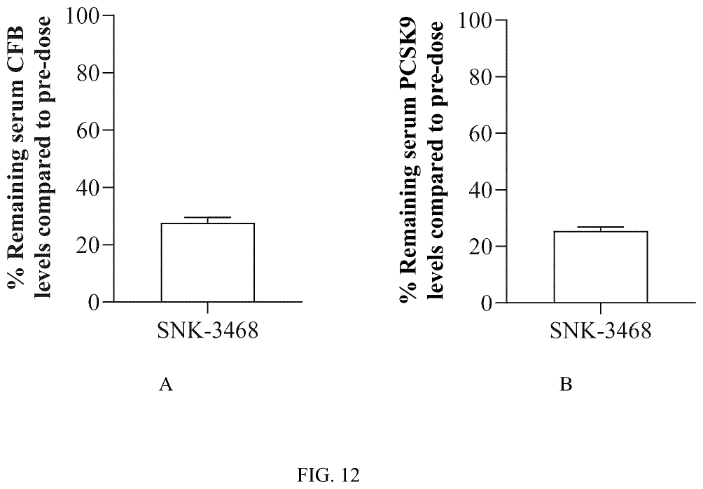

The results were as shown in FIG. 12. It could be clarified according to FIG. 12 that, binding of the compound of the present disclosure to CFB and PCSK9 significantly inhibited CFB and PCSK9.

### Experimental Example 10

### Comparison of Linker UsUUsU in the Prior Art and Linker A2 of the Present Disclosure

Hep3B cells were transfected with 100 pM siRNAs (SNK-3466 or SNK-2192) plus Lipofectamine® RNAiMAX (ThermoFisher) in duplicate and incubated for 48 hours. Total RNA was extracted using RNAeasy Mini kit (QIAGEN). PCSK9 and AGT mRNA levels were determined by RT-qPCR and compared to Lipofectamine® RNAiMAX alone samples.

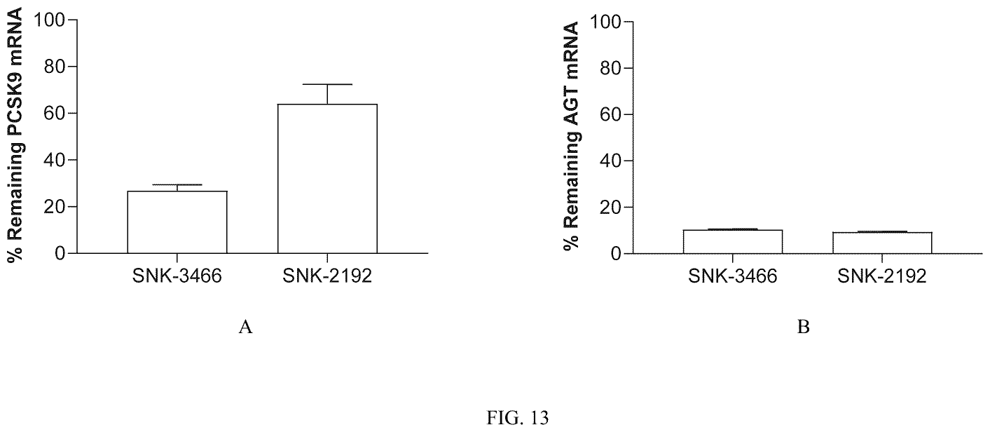

The results were as shown in FIG. 13. It could be clarified according to FIG. 13 that, binding of the compound of the present disclosure to PCSK9 and AGT significantly inhibited PCSK9 and AGT. Compared with the known linker, the linker of the present disclosure had the significantly better effect for PCSK9.

### Experimental Example 11

### Comparison of Compound SNK-2959 Including Other Linkers and Compound SNK-2960 Including Linker A2 of the Present Disclosure

The structure of Linker in SNK-2959 is

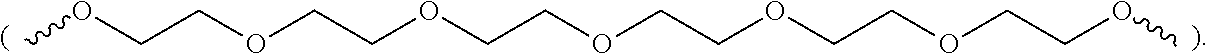

It was prepared using a compound purchased from Hongene Biotech (CAS No.: 125607-09-2).

SNK-2959 and SNK-2960 were prepared at 6 mg/kg dose and subcutaneously injected into human PCSK9 transgenic mice on day 0. Serum PCSK9 protein levels on day 7 were monitored by ELISA.

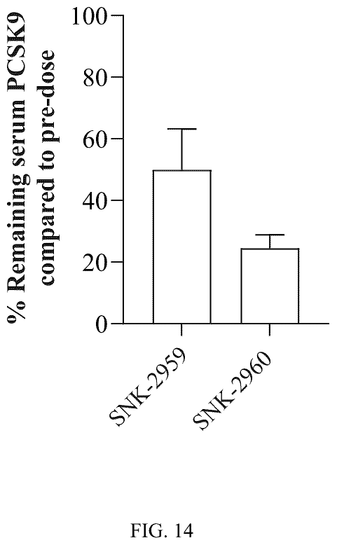

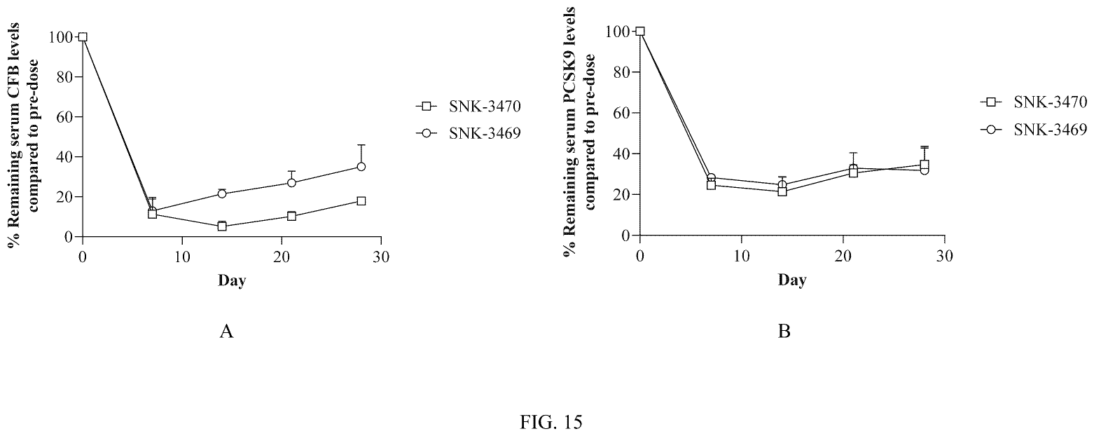

The results were as shown in FIG. 14. It could be clarified according to FIG. 14 that, binding of the compound of the present disclosure to PCSK9 significantly inhibited PCSK9. Compared with other linkers, the linker of the present disclosure had the significantly better effect for PCSK9.

### Experimental Example 12

### Evaluation on Influence of Oligonucleotide Structure Modification on Activity

Human CFB or PCSK9 transgenic mice (n=3) were subcutaneously injected with 6 mg/kg SNK-3469 or SNK-3470 on day 0. Serum samples were collected and analyzed for hCFB or hPCSK9 protein levels by ELISA.

The results were as shown in FIG. 14. It could be clarified according to FIG. 14 that, binding of the compounds of the present disclosure to both CFB and PCSK9 significantly inhibited CFB and PCSK9. When the oligonucleotides in the compounds of the present disclosure were modified with Ral, the compounds had the significantly better effect for CFB.

Obviously, the above examples are only instances for clear explanation, rather than limiting the embodiments. For those of ordinary skill in the art, other different forms of changes or variations can further be made based on the above explanation. It is not necessary and impossible to exhaustively list all embodiments here. The obvious changes or variations arising from this are still within the scope of protection of the present disclosure.

While this invention has been particularly shown and described with references to preferred embodiments thereof, it will be understood by those skilled in the art that various changes in form and details may be made therein without departing from the scope of the invention encompassed by the appended claims.

1. A linker, comprising:
a structure selected from the group consisting of: 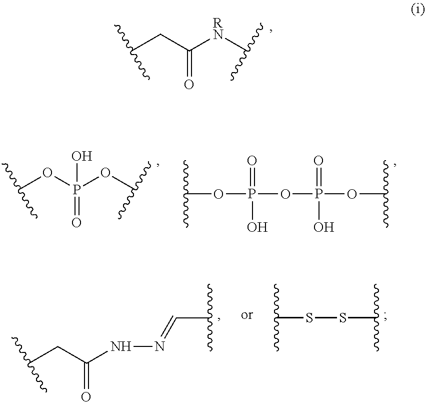
(ii) a combination of 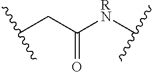
and one or more of 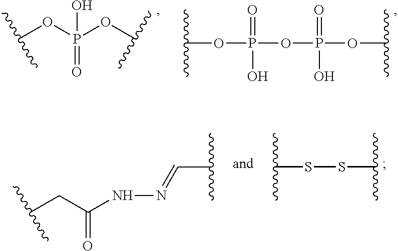
or
(iii) a combination of 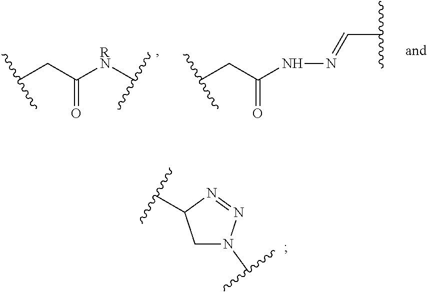
wherein R is selected from —H, —CO(CH₂)_{d}CH₃, —(CH₂)_{d}CH₃, —CH(CH₃)₂, —(CH₂)_{d}OH, —(CH₂)_{d}COOH, —CO(CH₂)_{d}CH₃, —(CH₂)_{d}CONH₂, —(CH₂)_{d}NHCOH, —(CH₂)_{d}NH₂, —(CH₂)_{d}NHCO(CH₂)ₑCH₃, or —(CH₂)_{d}CONH(CH₂)ₑCH₃, wherein d and e are independently selected from integers ranging from 0 to 20; the linker is hydrolysable.

2. A linker comprising a structure selected from the group consisting of formulas A, B, C, E, and F, as shown below: 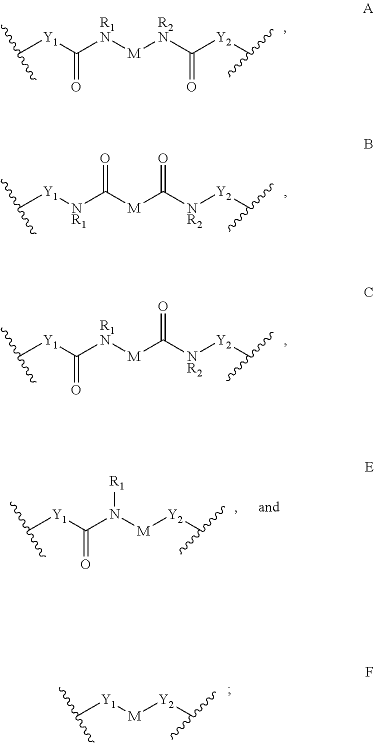
wherein M is selected from the group consisting of: a C1-C30 alkylidene group, a substituted C1-C30 alkylidene group, a C2-C30 alkenylidene group, a substituted C2-C30 alkenylidene group, a C2-C30 alkynylene group, a substituted C2-C30 alkynylene group, a nitrogen-containing functional group, a phosphorus-containing functional group, an oxygen-containing functional group, a sulfur-containing functional group, a silicon-containing functional group, and a gold-containing functional group; wherein M is optionally attached to an oligonucleotide and/or a ligand;
Y₁ and Y₂ are independently selected from —[CH₂]_{m′}(CHQ′)[CH₂]_{n′}—, —(CH₂)_{m′}NQ′(CH₂)_{n′}—, —(CH₂)_{m′}OP(═O)(X′)O(CH₂)_{n′}—, —(CH₂)ₘ(OCH₂CH₂)_{t′}O(CH₂)_{n′}—, —(CH₂)_{m′}(CH═CH)_{t′}(CH₂)_{n′}—, —(CH₂)_{m′}(C₆H₄)(CH₂)_{n′}—, —(CH₂)_{m′}CO(CH₂)_{n′}—, —(CH₂)_{m′}(C₆H₁₀)(CH₂)_{n′}—, —(CH₂)_{m′}(C₆H₁₀O₆)(CH₂)_{n′}—, —(CH₂)_{m′}—O—SiO—O—(CH₂)_{n′}—, —[CH₂]_{m′}(CHQ′)[CH₂]_{n′}O—, —(CH₂)_{m′}NQ′ (CH₂)_{n′}O—, —(CH₂)_{m′}OP(═O)(X′)O(CH₂)_{n′}O—, —(CH₂)_{n′}OP(═O)OHO—, —(CH₂)_{n′}OP(═S)OHO—, —(CH₂)_{m′}S—S(CH₂)_{n′}O—, —(CH₂)_{m′}(CH═CH)_{t″}(CH₂)_{n′}O—, —(CH₂)_{m′}(C₆H₄)(CH₂)_{n′}O—, —(CH₂)_{m′}CO(CH₂)_{n′}O—, —(CH₂)_{m′}(C₆H₁₀)(CH₂)_{n′}O—, —(CH₂)_{m′}(C₆H₁₀O₆)(CH₂)_{n′}O—, —(CH₂)_{m′}—O—SiO—O—(CH₂)_{n′}O—, —[CH₂]_{m′}(CHQ′)[CH₂]_{n′}S—, —(CH₂)_{m′}NQ′(CH₂)_{n′}S—, —(CH₂)_{m′}OP(═O)(X′)O(CH₂)_{n′}S—, —(CH₂)_{m′}S—S(CH₂)_{n′}S—, —(CH₂)_{m′}(CH═CH)_{t′}(CH₂)_{n′}S—, —(CH₂)_{m′}(C₆H₄)(CH₂)_{n′}S—, —(CH₂)_{m′}CO(CH₂)_{n′}S—, —(CH₂)_{m′}(C₆H₁₀)(CH₂)_{n′}S—, —(CH₂)_{m′}(C₆H₁₀O₆)(CH₂)_{n′}S—, —(CH₂)_{m′}—O—SiO—O—(CH₂)_{n′}S—, 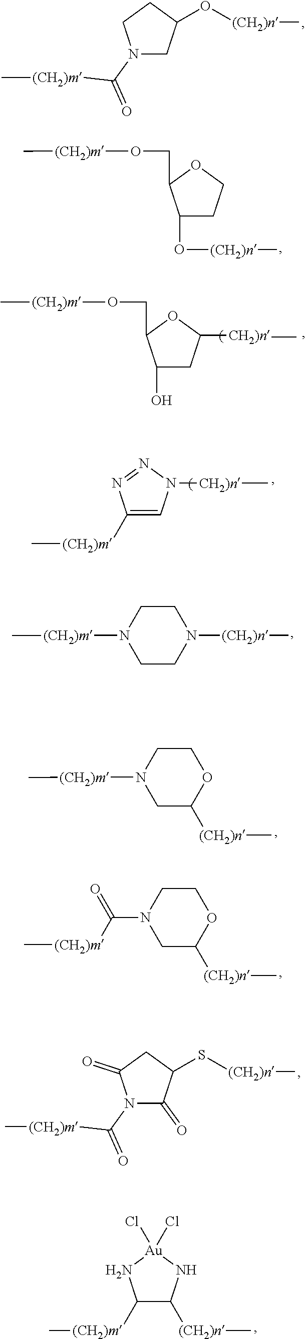 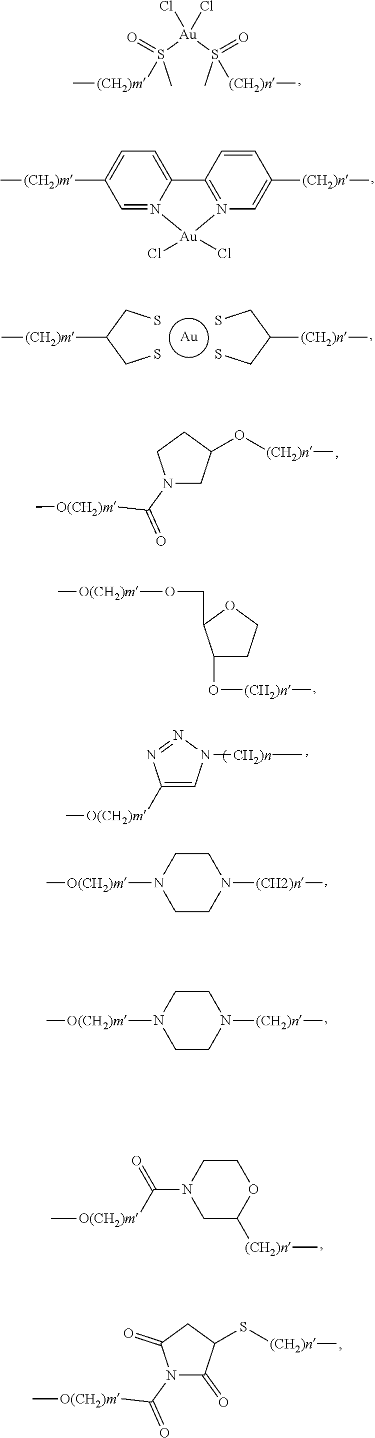 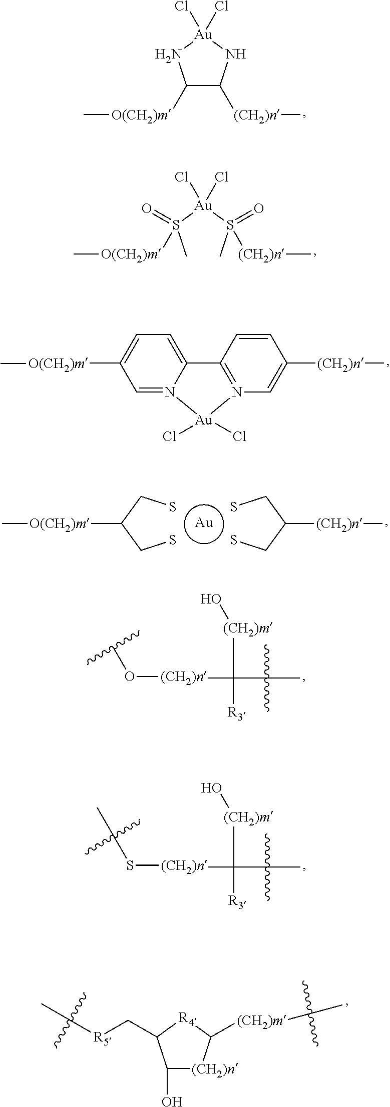
—(CH₂)_{m′}O(CH₂)_{n′}O—, —(CH₂)_{m′}OP(═S)(Y′)O(CH₂)_{n′}—, —(CH₂)_{m′}NHCO(CH₂)_{n′}—, —(CH₂)_{m′}O—(CH₂)_{n′}—, —(CH₂)(OCH₂CH₂)_{n′}O(CH₂)_{m′}CONH—, 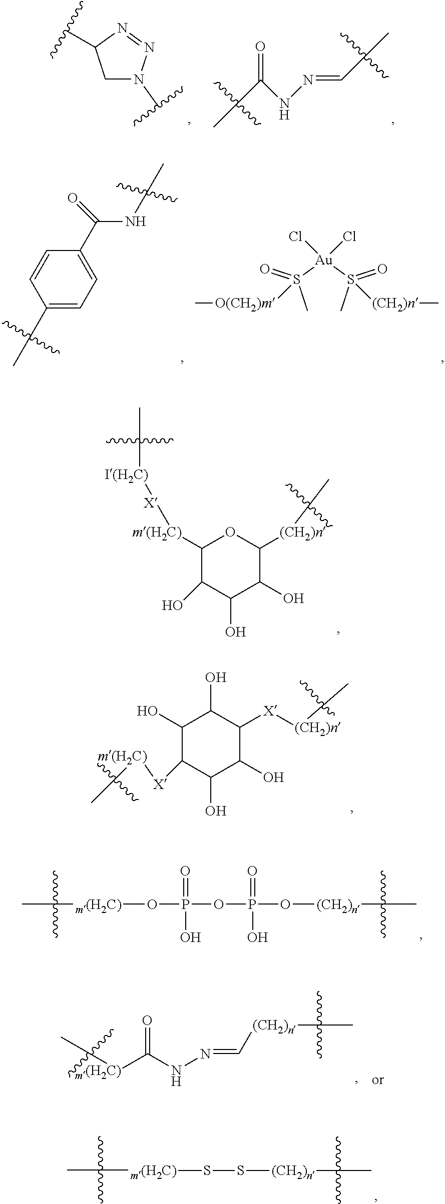
wherein l′, n′, m′, and t′ are independently selected from the integers ranging from 0 through 20; wherein Y₁ and Y₂ are the same or different;
R_{3′} is selected from H, —Cl, —Br, —F, —OH, —(CH₂)_{x′}CH₃, or —O(CH₂)_{x′}CH₃; wherein x′ is an integer ranging from 0 to 20;
R_{4′} and R_{5′} are independently selected from —O—, —S—, —CH₂—, —NH—, —HCH₃—, or —NCH₃—;
X′ and Y′ are each independently selected from OH, SH, O, O⁻, S, S⁻, —NH₂, —CH₃, or —OCH₃;
Q′ is selected from —H, —F, —Cl, —Br, —I, —(CH₂)_{a′}CH₃, —CH(CH₃)₂, —(CH₂)_{a′}OH, —(CH₂)_{a′}COOH, —CO(CH₂)_{a′}CH₃, —(CH₂)_{a′}NHCO(CH₂)_{b′}O—, —(CH₂)_{a′}CONH(CH₂)_{b′}O—, —(CH₂)_{a′}CONH—NH═CH(CH₂)_{b′}—, —(CH₂)_{a′}CONH—NH═CH(CH₂)_{b′}O—, —(CH₂)_{a′}CO(CH₂)_{b′}O—, —(CH₂)_{a′}CONH₂, —(CH₂)_{a′}NHCOH, —(CH₂)_{a′}NH₂, —(CH₂)_{a′}NHCO(CH₂)_{b′}CH₃, —(CH₂)_{a′}CONH(CH₂)_{b′}CH₃, —O(CH₂)_{a′}O—, —(CH₂)_{a′}(OCH₂CH₂)_{b′}O(CH₂)_{c′}—, —OPO₃H—, —O—PSO₂H—, —OP(═O)(OH)₂, —OP(═S)(OH)₂, —OP(═O)(CH₂)_{a′}CH₃OH, —OP(═O)(NH₂)OH, —OP(═O)[O(CH₂)_{a′}CH₃]OH, —OP(═O)[NH(CH₂)_{a′}CH₃]OH, —OP(═S)(CH₂)_{n′}CH₃OH, —OP(═S)[O(CH₂)_{n′}CH₃]OH, —OP(═S)(NH₂)OH, —OP(═S)ₙH(CH₂)_{a′}CH₃OH, —OP(═S)SHOH, —OS(═O)₂OH, —OS(═O)OH, —(CH₂)_{a′}OP(═O)OHO—, —(CH₂)_{a′}OP(═S)OHO—, —OP(═O)(CH₂)_{a′}CH₃O—, —OP(═O)(OCH₃)O—, —OP(═O)(NH₂)O—, —(CH₂)_{a′}O—, —(OCH₂CH₂)_{a′}—O—, —(CH═CH—CH₂)_{a′}O—, —(C₆H₄—CH₂—)_{a′}O—, —(C₆H₁₀)_{a′}O—, —(C₆H₁₀—CH₂—)_{a′}O—, wherein a′, b′, and c′ are independently selected from integers ranging from 0 through 20;
R₁ and R₂ are independently selected from —H, —CO(CH₂)_{d}CH₃, —(CH₂)_{d}CH₃, —CH(CH₃)₂, —(CH₂)_{d}OH, —(CH₂)_{d}COOH, —CO(CH₂)_{d}CH₃, —(CH₂)_{d}CONH₂, —(CH₂)_{d}NHCOH, —(CH₂)_{d}NH₂, —(CH₂)_{d}NHCO(CH₂)ₑCH₃, or —(CH₂)_{d}CONH(CH₂)·CH₃, wherein d and e are independently selected from integers ranging from 0 to 20.

3. The linker according to claim 2, wherein M has a symmetrical structure.

4. The linker according to claim 2, wherein R₁ and R₂ are independently selected from —H or —(CH₂)_{d}CH₃.

5. The linker according to claim 2, wherein M is independently selected from —[CH₂]ₘ(CHQ)[CH₂]ₙ—, —(CH₂)ₘNQ(CH₂)ₙ, —(CH₂)ₘOP(═O)(X)O(CH₂)ₙ, —(CH₂)ₘ(OCH₂CH₂)ₜO(CH₂)ₙ, —(CH₂)ₘ(CH═CH)ₜ(CH₂)ₙ, —(CH₂)ₘ(C₆H₄)(CH₂)ₙ, —(CH₂)ₘCO(CH₂)ₙ, —(CH₂)ₘ(C₆H₁₀)(CH₂)ₙ, —(CH₂)ₘ(C₆H₁₀O₆)(CH₂)ₙ, —(CH₂)ₘ—O—SiO—O—(CH₂)ₙ, —[CH₂]ₘ(CHQ)[CH₂]ₙO, —(CH₂)ₘNQ(CH₂)ₙO, —(CH₂)ₘOP(═O)(X)O(CH₂)ₙO, —(CH₂)ₙOP(═O)OHO, —(CH₂)ₙOP(═S)OHO, —(CH₂)ₘS—S(CH₂)ₙO, —(CH₂)ₘ(CH═CH)ₜ(CH₂)ₙO, —(CH₂)ₘ(C₆H₄)(CH₂)ₙO, —(CH₂)ₘCO(CH₂)ₙO, —(CH₂)ₘ(C₆H₁₀)(CH₂)ₙO, —(CH₂)ₘ(C₆H₁₀O₆)(CH₂)ₙO, —(CH₂)ₘ—O—SiO—O—(CH₂)ₙO, —[CH₂]ₘ(CHQ)[CH₂]ₙS, —(CH₂)ₘNQ (CH₂)ₙS, —(CH₂)ₘOP(═O)(X)O(CH₂)ₙS, —(CH₂)ₘS—S(CH₂)ₙS, —(CH₂)ₘ(CH═CH)ₜ(CH₂)ₙS, —(CH₂)ₘ(C₆H₄)(CH₂)ₙS, —(CH₂)ₘCO(CH₂)ₙS, —(CH₂)ₘ(C₆H₁₀)(CH₂)ₙS, —(CH₂)ₘ(C₆H₁₀O₆)(CH₂)ₙS, —(CH₂)ₘ—O—SiO—O—(CH₂)ₙS, 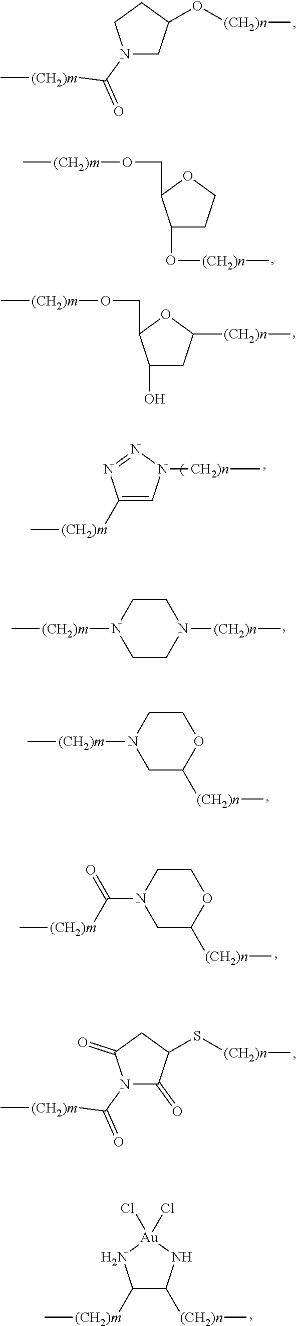 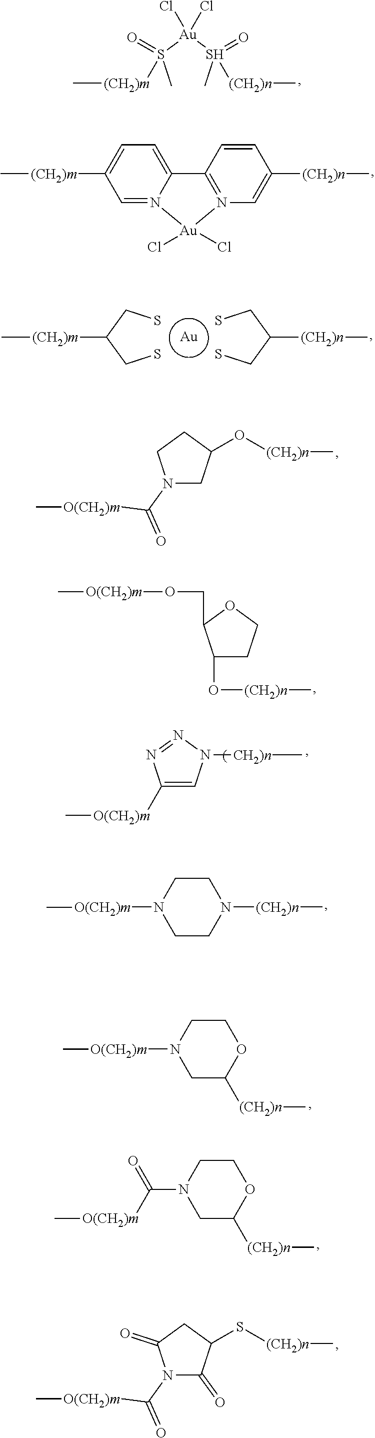 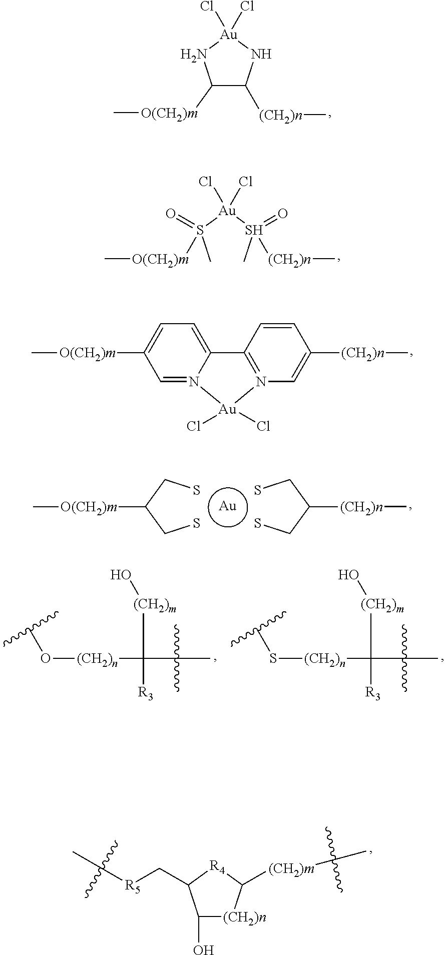
—(CH₂)ₘO(CH₂)ₙO, —(CH₂)ₘOP(═S)(Y)O(CH₂)ₙ, —(CH₂)ₘNHCO(CH₂)ₙ, —(CH₂)ₘO—(CH₂)ₙ, —(CH₂)ₗ(OCH₂CH₂)ₙO(CH₂)ₘCONH, 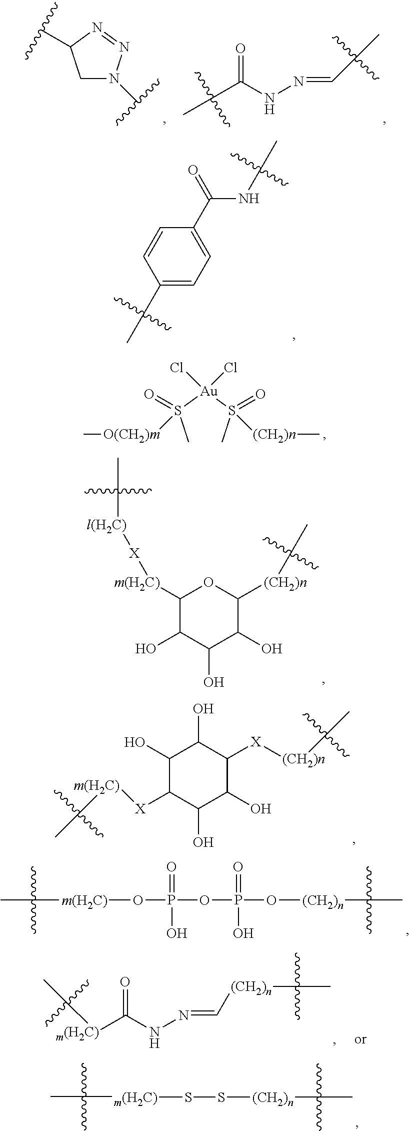
wherein l, n, m, and t are independently selected from integers ranging from 0 through 20.

6. The linker according to claim 5, wherein M is selected from —(CH₂)ₘNQ(CH₂)ₙ—, —[CH₂]ₘ(CHQ)[CH₂]ₙ—, —(CH₂)ₘ(CH═CH)ₜ(CH₂)ₙ—, —(CH₂)ₘOP(═O)(X)O(CH₂)ₙ—, —(CH₂)ₘOP(═S)(Y)O(CH₂)ₙ—, —(CH₂)ₘ(OCH₂CH₂)ₜO(CH₂)ₙ—, —(CH₂)ₘ—O—SiO—O—(CH₂)ₙ—, 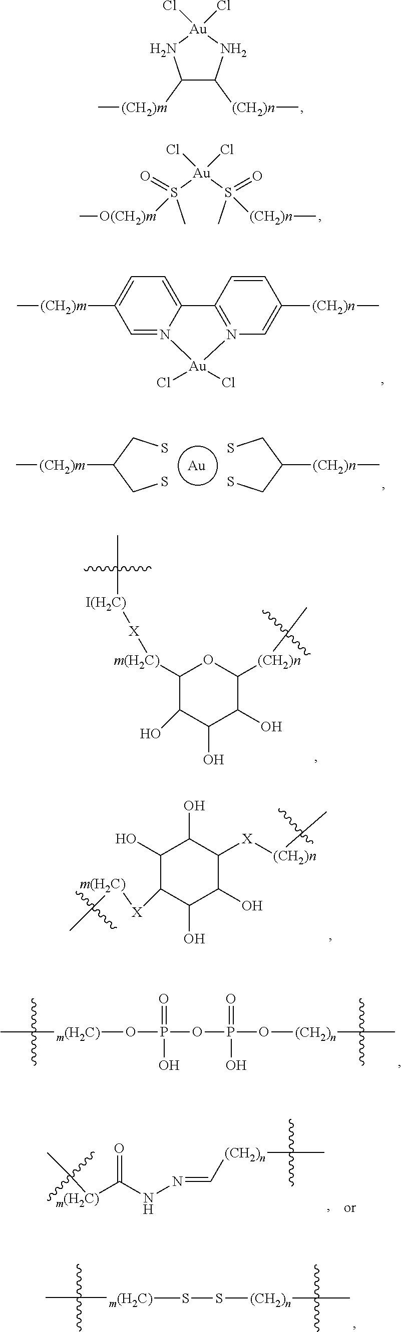
wherein Q is —(CH₂)ₐ(OCH₂CH₂)_{b}O(CH₂)_{c}—, —(CH₂)ₐCH₃, —(CH₂)ₐCO(CH₂)_{b}O—, or —(CH₂)ₐCONH(CH₂)_{b}O—, wherein l, n, m, t, a, b, and c independently are selected from integers ranging from 0 to 20, and X and Y are each independently selected from OH, SH, O, O⁻, S, S⁻, —NH₂, —CH₃, or —OCH₃.

7. The linker according to claim 5, wherein M is selected from:
(a) M is a C1-C30 alkylidene group,
(b) when M is —[CH₂]ₘ(CHQ)[CH₂]ₙ—, Q is —(CH₂)_{a′}(OCH₂CH₂)_{b}O(CH₂)_{c}—, a, b, and c are each 0, and neither m nor n is 0;
(c) when M is —(CH₂)ₘ(CH═CH)ₜ(CH₂)ₙ—, neither m, n nor t is 0;
(d) when M is —(CH₂)ₘNQ(CH₂)ₙ—, neither m nor n is 0, Q is —(CH₂)_{d}CH₃, —(CH₂)ₐCO(CH₂)_{b}O—, or —(CH₂)ₐCONH(CH₂)_{b}O—;
(e) when M is —(CH₂)ₘOP(═O)(X)O(CH₂)ₙ—, neither m nor n is 0, X is OH, SH, O⁻ or S⁻;
(f) when M is —(CH₂)ₘOP(═S)(Y)O(CH₂)ₙ—, neither m nor n is 0, and Y is selected from OH, SH, O⁻ or S⁻;
(g) when M is —(CH₂)ₘ(OCH₂CH₂)ₜO(CH₂)ₙ—, neither m nor n nor t is 0;
(h) when M is 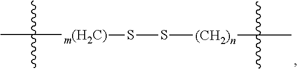
neither m nor n is 0;
(i) when M is —(CH₂)ₘ—O—SiO—O—(CH₂)ₙ—, neither m nor n is 0;
(j) when M is 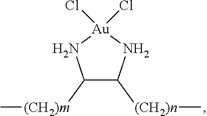
neither m nor n is 0;
(k) when M is 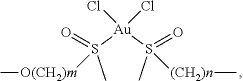
neither m nor n is 0;
(l) when M is 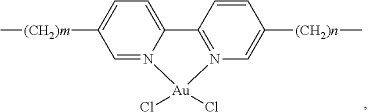
neither m nor n is 0;
(m) when M is 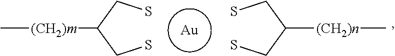
neither m nor n is 0;
(n) when M is 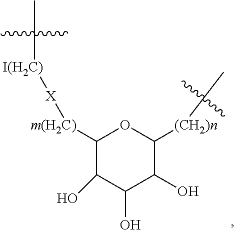
X is O or S, neither l, m, nor n is 0;
(o) when M is 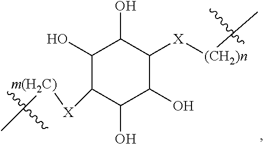
X is O or S, neither m nor n is 0;
(p) when M is 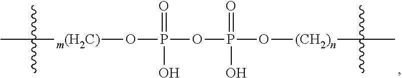
neither m nor n is 0; or
(q) when M is 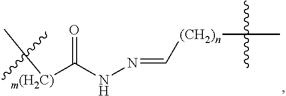
neither m nor n is 0.

8. The linker according to claim 7, wherein:
(a) when M is —(CH₂)ₘNQ (CH₂)ₙ—,
(i) if Q is —(CH₂)ₐCO(CH₂)_{b}O—, then a is 0 or a non-zero integer, and b is a non-zero integer; and
(ii) if Q is —(CH₂)ₐCONH(CH₂)_{b}O—, then neither a nor b is 0; or
(b) when M is 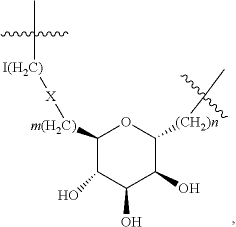
X is O or S, and neither l, m, nor n is 0; or
(c) when M is 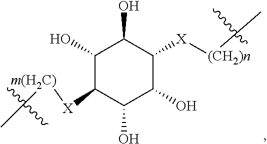
X is O or S, and neither m nor n is 0.

9. The linker according to claim 7, wherein m=n.

10. The linker according to claim 2, wherein Y₁ and Y₂ are independently selected from: —(CH₂)_{m′}(OCH₂CH₂)_{t′}O(CH₂)_{n′}—, —(CH₂)_{m′}O(CH₂)_{n′}O—, —[CH₂]_{m′}(CHQ′)[CH₂]n′O—, —[CH₂]_{m′}(CHQ′)[CH₂]_{n′}S—, 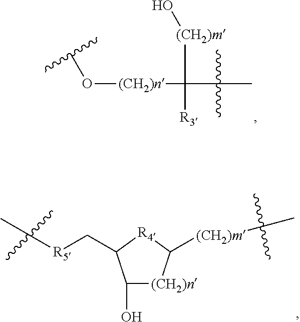
(CH₂)_{m′}NHCO(CH₂)_{n′}—, —(CH₂)_{m′}O—(CH₂)_{n′}—, —(CH₂)_{l′}(OCH₂CH₂)_{n′}O(CH₂)_{m′}CONH—, 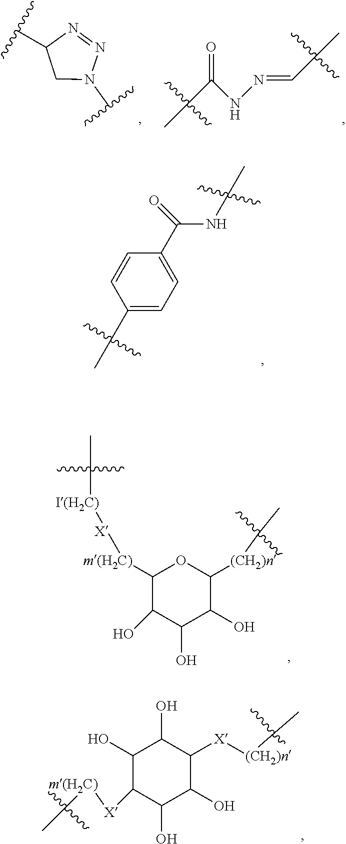
or combinations thereof.

11. The linker according to claim 10, wherein when one end of Y₁ or Y₂ is an oxygen (O) or sulfur (S) atom bonded to other structural fragment beyond Y₁ or Y₂, the O or S atom is bonded through a wavy line shown in any one of formulas A, B, C, E, or F.

12. The linker according to claim 10, wherein Y₁ and Y₂ are the same.

13. The linker according to claim 10, wherein:
(a) when Y₁ or Y₂ is —(CH₂)_{m′}(OCH₂CH₂)_{t′}O(CH₂)_{n′}—, the values of m′, n′, and t′ satisfy any one of the following conditions:
(i) m′=0, t′=0, and n′≠0;
(ii) m′=0, n′=0, and t′≠0;
(iii) m′ 0, t′≠0, and n′=0;
(iv) n′=0, t′=0, and m′≠0;
(v) m′=0, t′≠0, and n′≠0;
(b) when Y₁ or Y₂ is —(CH₂)_{m′}O(CH₂)_{n′}O—, neither m′ nor n′ is 0;
(c) when Y₁ or Y₂ is —[CH₂]_{m″}(CHQ′)[CH₂]_{n′}O—, neither m′ nor n′ is 0, and Q′ is H or —(CH₂)ₐCH₃;
(d) when Y₁ or Y₂ is —[CH₂]_{m′}(CHQ′)[CH₂]_{n′}S—, Q′ is —H, neither m′ nor n′ is 0;
(e) when Y₁ or Y₂ is 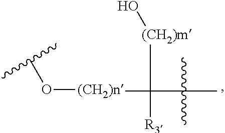
R₃ is —(CH₂)_{x′}CH₃;
(f) when Y₁ or Y₂ is 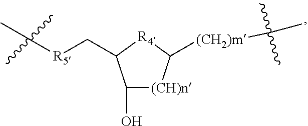
R₄ and R₅ are each —O—, neither m′ nor n′ is 0;
(g) when Y₁ or Y₂ is a combination of groups selected from —(CH₂)_{m′}NHCO(CH₂)_{n′}—, 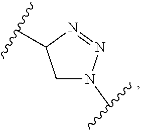 [SMILES: *C1CN(*)N=N1]
(CH₂)_{m′}O—(CH₂)_{n′}—, 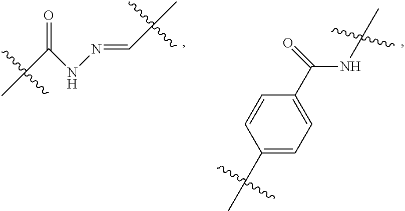 [SMILES: */C=N/NC(*)=O ; *NC(=O)c1ccc(*)cc1]
and —(CH₂)_{l′}(OCH₂CH₂)_{n′}O(CH₂)_{m′}CONH—, the groups are connected in sequence, wherein neither l′, m′, nor n′ is 0;
(h) when Y₁ or Y₂ is 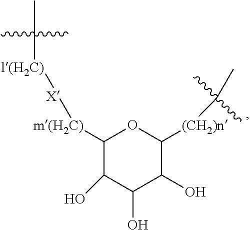
X′ is either O or S, and neither m′ nor n′ is 0;
(i) when Y₁ or Y₂ is 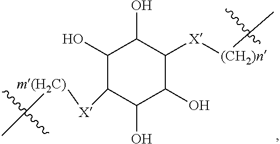
X′ is either O or S, and neither m′ nor n′ is 0;
(j) when Y₁ or Y₂ is 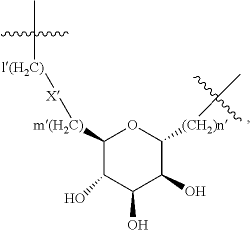
X′ is either O or S, and neither m′ nor n′ is 0;
(k) when Y₁ or Y₂ is 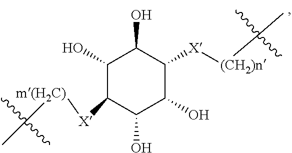
X′ is either O or S, and neither m′ nor n′ is 0.

14. The linker according to claim 2, wherein:
(a) M is a C₁-C₃₀ alkylene group; and Y₁ and Y₂ are selected from —(CH₂)_{m′}(OCH₂CH₂)_{t′}O(CH₂)_{n′}—, —(CH₂)_{m′}O(CH₂)_{n′}O—, —[CH₂]_{m′}(CHQ′)[CH₂]n′O—, —[CH₂]_{m′}(CHQ′)[CH₂]_{n′}S—, 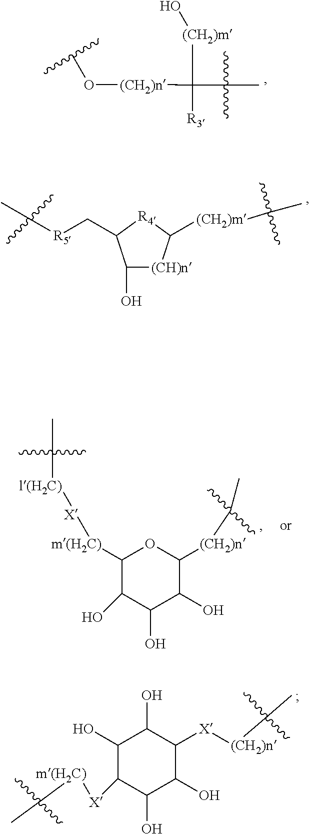
(b) when M is —[CH₂]ₘ(CHQ)[CH₂]ₙ—, Q is —(CH₂)ₐ(OCH₂CH₂)_{b}O(CH₂)_{c}—, wherein a, b, and c are each 0, and neither m nor n is 0; and Y₁ and/or Y₂ is —(CH₂)_{m′}(OCH₂CH₂)_{t′}O(CH₂)_{n′}—, wherein m′ and t′ are each 0 and n′ is a non-zero integer;
(c) when M is —(CH₂)ₘ(CH═CH)ₜ(CH₂)ₙ—, neither m, n, nor t is 0; and Y₁ and/or Y₂ is —(CH₂)_{m′}(OCH₂CH₂)_{t′}O(CH₂)_{n′}—, wherein n′ and t′ are each 0, and m′ is a non-zero integer;
(d) when M is —(CH₂)ₘNQ (CH₂)ₙ—, neither m nor n is 0, Q is selected from —(CH₂)_{d}CH₃, —(CH₂)ₐCO(CH₂)_{b}O—, or —(CH₂)ₐCONH(CH₂)_{b}O—; and Y₁ and/or Y₂ is —(CH₂)_{m′}(OCH₂CH₂)_{t′}O(CH₂)_{n′}—, wherein n′ and t′ are each 0, and m′ is a non-zero integer;
(e) when M is —(CH₂)ₘOP(═O)(X)O(CH₂)ₙ—, neither m nor n is 0, and X is selected from OH, SH, O⁻, or S⁻; and Y₁ and/or Y₂ is —(CH₂)_{m′}(OCH₂CH₂)_{t′}O(CH₂)_{n′}—, wherein n′ and t′ are each 0, and m′ is a non-zero integer;
(f) when M is —(CH₂)ₘOP(═S)(Y)O(CH₂)ₙ—, neither m nor n is 0, and Y is selected from OH, SH, O⁻, or S⁻; and Y₁ and/or Y₂ is —(CH₂)_{m′}(OCH₂CH₂)_{t′}O(CH₂)_{n′}—, wherein n′ and t′ are each 0, and m′ is a non-zero integer;
(g) when M is —(CH₂)ₘ(OCH₂CH₂)ₜO(CH₂)ₙ—, neither m, n, nor t is 0; and Y₁ and/or Y₂ is —(CH₂)_{m′}(OCH₂CH₂)_{t′}O(CH₂)_{n′}—, wherein n′ and t′ are each 0, and m′ is a non-zero integer;
(h) when M is 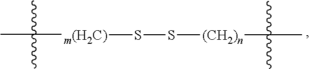
neither m nor n is 0; and Y₁ and/or Y₂ is —(CH₂)_{m′}(OCH₂CH₂)_{t′}O(CH₂)_{n′}—, wherein n′ and t′ are each 0, and m′ is a non-zero integer;
(i) when M is —(CH₂)ₘ—O—SiO—O—(CH₂)ₙ—, neither m nor n is 0; and Y₁ and/or Y₂ is —(CH₂)_{m′}(OCH₂CH₂)_{t′}O(CH₂)_{n′}—, wherein n′ and t′ are each 0, and m′ is a non-zero integer;
(j) when M is 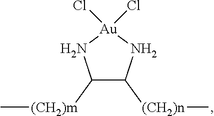
neither m nor n is 0; and Y₁ and/or Y₂ is —(CH₂)_{m′}(OCH₂CH₂)_{t′}O(CH₂)_{n′}—, wherein n′ and t′ are each 0, and m′ is a non-zero integer;
(k) when M is 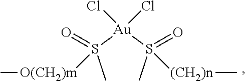
neither m nor n is 0; and Y₁ and/or Y₂ is —(CH₂)_{m′}(OCH₂CH₂)_{t′}O(CH₂)_{n′}—, wherein n′ and t′ are each 0, and m′ is a non-zero integer;
(l) when M is 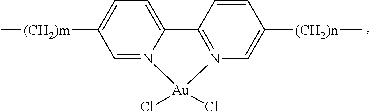
neither m nor n is 0; and Y₁ and/or Y₂ is —(CH₂)_{m′}(OCH₂CH₂)·O(CH₂)_{n′}—, wherein n′ and t′ are each 0, and m′ is a non-zero integer;
(m) when M is 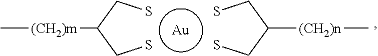
neither m nor n is 0; and Y₁ and/or Y₂ is —(CH₂)_{m′}(OCH₂CH₂)_{t′}O(CH₂)_{n′}—, wherein n′ and t′ are each 0, and m′ is a non-zero integer;
(n) when M is 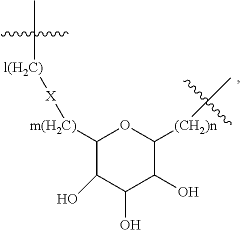
X is O or S, neither m nor n is 0; and Y₁ and/or Y₂ is —(CH₂)_{m′}(OCH₂CH₂)_{t′}O(CH₂)_{n′}—, wherein neither n′ nor t′ is 0, and m′ is 0;
(o) when M is 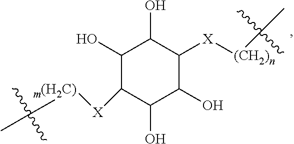
neither m nor n is 0; and Y₁ and/or Y₂ is —(CH₂)_{m′}(OCH₂CH₂)_{t′}O(CH₂)_{n′}—, wherein n′ and t′ are each 0, and m′ is a non-zero integer;
(p) when M is —(CH₂)ₙOP(═O)OHO—, n is 0; and Y₁ and/or Y₂ is —(CH₂)_{m′}(OCH₂CH₂)_{t′}O(CH₂)_{n′}—, wherein n′ is 0 and neither m′ nor t is 0;
(q) when M is 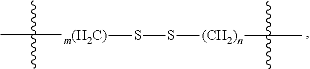
neither m nor n is 0; and Y₁ and/or Y₂ is —(CH₂)_{m′}(OCH₂CH₂)_{t′}O(CH₂)_{n′}—, wherein t′ is a non-zero integer, and n′ and m′ are each 0;
(r) when M is 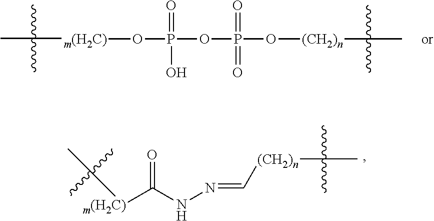
neither m nor n is 0; and Y₁ and/or Y₂ is —(CH₂)_{m′}(OCH₂CH₂)_{t′}O(CH₂)_{n′}—, wherein n′ and t′ are each 0, and m′ is a non-zero integer; or Y₁ and/or Y₂ is —(CH₂)_{m′}(OCH₂CH₂)_{t′}O(CH₂)_{n′}—, wherein m′ is 0, n′ and t′ are each a non-zero integer;
(s) M is a C₁-C₃₀ alkylene group; and Y₁ and/or Y₂ is a combination of groups selected from: —(CH₂)_{m′}NHCO(CH₂)_{n′}—, 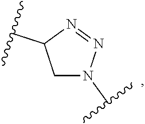 [SMILES: *C1CN(*)N=N1]
—(CH₂)_{m′}O—(CH₂)_{n′}—, 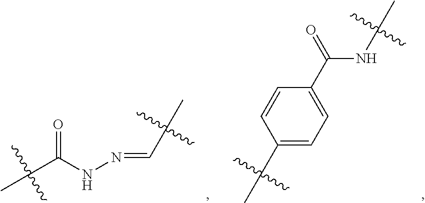 [SMILES: */C=N/NC(*)=O ; *NC(=O)c1ccc(*)cc1]
and —(CH₂)_{l′}(OCH₂CH₂)_{n′}O(CH₂)_{m′}CONH—, or is 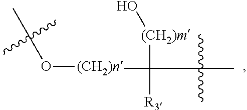
wherein the groups in the combination are connected in sequence, and neither l′, m′ nor n′ is 0;

15. The linker according to claim 14, wherein any of l′, n′, m′, t′, x′, a′, b′, c′, l, n, m, t, x, a, b, c, d, or e is independently selected from an integer falling within any one of the following ranges: 0-15, 0-10, 0-8, 0-7, 0-6, 0-5, 0-4, 0-3, 0-2, 1-15, 1-10, 1-8, 1-7, 1-6, 1-5, 1-3, 1-2.

16. The linker according to claim 2, wherein comprises a structure shown below:

| No. | Structure |
|---|---|
| A1 | 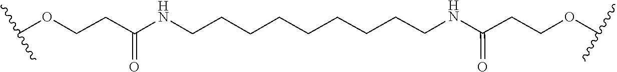 [SMILES: *OCCC(=O)NCCCCCCCCCNC(=O)CCO*] |
| A2 | 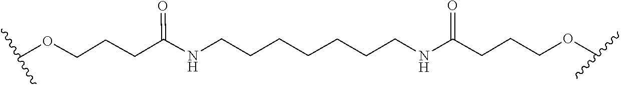 [SMILES: *OCCCC(=O)NCCCCCCCNC(=O)CCCO*] |
| A3 | 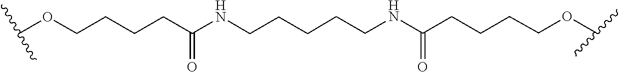 [SMILES: *OCCCCC(=O)NCCCCCNC(=O)CCCCO*] |
| A4 | 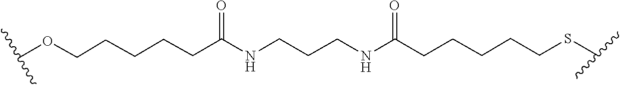 [SMILES: *OCCCCCC(=O)NCCCNC(=O)CCCCCS*] |
| A5 | 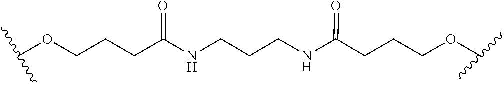 [SMILES: *OCCCC(=O)NCCCNC(=O)CCCO*] |
| A6 | 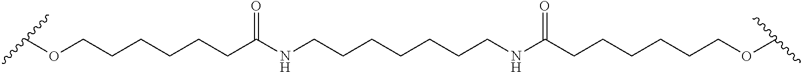 [SMILES: *OCCCCCCC(=O)NCCCCCCCNC(=O)CCCCCCO*] |
| A7 | 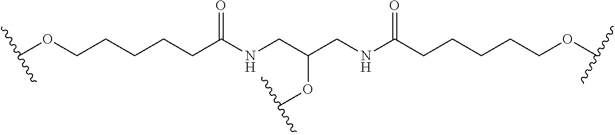 [SMILES: *OCCCCCC(=O)NCC(CNC(=O)CCCCCO*)O*] |
| A8 | 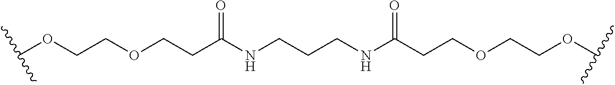 [SMILES: *OCCOCCC(=O)NCCCNC(=O)CCOCCO*] |
| A9 | 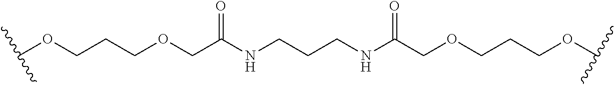 [SMILES: *OCCCOCC(=O)NCCCNC(=O)COCCCO*] |
| A10 | 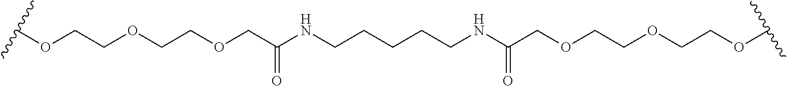 [SMILES: *OCCOCCOCC(=O)NCCCCCNC(=O)COCCOCCO*] |
| A11 | 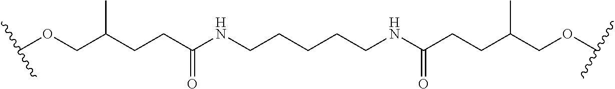 [SMILES: *OCC(C)CCC(=O)NCCCCCNC(=O)CCC(C)CO*] |
| A12 | 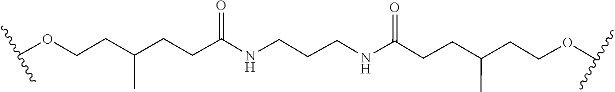 [SMILES: *OCCC(C)CCC(=O)NCCCNC(=O)CCC(C)CCO*] |
| A13 | 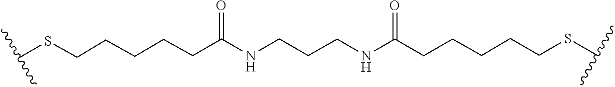 [SMILES: *SCCCCCC(=O)NCCCNC(=O)CCCCCS*] |
| A14 | 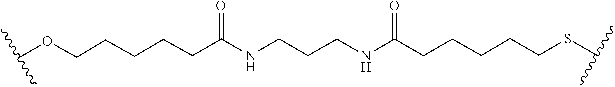 [SMILES: *OCCCCCC(=O)NCCCNC(=O)CCCCCS*] |
| A15 | 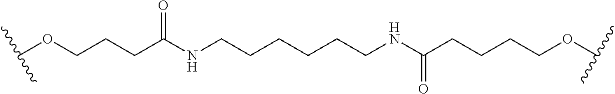 [SMILES: *OCCCCC(=O)NCCCCCCNC(=O)CCCO*] |
| A16 | 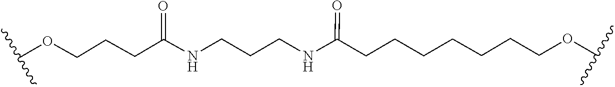 [SMILES: *OCCCCCCCC(=O)NCCCNC(=O)CCCO*] |
| A17 | 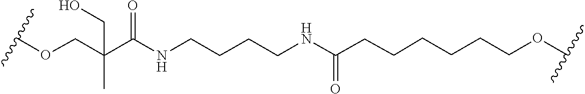 [SMILES: *OCCCCCCC(=O)NCCCCNC(=O)C(C)(CO)CO*] |
| A18 | 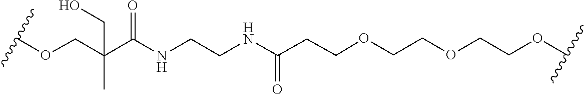 [SMILES: *OCCOCCOCCC(=O)NCCNC(=O)C(C)(CO)CO*] |
| A19 | 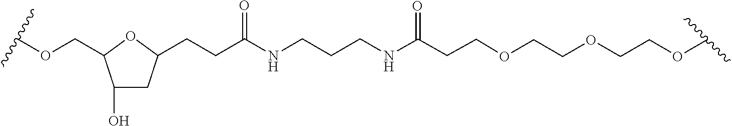 [SMILES: *OCCOCCOCCC(=O)NCCCNC(=O)CCC1CC(O)C(CO*)O1] |
| A20 | 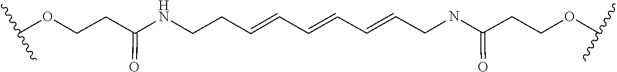 |
| A21 | 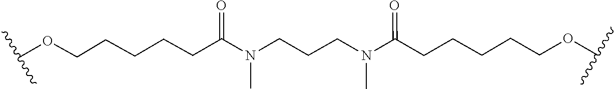 [SMILES: *OCCCCCC(=O)N(C)CCCN(C)C(=O)CCCCCO*] |
| A22 | 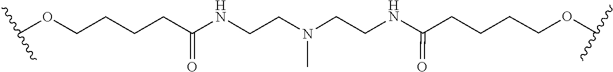 [SMILES: *OCCCCC(=O)NCCN(C)CCNC(=O)CCCCO*] |
| A23 | 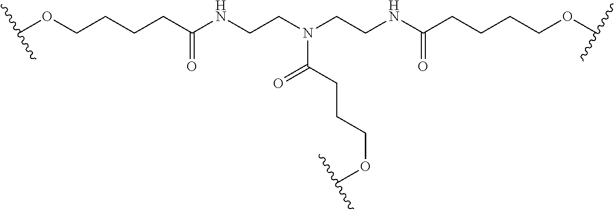 [SMILES: *OCCCCC(=O)NCCN(CCNC(=O)CCCCO*)C(=O)CCCO*] |
| A24 | 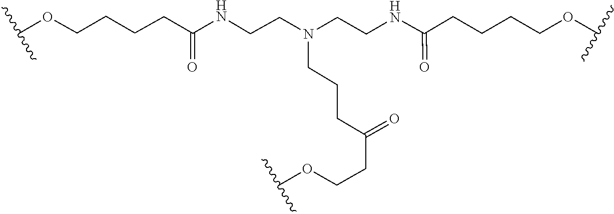 [SMILES: *OCCCCC(=O)NCCN(CCCC(=O)CCO*)CCNC(=O)CCCCO*] |
| A25 | 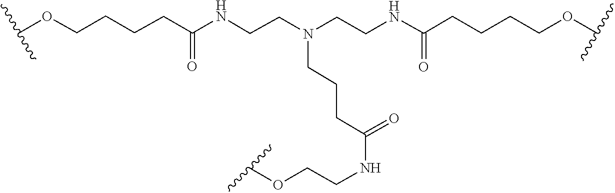 [SMILES: *OCCCCC(=O)NCCN(CCCC(=O)NCCO*)CCNC(=O)CCCCO*] |
| A26 | 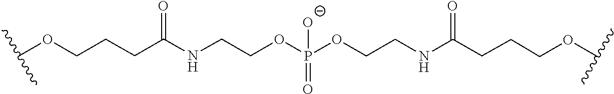 [SMILES: *OCCCC(=O)NCCOP(=O)([O-])OCCNC(=O)CCCO*] |
| A27 | 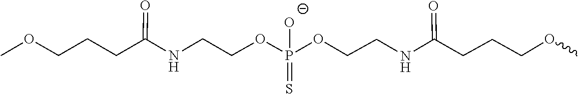 |
| A28 | 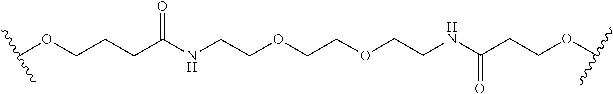 [SMILES: *OCCCC(=O)NCCOCCOCCNC(=O)CCO*] |
| A29 | 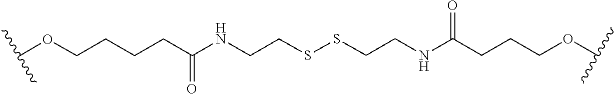 [SMILES: *OCCCCC(=O)NCCSSCCNC(=O)CCCO*] |
| A30 | 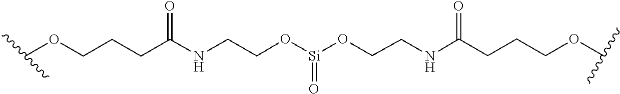 [SMILES: *OCCCC(=O)NCCO[Si](=O)OCCNC(=O)CCCO*] |
| A31 | 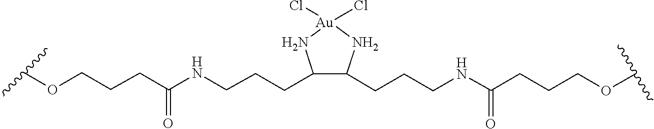 |
| A32 | 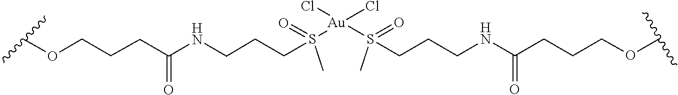 |
| A33 | 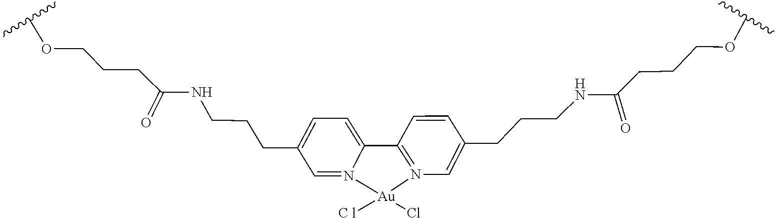 |
| A34 | 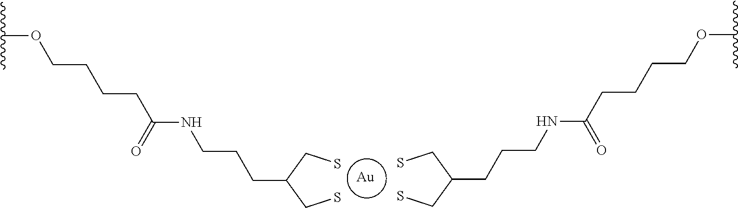 |
| A35 | 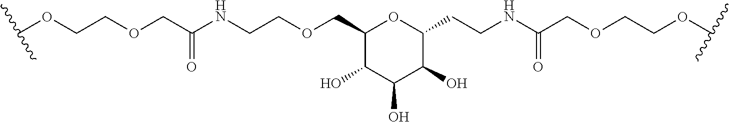 [SMILES: *OCCOCC(=O)NCCOC[C@H]1O[C@H](CCNC(=O)COCCO*)[C@@H](O)[C@@H](O)[C@@H]1O] |
| A36 | 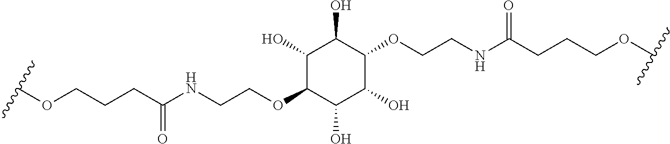 [SMILES: *OCCCC(=O)NCCO[C@H]1[C@H](O)[C@@H](O)[C@H](OCCNC(=O)CCCO*)[C@@H](O)[C@H]1O] |
| A37 | 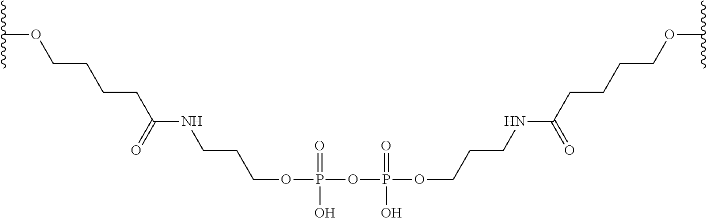 [SMILES: *OCCCCC(=O)NCCCOP(=O)(O)OP(=O)(O)OCCCNC(=O)CCCCO*] |
| A38 | 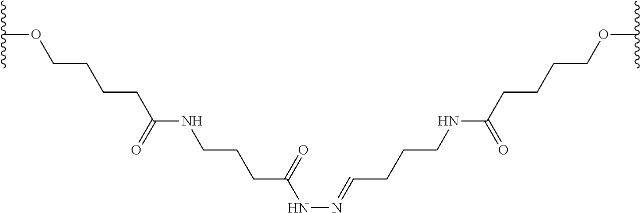 [SMILES: *OCCCCC(=O)NCCC/C=N/NC(=O)CCCNC(=O)CCCCO*] |
| No. | Structure |
|---|---|
| B1 | 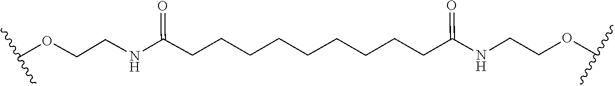 [SMILES: *OCCNC(=O)CCCCCCCCCC(=O)NCCO*] |
| B2 | 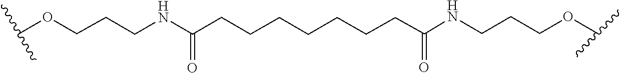 [SMILES: *OCCCNC(=O)CCCCCCCC(=O)NCCCO*] |
| B3 | 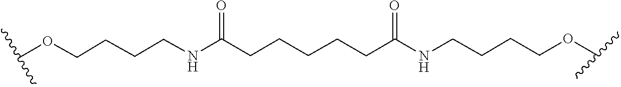 [SMILES: *OCCCCNC(=O)CCCCCC(=O)NCCCCO*] |
| B4 | 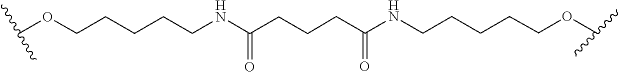 [SMILES: *OCCCCCNC(=O)CCCC(=O)NCCCCCO*] |
| B5 | 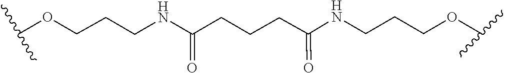 [SMILES: *OCCCNC(=O)CCCC(=O)NCCCO*] |
| B6 | 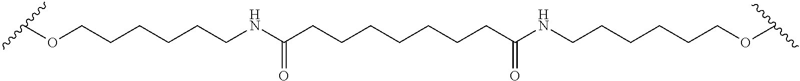 [SMILES: *OCCCCCCNC(=O)CCCCCCCC(=O)NCCCCCCO*] |
| B7 | 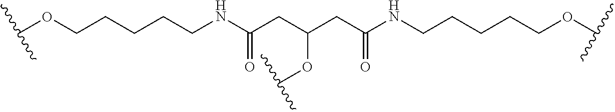 [SMILES: *OCCCCCNC(=O)CC(CC(=O)NCCCCCO*)O*] |
| B8 | 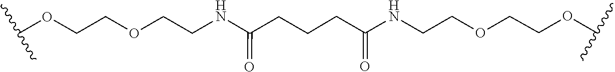 [SMILES: *OCCOCCNC(=O)CCCC(=O)NCCOCCO*] |
| B9 | 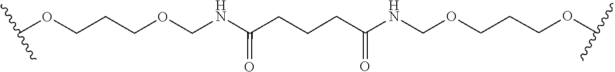 [SMILES: *OCCCOCNC(=O)CCCC(=O)NCOCCCO*] |
| B10 | 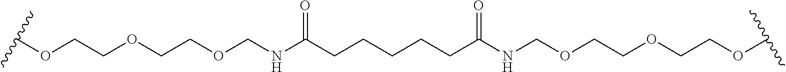 [SMILES: *OCCOCCOCNC(=O)CCCCCC(=O)NCOCCOCCO*] |
| B11 | 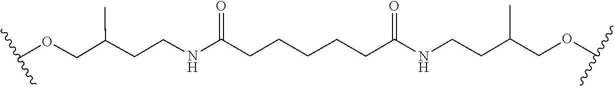 [SMILES: *OCC(C)CCNC(=O)CCCCCC(=O)NCCC(C)CO*] |
| B12 | 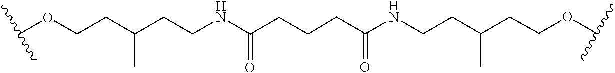 [SMILES: *OCCC(C)CCNC(=O)CCCC(=O)NCCC(C)CCO*] |
| B13 | 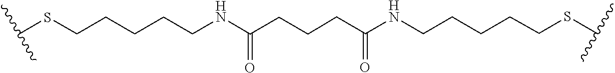 [SMILES: *SCCCCCNC(=O)CCCC(=O)NCCCCCS*] |
| B14 | 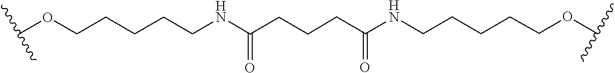 [SMILES: *OCCCCCNC(=O)CCCC(=O)NCCCCCO*] |
| B15 | 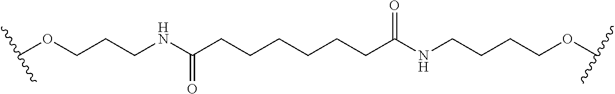 [SMILES: *OCCCCNC(=O)CCCCCCC(=O)NCCCO*] |
| B16 | 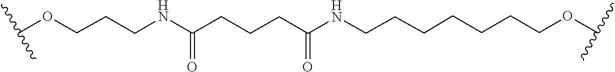 [SMILES: *OCCCCCCCNC(=O)CCCC(=O)NCCCO*] |
| B17 | 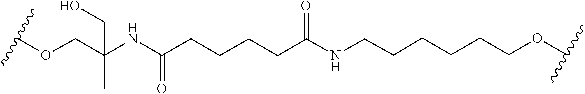 [SMILES: *OCCCCCCNC(=O)CCCCC(=O)NC(C)(CO)CO*] |
| B18 | 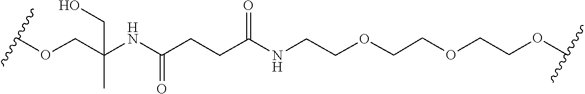 [SMILES: *OCCOCCOCCNC(=O)CCC(=O)NC(C)(CO)CO*] |
| B19 | 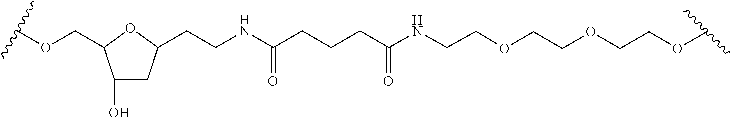 [SMILES: *OCCOCCOCCNC(=O)CCCC(=O)NCCC1CC(O)C(CO*)O1] |
| B20 | 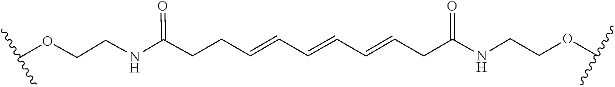 [SMILES: *OCCNC(=O)C/C=C/C=C/C=C/CCC(=O)NCCO*] |
| B21 | 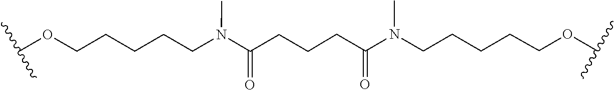 [SMILES: *OCCCCCN(C)C(=O)CCCC(=O)N(C)CCCCCO*] |
| B22 | 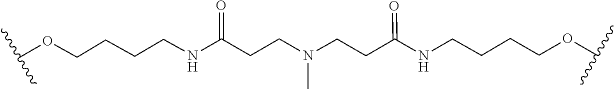 [SMILES: *OCCCCNC(=O)CCN(C)CCC(=O)NCCCCO*] |
| B23 | 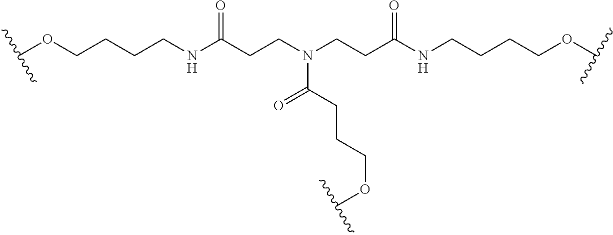 [SMILES: *OCCCCNC(=O)CCN(CCC(=O)NCCCCO*)C(=O)CCCO*] |
| B24 | 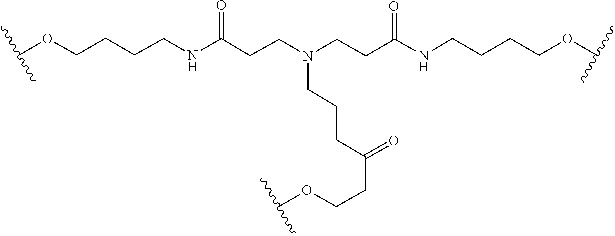 [SMILES: *OCCCCNC(=O)CCN(CCCC(=O)CCO*)CCC(=O)NCCCCO*] |
| B25 | 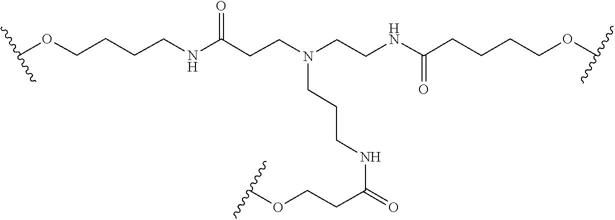 [SMILES: *OCCCCNC(=O)CCN(CCCNC(=O)CCO*)CCNC(=O)CCCCO*] |
| B26 | 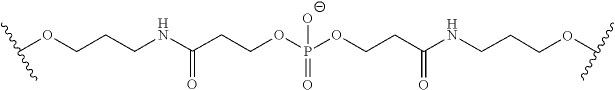 [SMILES: *OCCCNC(=O)CCOP(=O)([O-])OCCC(=O)NCCCO*] |
| B27 | 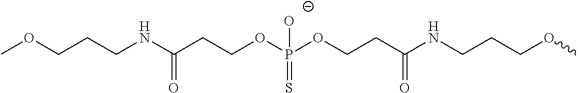 |
| B28 | 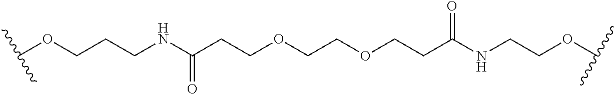 [SMILES: *OCCCNC(=O)CCOCCOCCC(=O)NCCO*] |
| B29 | 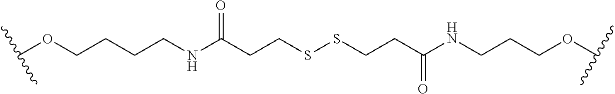 [SMILES: *OCCCCNC(=O)CCSSCCC(=O)NCCCO*] |
| B30 | 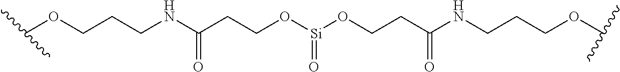 |
| B31 | 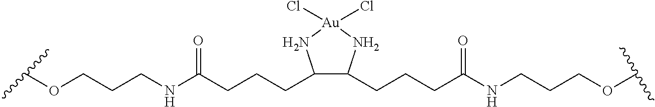 |
| B32 | 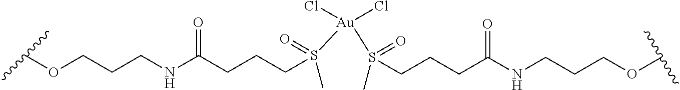 |
| B33 | 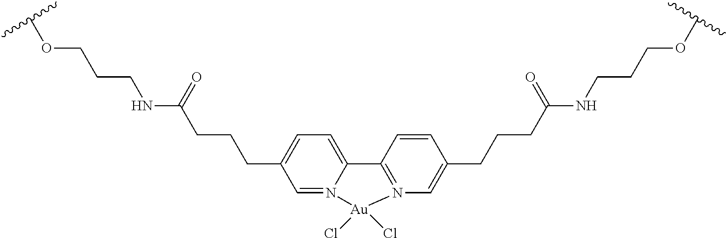 |
| B34 | 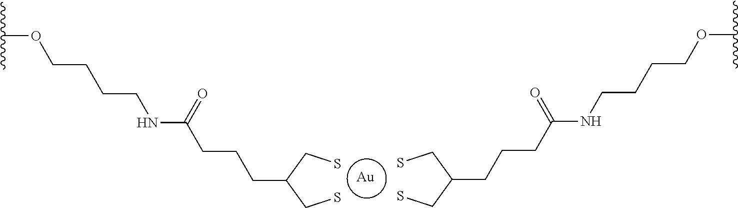 |
| B35 | 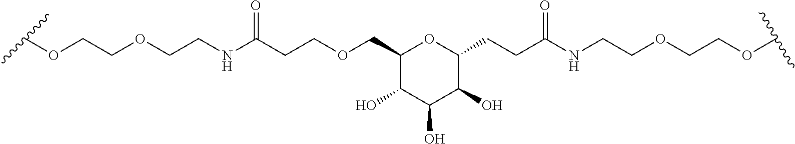 [SMILES: *OCCOCCNC(=O)CCOC[C@H]1O[C@H](CCC(=O)NCCOCCO*)[C@@H](O)[C@@H](O)[C@@H]1O] |
| B36 | 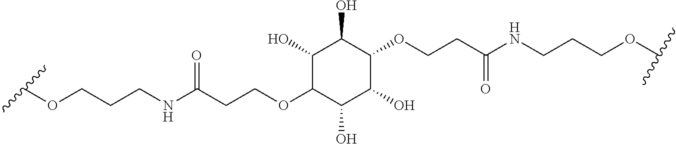 [SMILES: *OCCCNC(=O)CCOC1[C@@H](O)[C@@H](O)[C@@H](OCCC(=O)NCCCO*)[C@H](O)[C@H]1O] |
| B37 | 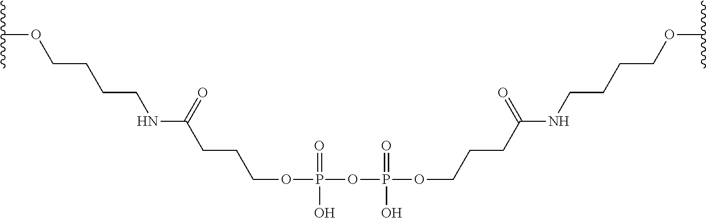 [SMILES: *OCCCCNC(=O)CCCOP(=O)(O)OP(=O)(O)OCCCC(=O)NCCCCO*] |
| B38 | 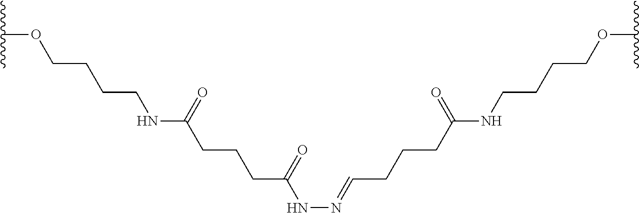 [SMILES: *OCCCCNC(=O)CCC/C=N/NC(=O)CCCC(=O)NCCCCO*] |
| No. | Structure |
|---|---|
| C1 | 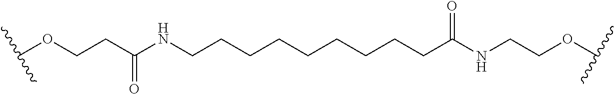 [SMILES: *OCCNC(=O)CCCCCCCCCNC(=O)CCO*] |
| C2 | 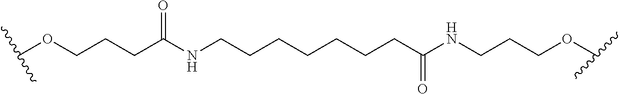 [SMILES: *OCCCNC(=O)CCCCCCCNC(=O)CCCO*] |
| C3 | 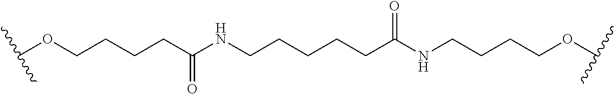 [SMILES: *OCCCCNC(=O)CCCCCNC(=O)CCCCO*] |
| C4 | 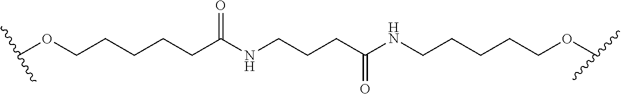 [SMILES: *OCCCCCNC(=O)CCCNC(=O)CCCCCO*] |
| C5 | 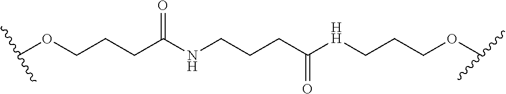 |
| C6 | 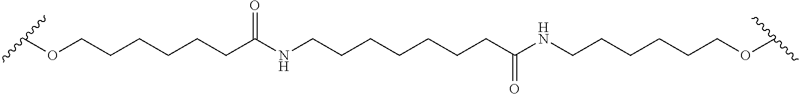 [SMILES: *OCCCCCCNC(=O)CCCCCCCNC(=O)CCCCCCO*] |
| C7 | 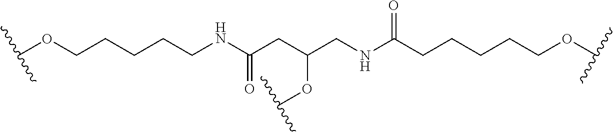 [SMILES: *OCCCCCNC(=O)CC(CNC(=O)CCCCCO*)O*] |
| C8 | 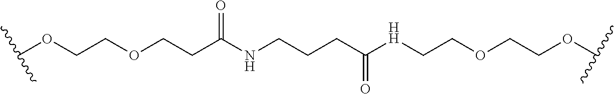 |
| C9 | 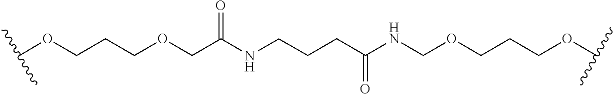 [SMILES: *OCCCOCNC(=O)CCCNC(=O)COCCCO*] |
| C10 | 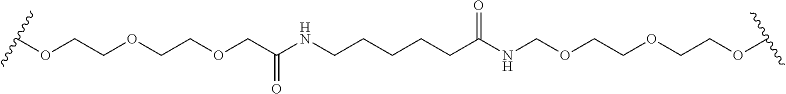 [SMILES: *OCCOCCOCNC(=O)CCCCCNC(=O)COCCOCCO*] |
| C11 | 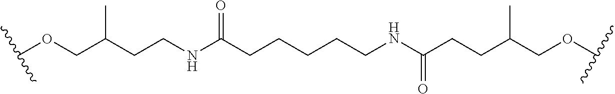 [SMILES: *OCC(C)CCNC(=O)CCCCCNC(=O)CCC(C)CO*] |
| C12 | 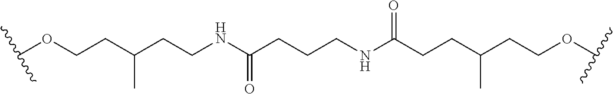 [SMILES: *OCCC(C)CCNC(=O)CCCNC(=O)CCC(C)CCO*] |
| C13 | 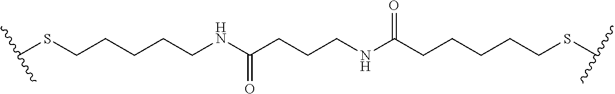 [SMILES: *SCCCCCNC(=O)CCCNC(=O)CCCCCS*] |
| C14 | 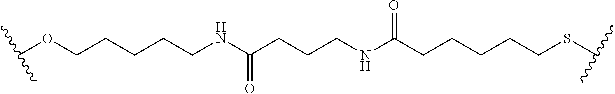 [SMILES: *OCCCCCNC(=O)CCCNC(=O)CCCCCS*] |
| C15 | 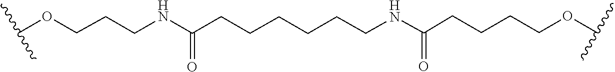 [SMILES: *OCCCCC(=O)NCCCCCCC(=O)NCCCO*] |
| C16 | 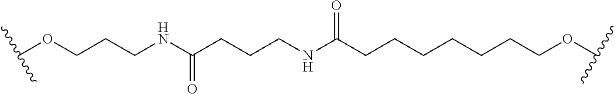 [SMILES: *OCCCCCCCC(=O)NCCCC(=O)NCCCO*] |
| C17 | 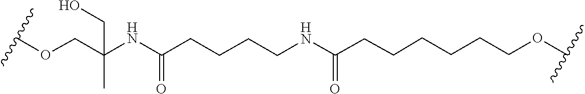 [SMILES: *OCCCCCCC(=O)NCCCCC(=O)NC(C)(CO)CO*] |
| C18 | 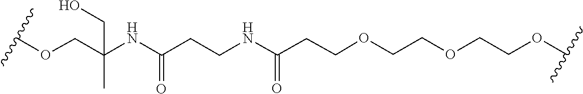 [SMILES: *OCCOCCOCCC(=O)NCCC(=O)NC(C)(CO)CO*] |
| C19 | 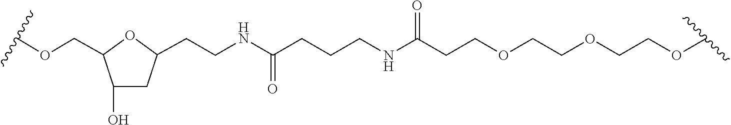 [SMILES: *OCCOCCOCCC(=O)NCCCC(=O)NCCC1CC(O)C(CO*)O1] |
| C20 | 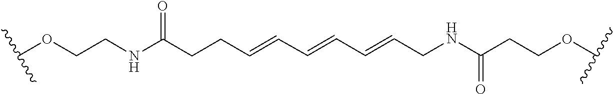 [SMILES: *OCCNC(=O)CC/C=C/C=C/C=C/CNC(=O)CCO*] |
| C21 | 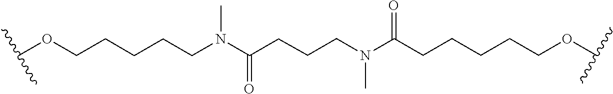 [SMILES: *OCCCCCC(=O)N(C)CCCC(=O)N(C)CCCCCO*] |
| C22 | 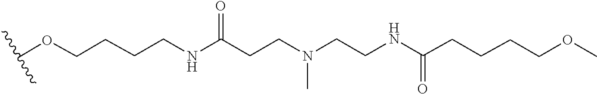 [SMILES: *OCCCCNC(=O)CCN(C)CCNC(=O)CCCCOC] |
| C23 | 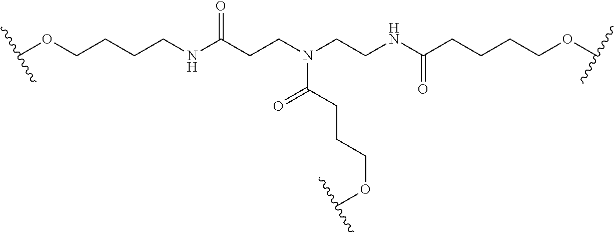 [SMILES: *OCCCCNC(=O)CCN(CCNC(=O)CCCCO*)C(=O)CCCO*] |
| C24 | 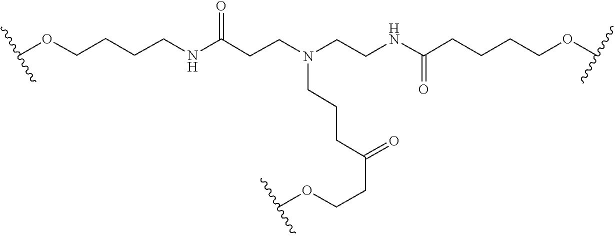 [SMILES: *OCCCCNC(=O)CCN(CCCC(=O)CCO*)CCNC(=O)CCCCO*] |
| C25 | 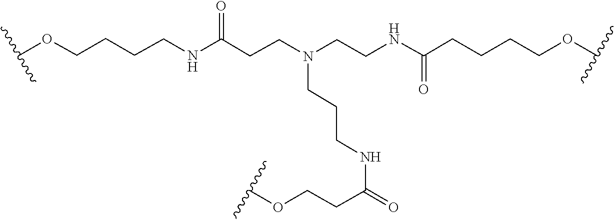 [SMILES: *OCCCCNC(=O)CCN(CCCNC(=O)CCO*)CCNC(=O)CCCCO*] |
| C26 | 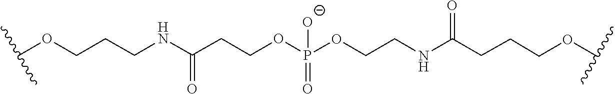 [SMILES: *OCCCNC(=O)CCOP(=O)([O-])OCCNC(=O)CCCO*] |
| C27 | 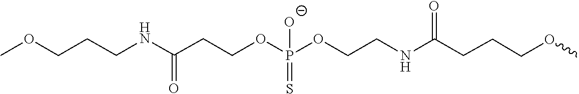 |
| C28 | 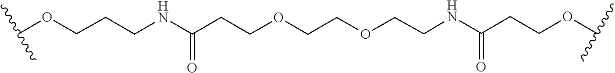 [SMILES: *OCCCNC(=O)CCOCCOCCNC(=O)CCO*] |
| C29 | 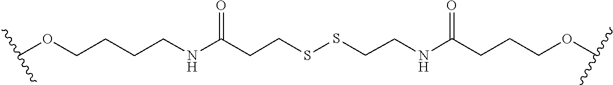 [SMILES: *OCCCCNC(=O)CCSSCCNC(=O)CCCO*] |
| C30 | 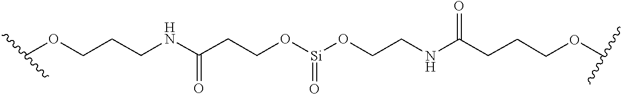 [SMILES: *OCCCNC(=O)CCO[Si](=O)OCCNC(=O)CCCO*] |
| C31 | 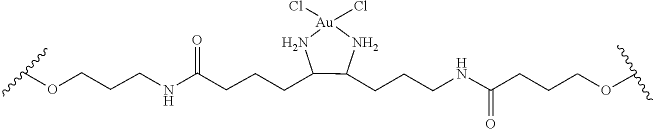 |
| C32 | 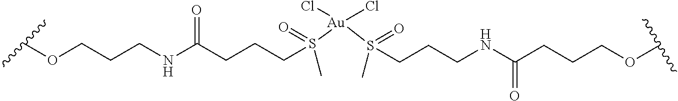 |
| C33 | 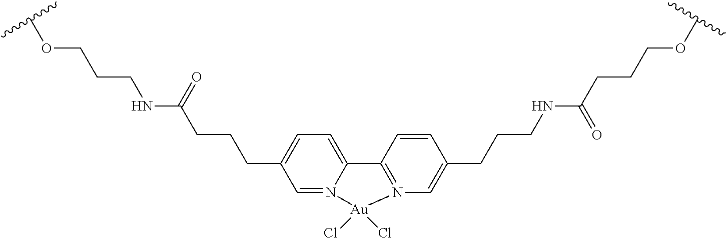 |
| C34 | 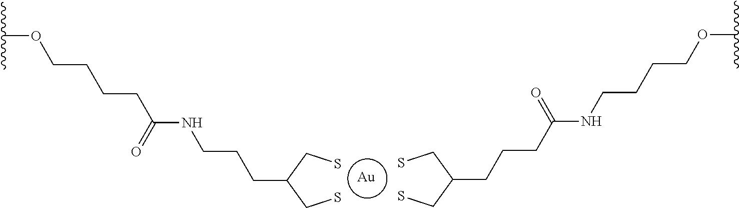 |
| C35 | 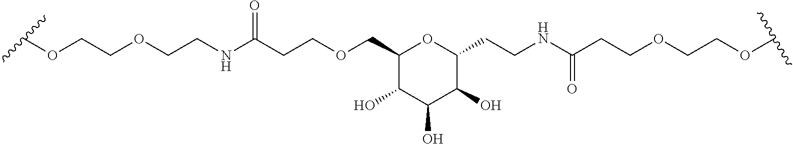 [SMILES: *OCCOCCNC(=O)CCOC[C@H]1O[C@H](CCNC(=O)CCOCCO*)[C@@H](O)[C@@H](O)[C@@H]1O] |
| C36 | 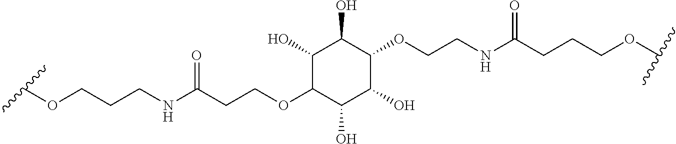 [SMILES: *OCCCNC(=O)CCOC1[C@@H](O)[C@@H](O)[C@@H](OCCNC(=O)CCCO*)[C@H](O)[C@H]1O] |
| C37 | 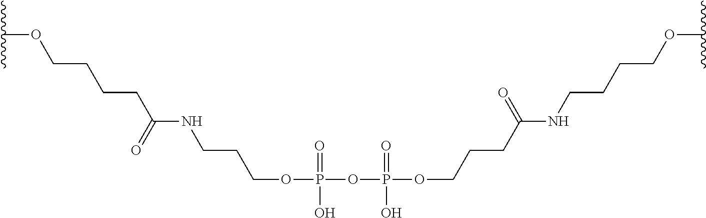 [SMILES: *OCCCCNC(=O)CCCOP(=O)(O)OP(=O)(O)OCCCNC(=O)CCCCO*] |
| C38 | 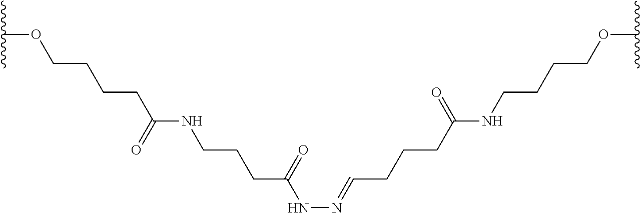 [SMILES: *OCCCCNC(=O)CCC/C=N/NC(=O)CCCNC(=O)CCCCO*] |
| No. | Structure |
|---|---|
| D1 | 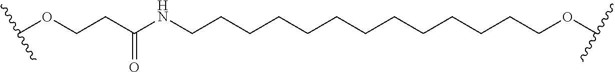 [SMILES: *OCCCCCCCCCCCCCNC(=O)CCO*] |
| D2 | 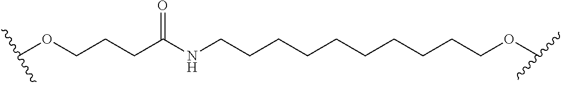 [SMILES: *OCCCCCCCCCCNC(=O)CCCO*] |
| D3 | 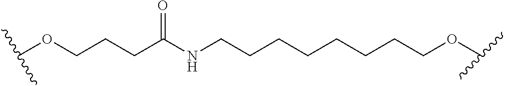 [SMILES: *OCCCCCCCCNC(=O)CCCO*] |
| D4 | 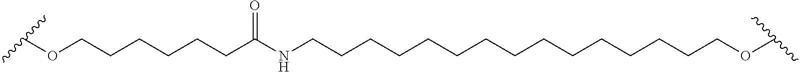 [SMILES: *OCCCCCCCCCCCCCCCNC(=O)CCCCCCO*] |
| D5 | 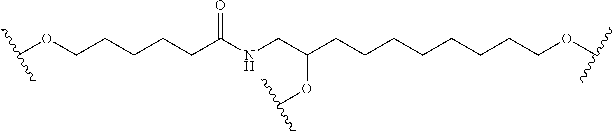 [SMILES: *OCCCCCCCCC(CNC(=O)CCCCCO*)O*] |
| D6 | 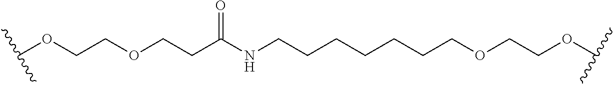 [SMILES: *OCCOCCCCCCCNC(=O)CCOCCO*] |
| D7 | 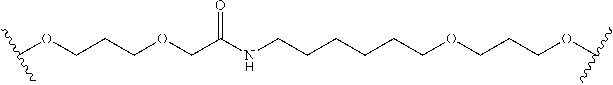 [SMILES: *OCCCOCCCCCCNC(=O)COCCCO*] |
| D8 | 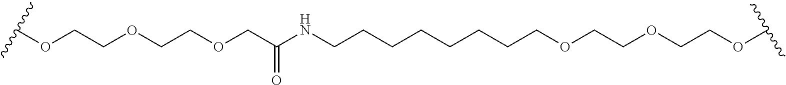 [SMILES: *OCCOCCOCCCCCCCCNC(=O)COCCOCCO*] |
| D9 | 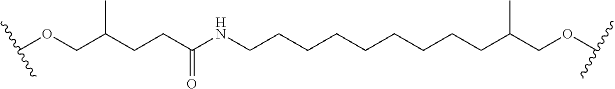 [SMILES: *OCC(C)CCCCCCCCCNC(=O)CCC(C)CO*] |
| D10 | 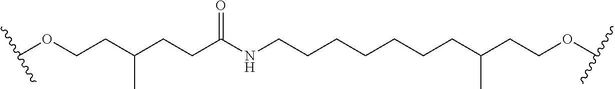 [SMILES: *OCCC(C)CCCCCCCNC(=O)CCC(C)CCO*] |
| D11 | 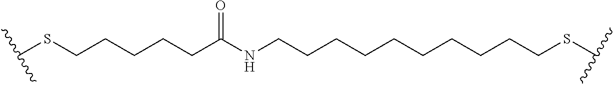 [SMILES: *SCCCCCCCCCCNC(=O)CCCCCS*] |
| D12 | 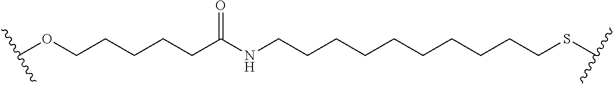 [SMILES: *OCCCCCC(=O)NCCCCCCCCCCS*] |
| D13 | 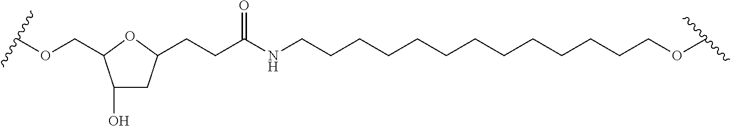 [SMILES: *OCCCCCCCCCCCCCNC(=O)CCC1CC(O)C(CO*)O1] |
| D14 | 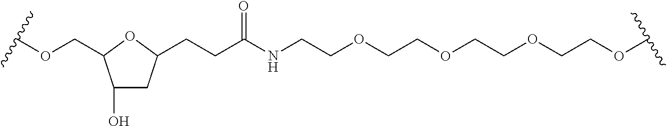 [SMILES: *OCCOCCOCCOCCNC(=O)CCC1CC(O)C(CO*)O1] |
| D15 | 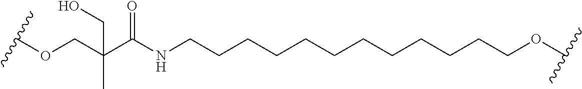 [SMILES: *OCCCCCCCCCCCCNC(=O)C(C)(CO)CO*] |
| D16 | 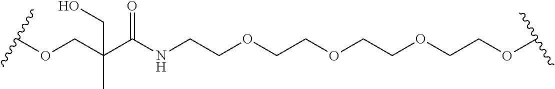 [SMILES: *OCCOCCOCCOCCNC(=O)C(C)(CO)CO*] |
| D17 | 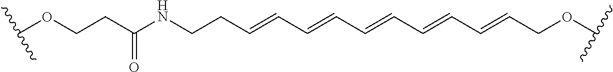 [SMILES: *OC/C=C/C=C/C=C/C=C/C=C/CCNC(=O)CCO*] |
| D18 | 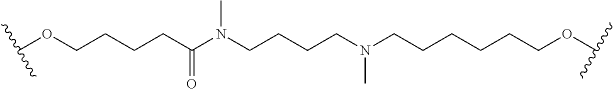 [SMILES: *OCCCCCCN(C)CCCCN(C)C(=O)CCCCO*] |
| D19 | 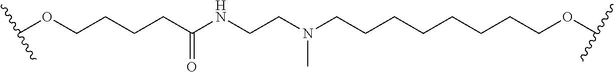 [SMILES: *OCCCCCCCCN(C)CCNC(=O)CCCCO*] |
| D20 | 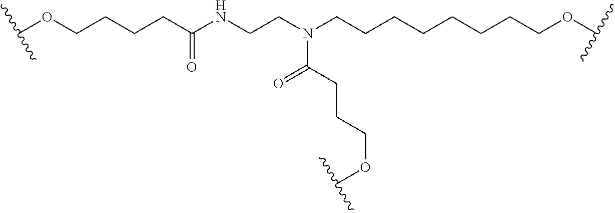 [SMILES: *OCCCCCCCCN(CCNC(=O)CCCCO*)C(=O)CCCO*] |
| D21 | 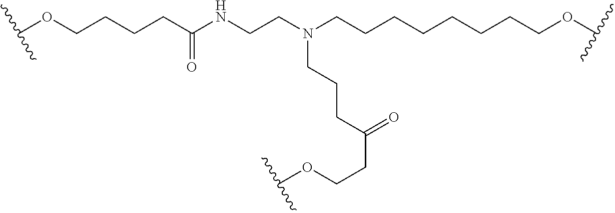 [SMILES: *OCCCCCCCCN(CCCC(=O)CCO*)CCNC(=O)CCCCO*] |
| D22 | 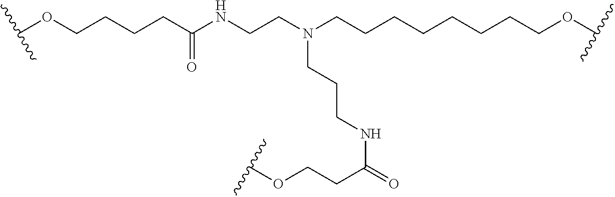 [SMILES: *OCCCCCCCCN(CCCNC(=O)CCO*)CCNC(=O)CCCCO*] |
| D23 | 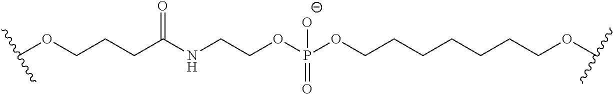 [SMILES: *OCCCCCCCOP(=O)([O-])OCCNC(=O)CCCO*] |
| D24 | 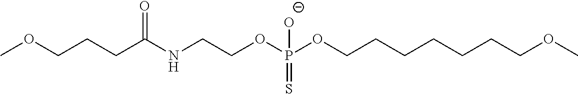 |
| D25 | 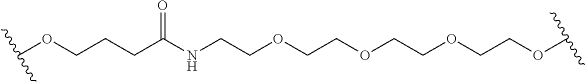 [SMILES: *OCCCC(=O)NCCOCCOCCOCCO*] |
| D26 | 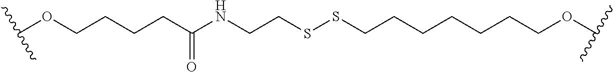 [SMILES: *OCCCCCCCSSCCNC(=O)CCCCO*] |
| D27 | 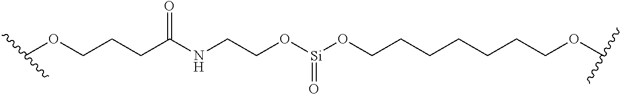 [SMILES: *OCCCCCCCO[Si](=O)OCCNC(=O)CCCO*] |
| D28 | 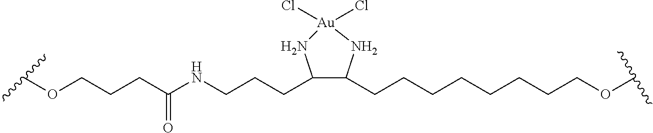 |
| D29 | 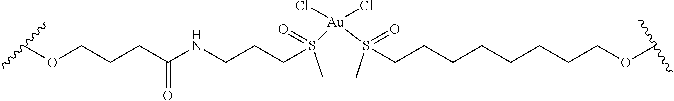 |
| D30 | 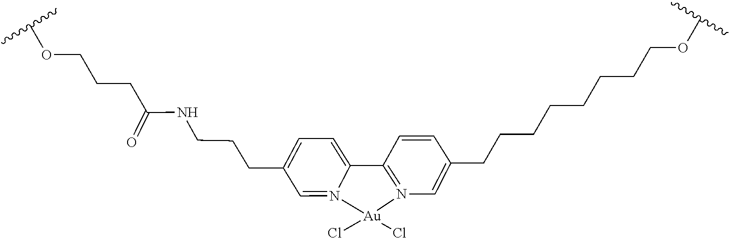 |
| D31 | 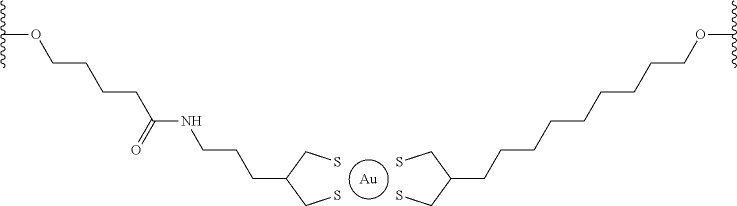 |
| D32 | 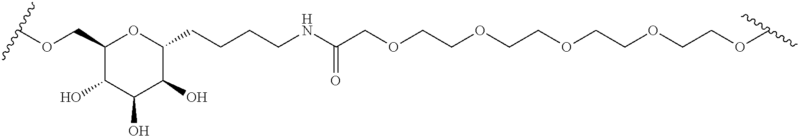 [SMILES: *OCCOCCOCCOCCOCC(=O)NCCCC[C@H]1O[C@H](CO*)[C@@H](O)[C@H](O)[C@@H]1O] |
| D33 | 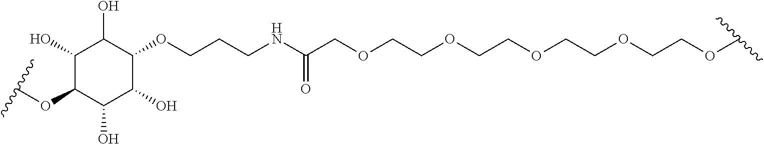 [SMILES: *OCCOCCOCCOCCOCC(=O)NCCCO[C@H]1C(O)[C@@H](O)[C@H](O*)[C@@H](O)[C@H]1O] |
| D34 | 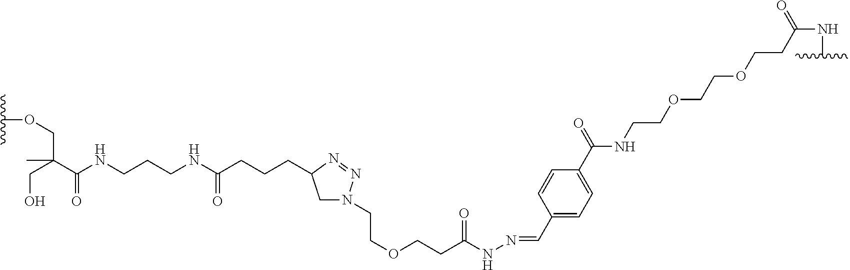 [SMILES: *NC(=O)CCOCCOCCNC(=O)c1ccc(/C=N/NC(=O)CCOCCN2CC(CCCC(=O)NCCCNC(=O)C(C)(CO)CO*)N=N2)cc1] |
| D35 | 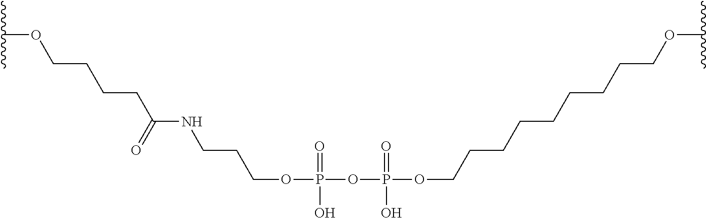 [SMILES: *OCCCCCCCCCOP(=O)(O)OP(=O)(O)OCCCNC(=O)CCCCO*] |
| D36 | 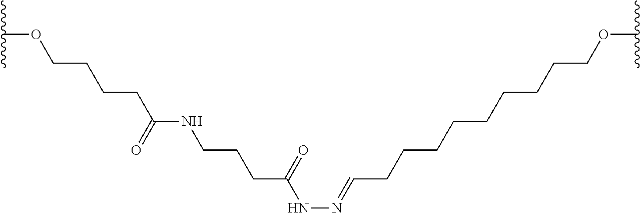 [SMILES: *OCCCCCCCCC/C=N/NC(=O)CCCNC(=O)CCCCO*] |
| No. | Structure |
|---|---|
| E1 | 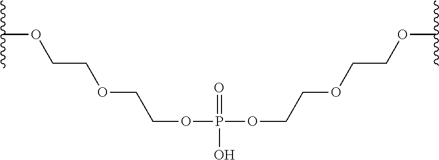 [SMILES: *OCCOCCOP(=O)(O)OCCOCCO*] |
| E2 | 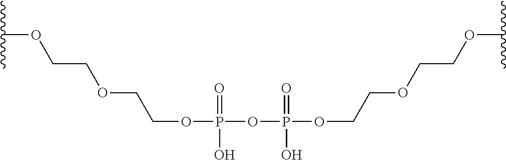 [SMILES: *OCCOCCOP(=O)(O)OP(=O)(O)OCCOCCO*] |
| E3 | 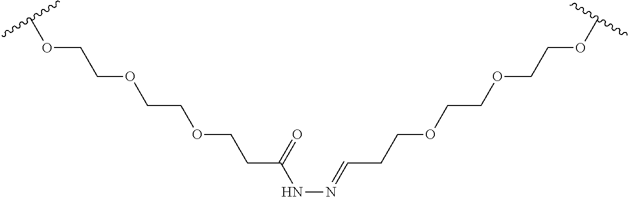 [SMILES: *OCCOCCOCC/C=N/NC(=O)CCOCCOCCO*] |
| E4 | 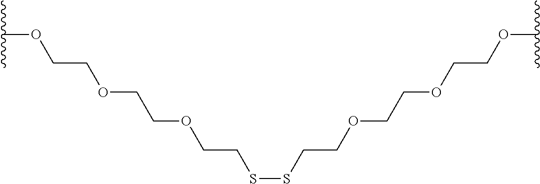 [SMILES: *OCCOCCOCCSSCCOCCOCCO*] |

wherein the wavy lines indicate positions at which the linker is attached to the oligonucleotide and/or the ligand.

17. A compound comprising the linker of claim 2 and two or more oligonucleotides covalently attached to the linker.

18. The compound of claim 17, wherein the two or more oligonucleotides are delivered to a same or a different position of a mRNA or are delivered to two or more different mRNAs.

19. The compound of claim 17, wherein:
the linker is attached to a sense strand or an antisense strand of the two or more oligonucleotides; and/or
the linker is attached to a 3′ end and/or a 5′ end of the two or more oligonucleotides.

20. The compound of claim 17, wherein:
at least one of the two or more oligonucleotides comprises at least two phosphorothioate linkages within the five nucleotides at the terminus proximal to the linker; and/or
at least one of the two or more oligonucleotides comprises a Ral group at the terminus distal to the linker.

21. The compound of claim 17, further comprising a ligand covalently attached to the linker and/or one of the two or more oligonucleotides.

22. The compound according to claim 21, wherein:
the ligand is attached to a sense strand or an antisense strand of the two or more oligonucleotides, and/or
the ligand is attached to a 3′ end and/or a 5′ end of the two or more oligonucleotides.

23. A method of preparing a compound according to claim 21, comprising:
(a) reacting a compound represented by Formula 1 with a compound represented by Formula 2;
(b) reacting a compound represented by Formula 1 with a compound represented by Formula 3; or
(c) reacting a compound represented by Formula 8 with a compound represented by Formula 11;

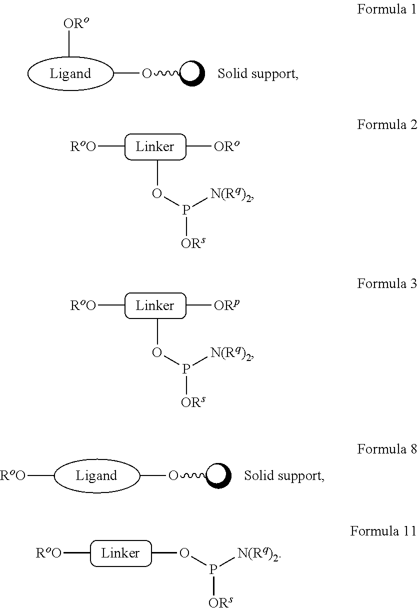

24. The method according to claim 23, further comprising:
reacting one or more of resulting intermediates with RNA phosphoramidites using a solid-phase phosphoramidite monomer chemical synthesis method.

25. The method according to claim 24, comprising:
(a) reacting the compound of Formula 1 with the compound of Formula 2 to produce an intermediate represented by Formula 4, 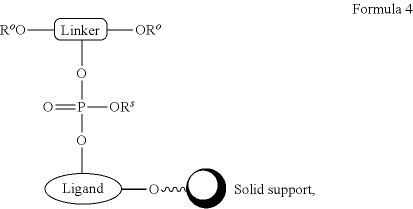
reacting the intermediate represented by Formula 4 with the RNA phosphoramidites to produce a compound represented by Formula 6, 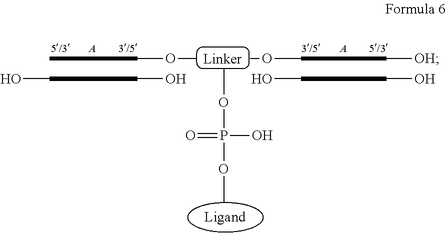
(b) reacting the compound of Formula 1 with the compound of Formula 3 to produce an intermediate represented by Formula 5, 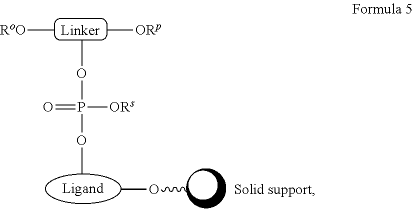
reacting the intermediate represented by Formula 5 with the RNA phosphoramidites to produce a compound represented by Formula 7, 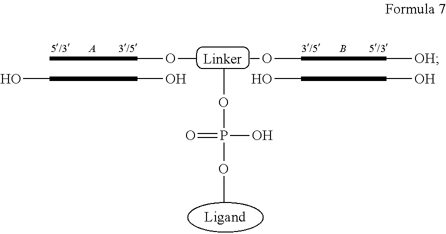
or
(c) reacting the compound represented by Formula 8 with the RNA phosphoramidites to produce an intermediate represented by Formula 10, 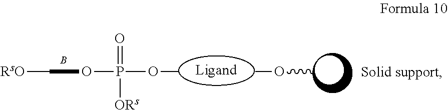
reacting the intermediate of Formula 10 with the compound of Formula 11 to produce an intermediate represented by Formula 12, 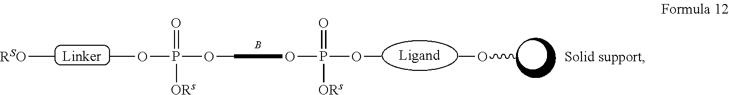
reacting the intermediate of Formula 12 with the RNA phosphoramidites to produce a compound represented by Formula 14, 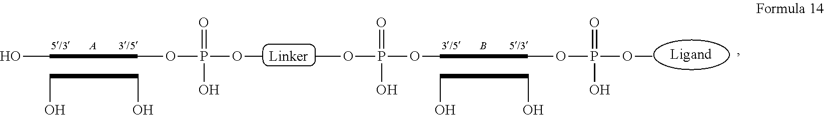
wherein A and B represent oligonucleotide fragments, R^{o} is a protecting group that is readily deprotected under acidic conditions, R^{p} is a protecting group that is readily deprotected under basic conditions, R^{q} is a C1-C3 alkyl group, R^{s} is a cyano C1-C3 alkyl group.

26. The method according to claim 25, wherein R^{o} is 4,4′-di-C1-C3 alkyl oxytrityl, R^{p} is 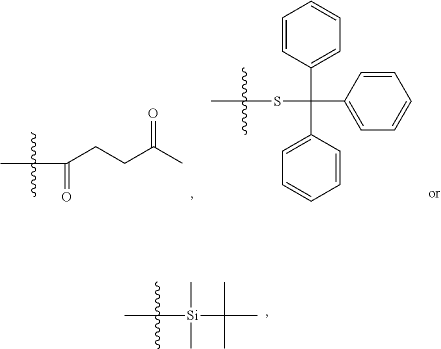
R^{q} is isopropyl, R^{s} is cyanoethyl.

27. The method according to claim 26, wherein R^{o} is

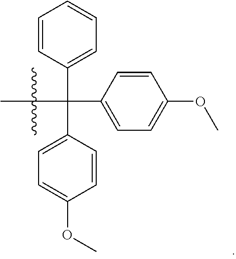 [SMILES: *C(c1ccccc1)(c1ccc(OC)cc1)c1ccc(OC)cc1]

28. A pharmaceutical composition comprising a compound of claim 17 and a pharmaceutically acceptable carrier.

29. A method of delivering two or more oligonucleotides to a subject in need thereof, the method comprising administering a compound of claim 17 or a pharmaceutical composition thereof.

30. A method for inhibiting expression of a target gene in a subject in need thereof, comprising administering to the subject a compound of claim 17 or a pharmaceutical composition thereof, wherein the compound or the pharmaceutical composition inhibits expression of the target gene.

31. A compound comprising the linker of claim 1 and two or more oligonucleotides covalently attached to the linker.


SEQUENCE LISTING (50 sequences)

SEQ ID NO: 1 - DNA, 21 bp, synthetic construct
  source 1..21: mol_type = other DNA
gtcatccacaatgagagtaca

SEQ ID NO: 2 - DNA, 21 bp, synthetic construct
  source 1..21: mol_type = other DNA
tctcccaccttttcttctaat

SEQ ID NO: 3 - DNA, 25 bp, synthetic construct
  source 1..25: mol_type = other DNA
tgtactgnctcattgtggatgacga

SEQ ID NO: 4 - DNA, 23 bp, synthetic construct
  source 1..23: mol_type = other DNA
attagaagaaaaggtgggagact

SEQ ID NO: 5 - DNA, 21 bp, synthetic construct
  source 1..21: mol_type = other DNA
ctagacctgttttgcttttgt

SEQ ID NO: 6 - DNA, 21 bp, synthetic construct
  source 1..21: mol_type = other DNA
ctagacctgttttgcttttgt

SEQ ID NO: 7 - DNA, 23 bp, synthetic construct
  source 1..23: mol_type = other DNA
acaaaagcaaaacaggtctagaa

SEQ ID NO: 8 - DNA, 21 bp, synthetic construct
  source 1..21: mol_type = other DNA
gcttgcaacaaagacatttat

SEQ ID NO: 9 - DNA, 21 bp, synthetic construct
  source 1..21: mol_type = other DNA
gcttgcaacaaagacatttat

SEQ ID NO: 10 - DNA, 23 bp, synthetic construct
  source 1..23: mol_type = other DNA
ataaatgtctttgttgcaagctt

SEQ ID NO: 11 - DNA, 23 bp, synthetic construct
  source 1..23: mol_type = other DNA
ataaatgtctttgttgcaagctt

SEQ ID NO: 12 - DNA, 21 bp, synthetic construct
  source 1..21: mol_type = other DNA
gagaaattctactacatctat

SEQ ID NO: 13 - DNA, 21 bp, synthetic construct
  source 1..21: mol_type = other DNA
aagcaagatatttttataata

SEQ ID NO: 14 - DNA, 23 bp, synthetic construct
  source 1..23: mol_type = other DNA
atagatgtagtagaatttctctg

SEQ ID NO: 15 - DNA, 23 bp, synthetic construct
  source 1..23: mol_type = other DNA
tattataaaaatatcttgctttt

SEQ ID NO: 16 - DNA, 21 bp, synthetic construct
  source 1..21: mol_type = other DNA
gtttgtgaaacaaaaaagtgt

SEQ ID NO: 17 - DNA, 21 bp, synthetic construct
  source 1..21: mol_type = other DNA
ctagacctgttttgcttttgt

SEQ ID NO: 18 - DNA, 23 bp, synthetic construct
  source 1..23: mol_type = other DNA
acacttttttgtttcacaaacaa

SEQ ID NO: 19 - DNA, 23 bp, synthetic construct
  source 1..23: mol_type = other DNA
acaaaagcaaaacaggtctagaa

SEQ ID NO: 20 - DNA, 21 bp, synthetic construct
  source 1..21: mol_type = other DNA
acatatttgatcagtcttttt

SEQ ID NO: 21 - DNA, 21 bp, synthetic construct
  source 1..21: mol_type = other DNA
ctagacctgttttgcttttgt

SEQ ID NO: 22 - DNA, 23 bp, synthetic construct
  source 1..23: mol_type = other DNA
aaaaagactgatcaaatatgttg

SEQ ID NO: 23 - DNA, 23 bp, synthetic construct
  source 1..23: mol_type = other DNA
acaaaagcaaaacaggtctagaa

SEQ ID NO: 24 - DNA, 21 bp, synthetic construct
  source 1..21: mol_type = other DNA
acatatttgatcagtcttttt

SEQ ID NO: 25 - DNA, 21 bp, synthetic construct
  source 1..21: mol_type = other DNA
ctagacctgttttgcttttgt

SEQ ID NO: 26 - DNA, 23 bp, synthetic construct
  source 1..23: mol_type = other DNA
aaaaagactgatcaaatatgttg

SEQ ID NO: 27 - DNA, 23 bp, synthetic construct
  source 1..23: mol_type = other DNA
acaaaagcaaaacaggtctagaa

SEQ ID NO: 28 - DNA, 23 bp, synthetic construct
  source 1..23: mol_type = other DNA
tgactttgaacacatgttgcttt

SEQ ID NO: 29 - DNA, 21 bp, synthetic construct
  source 1..21: mol_type = other DNA
ctagacctgttttgcttttgt

SEQ ID NO: 30 - DNA, 21 bp, synthetic construct
  source 1..21: mol_type = other DNA
agcaacatgtgttcaaagtca

SEQ ID NO: 31 - DNA, 23 bp, synthetic construct
  source 1..23: mol_type = other DNA
acaaaagcaaaacaggtctagaa

SEQ ID NO: 32 - DNA, 21 bp, synthetic construct
  source 1..21: mol_type = other DNA
tctcccaccttttcttctaat

SEQ ID NO: 33 - DNA, 21 bp, synthetic construct
  source 1..21: mol_type = other DNA
ctagacctgttttgcttttgt

SEQ ID NO: 34 - DNA, 23 bp, synthetic construct
  source 1..23: mol_type = other DNA
attagaagaaaaggtgggagact

SEQ ID NO: 35 - DNA, 21 bp, synthetic construct
  source 1..21: mol_type = other DNA
tctcccaccttttcttctaat

SEQ ID NO: 36 - DNA, 21 bp, synthetic construct
  source 1..21: mol_type = other DNA
ctagacctgttttgcttttgt

SEQ ID NO: 37 - DNA, 23 bp, synthetic construct
  source 1..23: mol_type = other DNA
attagaagaaaaggtgggagact

SEQ ID NO: 38 - DNA, 21 bp, synthetic construct
  source 1..21: mol_type = other DNA
ctagacctgttttgcttttgt

SEQ ID NO: 39 - DNA, 21 bp, synthetic construct
  source 1..21: mol_type = other DNA
ctagacctgttttgcttttgt

SEQ ID NO: 40 - DNA, 23 bp, synthetic construct
  source 1..23: mol_type = other DNA
acaaaagcaaaacaggtctagaa

SEQ ID NO: 41 - DNA, 21 bp, synthetic construct
  source 1..21: mol_type = other DNA
agcaacatgtgttcaaagtca

SEQ ID NO: 42 - DNA, 21 bp, synthetic construct
  source 1..21: mol_type = other DNA
ctagacctgttttgcttttgt

SEQ ID NO: 43 - DNA, 23 bp, synthetic construct
  source 1..23: mol_type = other DNA
tgactttgaacacatgttgcttt

SEQ ID NO: 44 - DNA, 21 bp, synthetic construct
  source 1..21: mol_type = other DNA
agcaacatgtgttcaaagtca

SEQ ID NO: 45 - DNA, 21 bp, synthetic construct
  source 1..21: mol_type = other DNA
ctagacctgttttgcttttgt

SEQ ID NO: 46 - DNA, 23 bp, synthetic construct
  source 1..23: mol_type = other DNA
tgactttgaacacatgttgcttt

SEQ ID NO: 47 - DNA, 21 bp, synthetic construct
  source 1..21: mol_type = other DNA
ctagacctgttttgcttttgt

SEQ ID NO: 48 - DNA, 21 bp, synthetic construct
  source 1..21: mol_type = other DNA
ctagacctgttttgcttttgt

SEQ ID NO: 49 - DNA, 21 bp, synthetic construct
  source 1..21: mol_type = other DNA
gagaaattctactacatctat

SEQ ID NO: 50 - DNA, 21 bp, synthetic construct
  source 1..21: mol_type = other DNA
gcaaacagcaatgcaaaacac